(19) United States
(12) Patent Application Publication
(10) Pub. No.: US 2009/0306100 A1
(43) Pub. Date: Dec. 10, 2009

(54) METHODS OF TREATING SCHIZOPHRENIA

(76) Inventors: Joseph Barbosa, Lambertville, NJ (US); Li Dong, Lawrenceville, NJ (US); Cynthia Anne Fink, Lebanon, NJ (US); Thomas Herbert Lanthom, The Woodlands, TX (US); Jian Cheng Wang, Addison, TX (US); G. Gregory Zipp, Robbinsville, NJ (US)

Correspondence Address: LEXICON PHARMACEUTICALS, INC., 8800 TECHNOLOGY FOREST PLACE, THE WOODLANDS, TX, 77381-1160, US

(21) Appl. No.: 12/484,337

(22) Filed: Jun. 15, 2009

Related U.S. Application Data
(62) Division of application No. 11/935,081, filed on Nov. 5, 2007, now abandoned.
(60) Provisional application No. 60/857,455, filed on Nov. 7, 2006.

(51) Int. Cl. A61K 31/519; A61K 31/497; A61K 31/506; A61P 25/18

(52) U.S. Cl. 514/265.1; 514/252.14; 514/275

(57) ABSTRACT

This invention relates to methods for treating, managing and preventing cognitive impairment associated with various diseases and disorders, age-associated memory impairment, and dementia.

This application is a divisional of U.S. application Ser. No. 11/935,081, filed Nov. 5, 2007, which claims priority to U.S. provisional application No. 60/857,455, filed Nov. 7, 2006, the entireties of which are incorporated herein by reference.

### 1. FIELD OF THE INVENTION

This invention relates to methods for treating, managing and preventing cognitive impairment associated with various diseases and disorders, age-associated memory impairment, and dementia.

### 2. BACKGROUND OF THE INVENTION

A large number of diseases and disorders are characterized by cognitive impairment. Some, like Fragile X syndrome, Down syndrome (trisomy 21), and Huntington's disease are genetic.

Fragile X syndrome is one of the most common forms of mental retardation. O'Donnell, W. T. and Warren, S. T, Annu. Rev. Neurosci 25:315-38 (2002). Patients suffering from the disease typically exhibit difficulties in thinking, problem solving, concept understanding, information processing and overall intelligence. See generally, “What is Fragile X” at http://www.fragilex.org/html/what.htm (2006).

As with those suffering from Fragile X syndrome, patients suffering from Down syndrome exhibit varying degrees of mental retardation. See generally, The Merck Manual, 17^{th} ed., 2233-36. Patients suffering from Huntington's disease exhibit progressively severe dementia or psychiatric disturbances, and ultimately lose the mental ability to care for themselves. Id. at 1465.

A need exits for methods of treating, managing and preventing cognitive impairment associated with these and other diseases and disorders.

### 3. SUMMARY OF THE INVENTION

This invention is directed, in part, to methods of improving cognitive performance in patients suffering from diseases and disorders, such as Attention-Deficit/Hyperactivity Disorder (ADD/ADHD), Down syndrome, Fragile X syndrome, Huntington's disease, Parkinson's disease, and schizophrenia. The invention also encompasses methods of treating, preventing and managing age-associated memory impairment and dementia.

Methods of the invention comprise decreasing proline transporter activity in a patient, either by administering an effective amount of a compound that inhibits the proline transporter or a compound that interferes with the expression of the gene that encodes the proline transporter.

### 4. BRIEF DESCRIPTION OF THE FIGURES

Certain aspects of the invention may be understood with reference to the attached figures.

FIG. 1 shows differences between wildtype and SLC6A7-knockout mice in a conditioned response test.

FIG. 2 shows the effect of a compound of the invention administered to mice prior to the learning phase of a conditioned response test.

FIG. 3 shows the effect of a compound of the invention administered to mice prior to a context test.

### 5. DETAILED DESCRIPTION OF THE INVENTION

This invention is based, in part, on the discovery that the proline transporter encoded by the human gene at map location 5q31-q32 (SLC6A7 gene; GENBANK accession no. NM_{—}014228) can be a potent modulator of mental performance in mammals. In particular, it has been found that genetically engineered mice that do not express a functional product of the murine ortholog of the SLC6A7 gene display significantly increased cognitive function, attention span, learning, and memory relative to control animals.

In view of this discovery, the protein product associated with the SLC6A7 coding region was used to discover compounds that may improve cognitive performance and may be useful in the treatment, prevention and/or management of diseases and disorders that affect cognitive performance.

### 5.1. Definitions

Unless otherwise indicated, the term “alkenyl” means a straight chain, branched and/or cyclic hydrocarbon having from 2 to 20 (e.g., 2 to 10 or 2 to 6) carbon atoms, and including at least one carbon-carbon double bond. Representative alkenyl moieties include vinyl, allyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, 1-hexenyl, 2-hexenyl, 3-hexenyl, 1-heptenyl, 2-heptenyl, 3-heptenyl, 1-octenyl, 2-octenyl, 3-octenyl, 1-nonenyl, 2-nonenyl, 3-nonenyl, 1-decenyl, 2-decenyl and 3-decenyl.

Unless otherwise indicated, the term “alkyl” means a straight chain, branched and/or cyclic (“cycloalkyl”) hydrocarbon having from 1 to 20 (e.g., 1 to 10 or 1 to 4) carbon atoms. Alkyl moieties having from 1 to 4 carbons are referred to as “lower alkyl.” Examples of alkyl groups include methyl, ethyl, propyl, isopropyl, n-butyl, t-butyl, isobutyl, pentyl, hexyl, isohexyl, heptyl, 4,4-dimethylpentyl, octyl, 2,2,4-trimethylpentyl, nonyl, decyl, undecyl and dodecyl. Cycloalkyl moieties may be monocyclic or multicyclic, and examples include cyclopropyl, cyclobutyl, cyclopentyl, cyclohexyl, and adamantyl. Additional examples of alkyl moieties have linear, branched and/or cyclic portions (e.g., 1-ethyl-4-methyl-cyclohexyl). The term “alkyl” includes saturated hydrocarbons as well as alkenyl and alkynyl moieties.

Unless otherwise indicated, the term “alkylaryl” or “alkyl-aryl” means an alkyl moiety bound to an aryl moiety.

Unless otherwise indicated, the term “alkylheteroaryl” or “alkyl-heteroaryl” means an alkyl moiety bound to a heteroaryl moiety.

Unless otherwise indicated, the term “alkylheterocycle” or “alkyl-heterocycle” means an alkyl moiety bound to a heterocycle moiety.

Unless otherwise indicated, the term “alkynyl” means a straight chain, branched or cyclic hydrocarbon having from 2 to 20 (e.g., 2 to 6) carbon atoms, and including at least one carbon-carbon triple bond. Representative alkynyl moieties include acetylenyl, propynyl, 1-butynyl, 2-butynyl, 1-pentynyl, 2-pentynyl, 3-methyl-1-butynyl, 4-pentynyl, 1-hexynyl, 2-hexynyl, 5-hexynyl, 1-heptynyl, 2-heptynyl, 6-heptynyl, 1-octynyl, 2-octynyl, 7-octynyl, 1-nonynyl, 2-nonynyl, 8-nonynyl, 1-decynyl, 2-decynyl and 9-decynyl.

Unless otherwise indicated, the term “alkoxy” means an —O-alkyl group. Examples of alkoxy groups include, but are not limited to, —OCH₃, —OCH₂CH₃, —O(CH₂)₂CH₃, —O(CH₂)₃CH₃, —O(CH₂)₄CH₃, and —O(CH₂)₅CH₃.

Unless otherwise indicated, the term “aryl” means an aromatic ring or an aromatic or partially aromatic ring system composed of carbon and hydrogen atoms. An aryl moiety may comprise multiple rings bound or fused together. Examples of aryl moieties include anthracenyl, azulenyl, biphenyl, fluorenyl, indan, indenyl, naphthyl, phenanthrenyl, phenyl, 1,2,3,4-tetrahydro-naphthalene, and tolyl.

Unless otherwise indicated, the term “arylalkyl” or “aryl-alkyl” means an aryl moiety bound to an alkyl moiety.

Unless otherwise indicated, the term “DTIC₅₀” means an IC₅₀ against human recombinant dopamine transporter as determined using the assay described in the Examples, below.

Unless otherwise indicated, the term “GTIC₅₀” means an IC₅₀ for human recombinant glycine transporter as determined using the assay described in the Examples, below.

Unless otherwise indicated, the terms “halogen” and “halo” encompass fluorine, chlorine, bromine, and iodine.

Unless otherwise indicated, the term “heteroalkyl” refers to an alkyl moiety (e.g., linear, branched or cyclic) in which at least one of its carbon atoms has been replaced with a heteroatom (e.g., N, O or S).

Unless otherwise indicated, the term “heteroaryl” means an aryl moiety wherein at least one of its carbon atoms has been replaced with a heteroatom (e.g., N, O or S). Examples include acridinyl, benzimidazolyl, benzofuranyl, benzoisothiazolyl, benzoisoxazolyl, benzoquinazolinyl, benzothiazolyl, benzoxazolyl, furyl, imidazolyl, indolyl, isothiazolyl, isoxazolyl, oxadiazolyl, oxazolyl, phthalazinyl, pyrazinyl, pyrazolyl, pyridazinyl, pyridyl, pyrimidinyl, pyrimidyl, pyrrolyl, quinazolinyl, quinolinyl, tetrazolyl, thiazolyl, and triazinyl.

Unless otherwise indicated, the term “heteroarylalkyl” or “heteroaryl-alkyl” means a heteroaryl moiety bound to an alkyl moiety.

Unless otherwise indicated, the term “heterocycle” refers to an aromatic, partially aromatic or non-aromatic monocyclic or polycyclic ring or ring system comprised of carbon, hydrogen and at least one heteroatom (e.g., N, O or S). A heterocycle may comprise multiple (i.e., two or more) rings fused or bound together. Heterocycles include heteroaryls. Examples include benzo[1,3]dioxolyl, 2,3-dihydro-benzo[1,4]dioxinyl, cinnolinyl, furanyl, hydantoinyl, morpholinyl, oxetanyl, oxiranyl, piperazinyl, piperidinyl, pyrrolidinonyl, pyrrolidinyl, tetrahydrofuranyl, tetrahydropyranyl, tetrahydropyridinyl, tetrahydropyrimidinyl, tetrahydrothiophenyl, tetrahydrothiopyranyl and valerolactamyl.

Unless otherwise indicated, the term “heterocyclealkyl” or “heterocycle-alkyl” refers to a heterocycle moiety bound to an alkyl moiety.

Unless otherwise indicated, the term “heterocycloalkyl” refers to a non-aromatic heterocycle.

Unless otherwise indicated, the term “heterocycloalkylalkyl” or “heterocycloalkyl-alkyl” refers to a heterocycloalkyl moiety bound to an alkyl moiety.

Unless otherwise indicated, the terms “manage,” “managing” and “management” encompass preventing the recurrence of the specified disease or disorder, or of one or more of its symptoms, in a patient who has already suffered from the disease or disorder, and/or lengthening the time that a patient who has suffered from the disease or disorder remains in remission. The terms encompass modulating the threshold, development and/or duration of the disease or disorder, or changing the way that a patient responds to the disease or disorder.

Unless otherwise indicated, the term “pharmaceutically acceptable salts” refers to salts prepared from pharmaceutically acceptable non-toxic acids or bases including inorganic acids and bases and organic acids and bases. Suitable pharmaceutically acceptable base addition salts include, but are not limited to, metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, phosphoric, sulfuric, and methanesulfonic acids. Examples of specific salts thus include hydrochloride and mesylate salts. Others are well-known in the art. See, e.g., Remington's Pharmaceutical Sciences (18th ed., Mack Publishing, Easton Pa.: 1990) and Remington: The Science and Practice of Pharmacy (19th ed., Mack Publishing, Easton Pa.: 1995).

Unless otherwise indicated, the term “potent proline transporter inhibitor” means a compound that has a PTIC₅₀ of less than about 200 nM.

Unless otherwise indicated, the terms “prevent,” “preventing” and “prevention” contemplate an action that occurs before a patient begins to suffer from the specified disease or disorder, which inhibits or reduces the severity of the disease or disorder, or of one or more of its symptoms. The terms encompass prophylaxis.

Unless otherwise indicated, a “prophylactically effective amount” of a compound is an amount sufficient to prevent a disease or condition, or one or more symptoms associated with the disease or condition, or to prevent its recurrence. A prophylactically effective amount of a compound means an amount of therapeutic agent, alone or in combination with other agents, which provides a prophylactic benefit in the prevention of the disease or condition. The term “prophylactically effective amount” can encompass an amount that improves overall prophylaxis or enhances the prophylactic efficacy of another prophylactic agent.

Unless otherwise indicated, the term “PTIC₅₀” means an IC₅₀ for human recombinant Na⁺-dependent proline transporter as determined using the assay described in the Examples, below.

Unless otherwise indicated, the term “substituted,” when used to describe a chemical structure or moiety, refers to a derivative of that structure or moiety wherein one or more of its hydrogen atoms is substituted with a chemical moiety or functional group such as, but not limited to, alcohol, aldehylde, alkoxy, alkanoyloxy, alkoxycarbonyl, alkenyl, alkyl (e.g., methyl, ethyl, propyl, t-butyl), alkynyl, alkylcarbonyloxy (—OC(O)alkyl), amide (—C(O)NH-alkyl- or -alkylNHC(O)alkyl), amidinyl (—C(NH)NH-alkyl or —C(NR)NH₂), amine (primary, secondary and tertiary such as alkylamino, arylamino, arylalkylamino), aroyl, aryl, aryloxy, azo, carbamoyl (—NHC(O)O-alkyl- or —OC(O)NH-alkyl), carbamyl (e.g., CONH₂, CONH-alkyl, CONH-aryl, and CONH-arylalkyl), carbonyl, carboxyl, carboxylic acid, carboxylic acid anhydride, carboxylic acid chloride, cyano, ester, epoxide, ether (e.g., methoxy, ethoxy), guanidino, halo, haloalkyl (e.g., —CCl₃, —CF₃, —C(CF₃)₃), heteroalkyl, hemiacetal, imine (primary and secondary), isocyanate, isothiocyanate, ketone, nitrile, nitro, oxo, phosphodiester, sulfide, sulfonamido (e.g., SO₂NH₂), sulfone, sulfonyl (including alkylsulfonyl, arylsulfonyl and arylalkylsulfonyl), sulfoxide, thiol (e.g., sulfhydryl, thioether) and urea (—NHCONH-alkyl-).

Unless otherwise indicated, a “therapeutically effective amount” of a compound is an amount sufficient to provide a therapeutic benefit in the treatment or management of a disease or condition, or to delay or minimize one or more symptoms associated with the disease or condition. A therapeutically effective amount of a compound means an amount of therapeutic agent, alone or in combination with other therapies, which provides a therapeutic benefit in the treatment or management of the disease or condition. The term “therapeutically effective amount” can encompass an amount that improves overall therapy, reduces or avoids symptoms or causes of a disease or condition, or enhances the therapeutic efficacy of another therapeutic agent.

Unless otherwise indicated, the terms “treat,” “treating” and “treatment” contemplate an action that occurs while a patient is suffering from the specified disease or disorder, which reduces the severity of the disease or disorder, or one or more of its symptoms, or retards or slows the progression of the disease or disorder.

Unless otherwise indicated, the term “include” has the same meaning as “include, but are not limited to,” and the term “includes” has the same meaning as “includes, but is not limited to.” Similarly, the term “such as” has the same meaning as the term “such as, but not limited to.”

Unless otherwise indicated, one or more adjectives immediately preceding a series of nouns is to be construed as applying to each of the nouns. For example, the phrase “optionally substituted alky, aryl, or heteroaryl” has the same meaning as “optionally substituted alky, optionally substituted aryl, or optionally substituted heteroaryl.”

It should be noted that a chemical moiety that forms part of a larger compound may be described herein using a name commonly accorded it when it exists as a single molecule or a name commonly accorded its radical. For example, the terms “pyridine” and “pyridyl” are accorded the same meaning when used to describe a moiety attached to other chemical moieties. Thus, the two phrases “XOH, wherein X is pyridyl” and “XOH, wherein X is pyridine” are accorded the same meaning, and encompass the compounds pyridin-2-ol, pyridin-3-ol and pyridin-4-ol.

It should also be noted that any atom shown in a drawing with unsatisfied valences is assumed to be attached to enough hydrogen atoms to satisfy the valences. In addition, chemical bonds depicted with one solid line parallel to one dashed line encompass both single and double (e.g., aromatic) bonds, if valences permit. Structures that represent compounds with one or more chiral centers, but which do not indicate stereochemistry (e.g., with bolded or dashed lines), encompasses pure stereoisomers and mixtures (e.g., racemic mixtures) thereof. Similarly, names of compounds having one or more chiral centers that do not specify the stereochemistry of those centers encompass pure stereoisomers and mixtures thereof.

### 5.2. Compounds

This invention encompasses compounds of formula I:

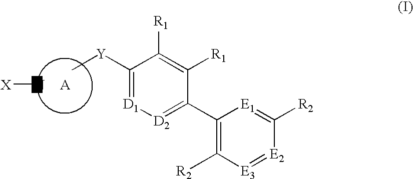

and pharmaceutically acceptable salts and solvates thereof, wherein: A is an optionally substituted non-aromatic heterocycle; each of D₁ and D₂ is independently N or CR₁; each of E₁, E₂ and E₃ is independently N or CR₂; X is optionally substituted heteroaryl; Y is O, C(O), CH(OH), or CH₂; each R₁ is independently hydrogen, halogen, cyano, R_{A}, OR_{A}, C(O)R_{A}, C(O)OR_{A}, C(O)N(R_{A}R_{B}), N(R_{A}R_{B}), or SO₂R_{A}; each R₂ is independently hydrogen, halogen, cyano, R_{A}, OR_{A}, C(O)R_{A}, C(O)OR_{A}, C(O)N(R_{A}R_{B}), N(R_{A}R_{B}), or SO₂R_{A}; each R_{A} is independently hydrogen or optionally substituted alkyl, aryl, arylalkyl, alkylaryl, heterocycle, heterocycle-alkyl, or alkyl-heterocycle; and each R_{B} is independently hydrogen or optionally substituted alkyl, aryl, arylalkyl, alkylaryl, heterocycle, heterocycle-alkyl, or alkyl-heterocycle.

One embodiment of the invention encompasses compounds of formula IA:

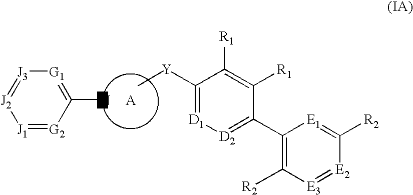

and pharmaceutically acceptable salts and solvates thereof.

Another encompasses compounds of formula IB:

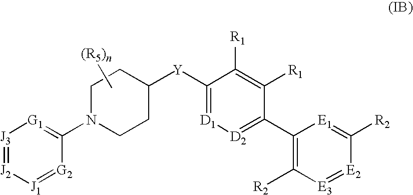

and pharmaceutically acceptable salts and solvates thereof, wherein: each R₅ is independently halogen, cyano, R_{5A}, OR_{5A}, C(O)R_{5A}, C(O)OR_{5A}, C(O)N(R_{5A}R_{5B}), N(R_{5A}R_{5B}), or SO₂R_{5A}; each R_{5A} is independently hydrogen or optionally substituted alkyl, aryl, arylalkyl, alkylaryl, heterocycle, heterocycle-alkyl, or alkyl-heterocycle; each R_{5B} is independently hydrogen or optionally substituted alkyl, aryl, arylalkyl, alkylaryl, heterocycle, heterocycle-alkyl, or alkyl-heterocycle; and n is 0-5.

Another encompasses compounds of formula IC:

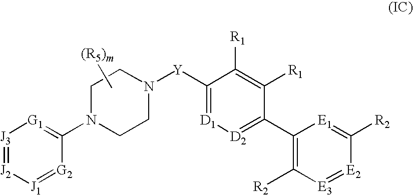

and pharmaceutically acceptable salts and solvates thereof, wherein: Y is O, C(O) or CH₂; each R₅ is independently halogen, cyano, R_{5A}, OR_{5A}, C(O)R_{5A}, C(O)OR_{5A}, C(O)N(R_{5A}R_{5B}), N(R_{5A}R_{5B}), or SO₂R_{5A}; each R_{5A} is independently hydrogen or optionally substituted alkyl, aryl, arylalkyl, alkylaryl, heterocycle, heterocycle-alkyl, or alkyl-heterocycle; each R_{5B} is independently hydrogen or optionally substituted alkyl, aryl, arylalkyl, alkylaryl, heterocycle, heterocycle-alkyl, or alkyl-heterocycle; and m is 0-4.

Another encompasses compounds of formula ID:

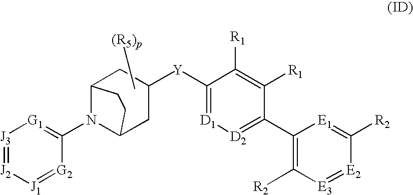

and pharmaceutically acceptable salts and solvates thereof, wherein: each R₅ is independently halogen, cyano, R_{5A}, OR_{5A}, C(O)R_{5A}, C(O)OR_{5A}, C(O)N(R_{5A}R_{5B}), N(R_{5A}R_{5B}), or SO₂R_{5A}; each R_{5A} is independently hydrogen or optionally substituted alkyl, aryl, arylalkyl, alkylaryl, heterocycle, heterocycle-alkyl, or alkyl-heterocycle; each R_{5B} is independently hydrogen or optionally substituted alkyl, aryl, arylalkyl, alkylaryl, heterocycle, heterocycle-alkyl, or alkyl-heterocycle; and p is 0-7.

Another encompasses compounds of formula IE:

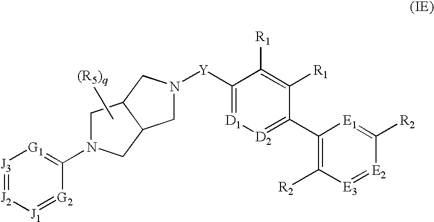

and pharmaceutically acceptable salts and solvates thereof, wherein: Y is O, C(O) or CH₂; each R₅ is independently halogen, cyano, R_{5A}, OR_{5A}, C(O)R_{5A}, C(O)OR_{5A}, C(O)N(R_{5A}R_{5B}), N(R_{5A}R_{5B}), or SO₂R_{5A}; each R_{5A} is independently hydrogen or optionally substituted alkyl, aryl, arylalkyl, alkylaryl, heterocycle, heterocycle-alkyl, or alkyl-heterocycle; each R_{5B} is independently hydrogen or optionally substituted alkyl, aryl, arylalkyl, alkylaryl, heterocycle, heterocycle-alkyl, or alkyl-heterocycle; and q is 0-6.

Another encompasses compounds of formula IF:

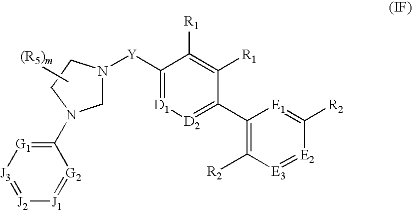

and pharmaceutically acceptable salts and solvates thereof, wherein: each R₅ is independently halogen, cyano, R_{5A}, OR_{5A}, C(O)R_{5A}, C(O)OR_{5A}, C(O)N(R_{5A}R_{5B}), N(R_{5A}R_{5B}), or SO₂R_{5A}; each R_{5A} is independently hydrogen or optionally substituted alkyl, aryl, arylalkyl, alkylaryl, heterocycle, heterocycle-alkyl, or alkyl-heterocycle; each R_{5B} is independently hydrogen or optionally substituted alkyl, aryl, arylalkyl, alkylaryl, heterocycle, heterocycle-alkyl, or alkyl-heterocycle; and m is 0-4.

Another encompasses compounds of formula II:

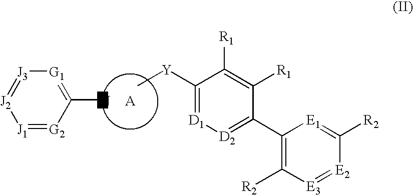

and pharmaceutically acceptable salts and solvates thereof, wherein: A is an optionally substituted non-aromatic heterocycle; each of D₁ and D₂ is independently N or CR₁; each of E₁, E₂ and E₃ is independently N or CR₂; each of G₁ and G₂ are independently N or CR₃; each of J₁, J₂ and J₃ are independently N or CR₄; Y is O, C(O), CH(OH), or CH₂; each R₁ is independently hydrogen, halogen, or (C₁₋₁₀)alkyl; each R₂ is independently halogen, cyano, R_{2A}, OR_{2A}, or SO₂R_{2A}; each R_{2A} is independently hydrogen or (C₁₋₁₀)alkyl optionally substituted with one or more halogens; each R₃ is independently hydrogen, cyano, or (C₁₋₁₀)alkyl optionally substituted with one or more halogens; and each R₄ is independently hydrogen, cyano, or (C₁₋₁₀)alkyl optionally substituted with one or more halogens.

Another encompasses compounds of formula IIA:

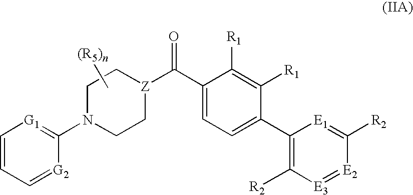

and pharmaceutically acceptable salts and solvates thereof, wherein: Z is CR₅ or N; each R₅ is independently halogen, cyano, R_{5A}, OR_{5A}, C(O)R_{5A}, C(O)OR_{5A}, C(O)N(R_{5A}R_{5B}), N(R_{5A}R_{5B}), or SO₂R_{5A}; each R_{5A} is independently hydrogen or optionally substituted alkyl, aryl, arylalkyl, alkylaryl, heterocycle, heterocycle-alkyl, or alkyl-heterocycle; each R_{5B} is independently hydrogen or optionally substituted alkyl, aryl, arylalkyl, alkylaryl, heterocycle, heterocycle-alkyl, or alkyl-heterocycle; and n is 0-5 if Z is CR₅, or 0-4 if Z is N.

Another encompasses compounds of formula IIB:

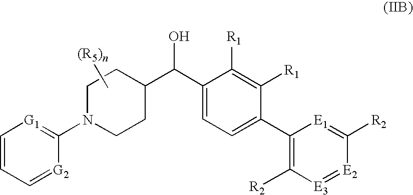

and pharmaceutically acceptable salts and solvates thereof.

Another encompasses compounds of formula IIC:

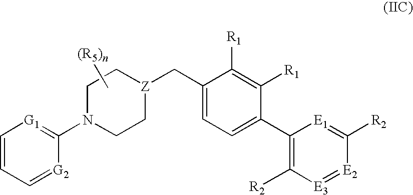

and pharmaceutically acceptable salts and solvates thereof, wherein: Z is CR₅ or N; each R₅ is independently halogen, cyano, R_{5A}, OR_{5A}, C(O)R_{5A}, C(O)OR_{5A}, C(O)N(R_{5A}R_{5B}), N(R_{5A}R_{5B}), or SO₂R_{5A}; each R_{5A} is independently hydrogen or optionally substituted alkyl, aryl, arylalkyl, alkylaryl, heterocycle, heterocycle-alkyl, or alkyl-heterocycle; each R_{5B} is independently hydrogen or optionally substituted alkyl, aryl, arylalkyl, alkylaryl, heterocycle, heterocycle-alkyl, or alkyl-heterocycle; and n is 0-5 if Z is CR₅, or 0-4 if Z is N.

In one embodiment of the invention encompassed by formula II (and IIA-C, as appropriate), at least one of G₁, G₂, J₁, J₂ or J₃ is N. In another, at least one of J₁, J₂ and J₃ is CR₄, In another, if Y is C(O), A is piperazine, all of G₁, G₂, J₁, J₃, D₁, D₂, E₁, and E₃ are CH, and all of R₁ are hydrogen, then none of R₂ are lower alkyl. In another, if Y is C(O), A is piperazine, D₂ and E₁ are both N, and all of R₁ and R₂ are hydrogen, then R₄ is not cyano. In another, if Y is O, A is pyrrolidine, all of G₁, G₂, J₁, J₃, D₁, D₂, E₁, E₂, and E₃ are CH, and all of R₁ are hydrogen, then at least one R₂ is not hydrogen. In another, if Y is CH₂, A is piperazine, all of G₂, J₁, J₂, J₃, D₁, and D₂ are CH, all of E₁, E₂ and E₃ are CR₂, and all of R₁ are hydrogen, at least one R₂ is not hydrogen. In another, if Y is C(O) or CH₂, A is piperazine, at least one of G₁ and G₂ is N, all of J₁, J₂, J₃, D₁, D₂, E₁, E₂ and E₃ are CH, and all of R₁ are hydrogen, then at least one R₂ is not hydrogen.

Various other embodiments of the invention, which pertain to each of the above formulae (e.g., I, IA-F, II and IIA-C) when appropriate (when the particular formula contains the moiety referred to), are as follows.

In one, A is optionally substituted non-aromatic heterocycle containing no more than two nitrogen atoms (i.e., the heterocycle, which contains no more than two nitrogen atoms, is optionally substituted).

In another, A is monocyclic. In another, A is bicyclic. In another, A is unsubstituted. In another, A is optionally substituted pyrrolidine, piperidine, piperazine, hexahydropyrimidine, 1,2,3,6-tetrahydropyridine, octahydrocyclopenta[c]pyrrole, or octahydropyrrolo[3,4-c]pyrrole.

In another, one of D₁ and D₂ is N. In another, both D₁ and D₂ are N. In another, both D₁ and D₂ are CR₁.

In another, one of E₁, E₂ and E₃ is N. In another, two of E₁, E₂ and E₃ are N. In another, all of E₁, E₂ and E₃ are N. In another, all of E₁, E₂ and E₃ are independently CR₂.

In another, R₁ is hydrogen, halogen, or optionally substituted alkyl. In another, R₁ is OR_{A} and, for example, R_{A} is hydrogen or optionally substituted alkyl.

In another, R₂ is hydrogen, halogen, or optionally substituted alkyl. In another, R₂ is OR_{A} and, for example, R_{A} is hydrogen or optionally substituted alkyl.

In another, X is an optionally substituted 5-, 6-, 9- or 10-membered heteroaryl. In another, X is optionally substituted 5- or 6-membered heteroaryl. In another, X is of the formula:

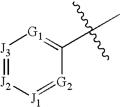

wherein: each of G₁ and G₂ are independently N or CR₃; each of J₁, J₂ and J₃ are independently N or CR₄; each R₃ is independently hydrogen, halogen, cyano, R_{A}, OR_{A}, C(O)R_{A}, C(O)OR_{A}, C(O)N(R_{A}R_{B}), N(R_{A}R_{B}), or SO₂R_{A}; and each R₄ is independently hydrogen, halogen, cyano, R_{A}, OR_{A}, C(O)R_{A}, C(O)OR_{A}, C(O)N(R_{A}R_{B}), N(R_{A}R_{B}), or SO₂R_{A}; provided that at least one of J₁, J₂ and J₃ is CR₄.

In another, one of G₁ and G₂ is N. In another, both G₁ and G₂ are N. In another, both G₁ and G₂ are CR₃.

In another, one of J₁, J₂ and J₃ is N. In another, two of J₁, J₂ and J₃ are N. In another, all of J₁, J₂ and J₃ are independently CR₄.

In another, R₃ is hydrogen, halogen, or optionally substituted alkyl. In another, R₃ is OR_{A} and, for example, R_{A} is hydrogen or optionally substituted alkyl.

In another, R₄ is hydrogen, halogen, or optionally substituted alkyl. In another, R₄ is OR_{A} and, for example, R_{A} is hydrogen or optionally substituted alkyl.

In another, Y is C(O). In another, Y is CH(OH). In another, Y is CH₂.

This invention also encompasses compounds of formula III:

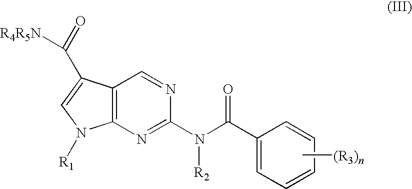

and pharmaceutically acceptable salts and solvates thereof, wherein: R₁ is hydrogen or optionally substituted alkyl, aryl, heterocycle, alkyl-aryl or alkyl-heterocycle; R₂ is hydrogen or optionally substituted alkyl; each R₃ is independently halogen, amine, hydroxy, alkoxy, or optionally substituted alkyl, aryl or heterocycle; R₄ and R₅ are each independently hydrogen or optionally substituted alkyl, aryl, heterocycle, alkyl-aryl or alkyl-heterocycle, or taken together with the nitrogen atom to which they are attached, form an optionally substituted heterocycle; and n is 0 to 5.

In one embodiment, R₁ is t-butyl or propyl. In another embodiment, R₃ is lower alkyl. In another embodiment, R₄ and R₅ are taken together to form optionally substituted pyridine or pyrrolidine. In another embodiment, R₄ and R₅ together with the nitrogen atom to which they are attached do not form 1,4-diaza-bicyclo[3.2.2]nonane. In another embodiment, R₄ and R₅ together with the nitrogen atom to which they are attached do not form piperazine-C(O)-aryl (e.g., piperazine-C(O)-phenyl).

This invention also encompasses compounds of formula IIIA:

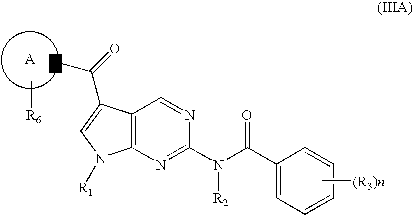

and pharmaceutically acceptable salts and solvates thereof, wherein: A is a heterocycle; R₁ is hydrogen or optionally substituted alkyl, aryl, heterocycle, alkyl-aryl or alkyl-heterocycle; R₂ is hydrogen or optionally substituted alkyl; each R₃ is independently halogen, amine, hydroxy, alkoxy, or optionally substituted alkyl, aryl or heterocycle; R₆ is optionally substituted alkyl, aryl, heterocycle, alkyl-aryl or alkyl-heterocycle; and n is 0 to 5.

In one embodiment, A is optionally substituted pyridine or pyrrolidine. In another embodiment, R₆ is pyridine or pyrrolidine. In another embodiment, R₄ and R₅ together with the nitrogen atom to which they are attached do not form 1,4-diaza-bicyclo[3.2.2]-nonane. In another embodiment, R₄ and R₅ together with the nitrogen atom to which they are attached do not form piperazine-C(O)-aryl (e.g., piperazine-C(O)-phenyl).

This invention also encompasses compounds of formula IV:

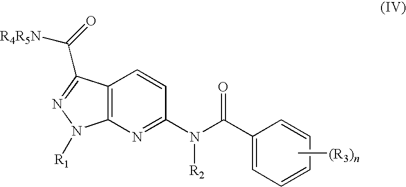

and pharmaceutically acceptable salts and solvates thereof, wherein: R₁ is hydrogen or optionally substituted alkyl, aryl, heterocycle, alkyl-aryl or alkyl-heterocycle; R₂ is hydrogen or optionally substituted alkyl; each R₃ is independently halogen, amine, hydroxy, alkoxy, or optionally substituted alkyl, aryl or heterocycle; R₄ and R₅ are each independently hydrogen, or optionally substituted alkyl, aryl, heterocycle, alkyl-aryl or alkyl-heterocycle, or taken together with the nitrogen atom to which they are attached, form an optionally substituted heterocycle; and n is 0 to 5.

In one embodiment, R₁ is t-butyl or propyl. In another embodiment, R₃ is lower alkyl. In another embodiment, R₄ and R₅ are taken together to form optionally substituted pyridine or pyrrolidine. In another embodiment, R₄ and R₅ together with the nitrogen atom to which they are attached do not form 1,4-diaza-bicyclo[3.2.2]nonane. In another embodiment, R₄ and R₅ together with the nitrogen atom to which they are attached do not form piperazine-C(O)-aryl (e.g., piperazine-C(O)-phenyl).

This invention also encompasses compounds of formula IVA:

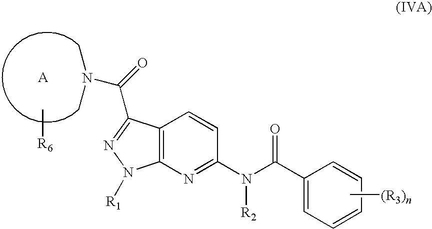

and pharmaceutically acceptable salts and solvates thereof, wherein: A is a heterocycle; R₁ is hydrogen or optionally substituted alkyl, aryl, heterocycle, alkyl-aryl or alkyl-heterocycle; R₂ is hydrogen or optionally substituted alkyl; each R₃ is independently halogen, amine, hydroxy, alkoxy, or optionally substituted alkyl, aryl or heterocycle; R₆ is optionally substituted alkyl, aryl, heterocycle, alkyl-aryl or alkyl-heterocycle; and n is 0 to 5.

In one embodiment, A is optionally substituted pyridine or pyrrolidine. In another embodiment, R₆ is pyridine or pyrrolidine. In another embodiment, R₄ and R₅ together with the nitrogen atom to which they are attached do not form 1,4-diaza-bicyclo[3.2.2]-nonane. In another embodiment, R₄ and R₅ together with the nitrogen atom to which they are attached do not form piperazine-C(O)-aryl (e.g., piperazine-C(O)-phenyl).

This invention also encompasses compounds of formula V:

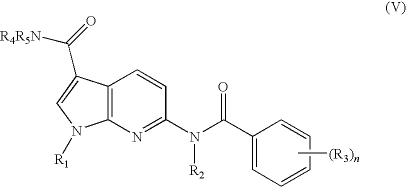

and pharmaceutically acceptable salts and solvates thereof, wherein: R₁ is hydrogen or optionally substituted alkyl, aryl, heterocycle, alkyl-aryl or alkyl-heterocycle; R₂ is hydrogen or optionally substituted alkyl; each R₃ is independently halogen, amine, hydroxy, alkoxy, or optionally substituted alkyl, aryl or heterocycle; R₄ and R₅ are each independently hydrogen, or optionally substituted alkyl, aryl, heterocycle, alkyl-aryl or alkyl-heterocycle, or taken together with the nitrogen atom to which they are attached, form an optionally substituted heterocycle; and n is 0 to 5.

In one embodiment, R₁ is t-butyl or propyl. In another embodiment, R₃ is lower alkyl. In another embodiment, R₄ and R₅ are taken together to form optionally substituted pyridine or pyrrolidine. In another embodiment, R₄ and R₅ together with the nitrogen atom to which they are attached do not form 1,4-diaza-bicyclo[3.2.2]nonane. In another embodiment, R₄ and R₅ together with the nitrogen atom to which they are attached do not form piperazine-C(O)-aryl (e.g., piperazine-C(O)-phenyl).

This invention also encompasses compounds of formula VA:

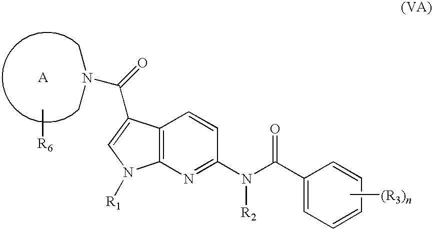

and pharmaceutically acceptable salts and solvates thereof, wherein: A is a heterocycle; R₁ is hydrogen or optionally substituted alkyl, aryl, heterocycle, alkyl-aryl or alkyl-heterocycle; R₂ is hydrogen or optionally substituted alkyl; each R₃ is independently halogen, amine, hydroxy, alkoxy, or optionally substituted alkyl, aryl or heterocycle; R₆ is optionally substituted alkyl, aryl, heterocycle, alkyl-aryl or alkyl-heterocycle; and n is 0 to 5.

In one embodiment, A is optionally substituted pyridine or pyrrolidine. In another embodiment, R₆ is pyridine or pyrrolidine. In another embodiment, A is not 1,4-diaza-bicyclo[3.2.2]nonane. In another embodiment, A is not piperazine-C(O)-aryl (e.g., piperazine-C(O)-phenyl).

Examples of specific compounds include:
(1-(pyrimidin-2-yl)piperidin-4-yl)(4′-(trifluoromethyl)biphenyl-4-yl)methanol;
(4′-chlorobiphenyl-4-yl)(2,6-dimethyl-4-(pyridin-2-yl)piperazin-1-yl)methanone;
(3′-chloro-3-methoxybiphenyl-4-yl)(1-(pyrimidin-2-yl)piperidin-4-yl)methanone;
(4-(pyrimidin-2-yl)piperazin-1-yl)(4′-(trifluoromethyl)biphenyl-4-yl)methanone;
(3-fluoro-4′-methylbiphenyl-4-yl)(4-(pyrimidin-2-yl)piperazin-1-yl)methanone;
(4′-chlorobiphenyl-4-yl)(4-(pyrimidin-2-yl)piperazin-1-yl)methanone;
(2′-methylbiphenyl-4-yl)(4-(pyrimidin-2-yl)piperazin-1-yl)methanone;
(4-(benzo[d]oxazol-2-yl)piperazin-1-yl)(4′-(trifluoromethyl)biphenyl-4-yl)methanone;
biphenyl-4-yl(4-(4-(trifluoromethyl)pyrimidin-2-yl)piperazin-1-yl)methanone;
(S)-(2-benzyl-4-(pyrimidin-2-yl)piperazin-1-yl)(4′-(trifluoromethyl)biphenyl-4-yl)methanone;
(5-(pyrimidin-2-yl)hexahydropyrrolo[3,4-c]pyrrol-2(1H)-yl)(6-p-tolylpyridin-3-yl)methanone;
(5-(pyrimidin-2-yl)hexahydropyrrolo[3,4-c]pyrrol-2(1H)-yl)(6-(3-(trifluoromethyl)phenyl)pyridin-3-yl)methanone;
(6-(4-chlorophenyl)pyridin-3-yl)(5-(pyrimidin-2-yl)hexahydropyrrolo[3,4-c]pyrrol-2(1H)-yl)methanone;
(5-(pyrimidin-2-yl)hexahydropyrrolo[3,4-c]pyrrol-2(1H)-yl)(6-(4-(trifluoromethyl)phenyl)pyridin-3-yl)methanone;
(5-(4-chlorophenyl)isoxazol-3-yl)(4-(pyrimidin-2-yl)piperazin-1-yl)methanone;
(3′-chlorobiphenyl-4-yl)(1-(pyridin-2-yl)piperidin-4-yl)methanone;
biphenyl-4-yl(4-(pyrimidin-2-yl)-1,4-diazepan-1-yl)methanone;
(8-(pyrimidin-2-yl)-8-azabicyclo[3.2.1]octan-3-yl)(4′-(trifluoromethyl)biphenyl-4-yl)methanone;
biphenyl-4-yl(1-(pyrimidin-2-yl)azetidin-3-yl)methanone;
(6-(4-chloro-3-(trifluoromethyl)phenyl)pyridin-3-yl)(4-(pyrimidin-2-yl)piperazin-1-yl)methanone;
(6-(4-chloro-3-methylphenyl)pyridin-3-yl)(4-(pyrimidin-2-yl)piperazin-1-yl)methanone;
(4′-chlorobiphenyl-4-yl)(1-(pyridin-2-yl)piperidin-4-yl)methanone;
(2-methylbiphenyl-4-yl)(4-(pyrimidin-2-yl)piperazin-1-yl)methanone;
(3,4′-dimethylbiphenyl-4-yl)(4-(pyrimidin-2-yl)piperazin-1-yl)methanone;
(5-(3-chlorophenyl)pyridin-2-yl)(4-(pyrimidin-2-yl)piperazin-1-yl)methanone;
(4-(pyrimidin-2-yl)piperazin-1-yl)(5-p-tolylpyridin-2-yl)methanone;
(4-(pyridin-2-yl)piperazin-1-yl)(3′-(trifluoromethyl)biphenyl-4-yl)methanone;
(1-(pyrimidin-2-yl)piperazin-4-yl)(4′-(trifluoromethyl)biphenyl-4-yl)methanone;
(3-fluoro-3′-(trifluoromethyl)biphenyl-4-yl)(4-(pyrimidin-2-yl)piperazin-1-yl)methanone;
(4-(pyrimidin-2-yl)piperazin-1-yl)(3′-(trifluoromethoxy)biphenyl-4-yl)methanone;
(5-(pyrimidin-2-yl)hexahydropyrrolo[3,4-c]pyrrol-2(1H)-yl)(3′-(trifluoromethyl)biphenyl-4-yl)methanone;
biphenyl-4-yl(5-(pyrimidin-2-yl)hexahydropyrrolo[3,4-c]pyrrol-2(1H)-yl)methanone;
(1-phenyl-5-(pyrimidin-2-yl)hexahydropyrrolo[3,4-c]pyrrol-2(1H)-yl)(4′-(trifluoromethyl)biphenyl-4-yl)methanone;
biphenyl-4-yl(4-(thiazol-2-yl)piperazin-1-yl)methanone;
(4-(4-chlorophenyl)cyclohexyl)(4-(pyrimidin-2-yl)piperazin-1-yl)methanone;
4′-(4-(pyrimidin-2-yl)piperazine-1-carbonyl)biphenyl-3-carbonitrile;
(4′-(methylsulfonyl)biphenyl-4-yl)(4-(pyrimidin-2-yl)piperazin-1-yl)methanone;
2-(4-((3′-chlorobiphenyl-4-yl)(hydroxy)methyl)piperidin-1-yl)pyrimidin-5-ol;
(4-(pyridin-3-yl)phenyl)(4-(pyrimidin-2-yl)piperazin-1-yl)methanone;
(3′-chloro-3-hydroxybiphenyl-4-yl)(1-(pyrimidin-2-yl)piperidin-4-yl)methanone;
1-(4′-(4-(pyrimidin-2-yl)piperazine-1-carbonyl)biphenyl-3-yl)ethanone;
(2′,4′-difluoro-3-methylbiphenyl-4-yl)(4-(pyrimidin-2-yl)piperazin-1-yl)methanone;
(5-phenyl-1H-pyrrol-2-yl)(4-(pyrimidin-2-yl)piperazin-1-yl)methanone;
(6-(4-chlorophenyl)pyridin-3-yl)(4-(pyrimidin-2-yl)piperazin-1-yl)methanone;
(5′-chloro-2′-fluorobiphenyl-4-yl)(8-(pyrimidin-2-yl)-8-azabicyclo[3.2.1]octan-3-yl)methanone;
2-(4-(biphenylcarbonyl)piperazin-1-yl)nicotinonitrile;
2-(4-(biphenyl-4-yloxy)piperidin-1-yl)pyrimidine;
(2′-fluoro-5′-(trifluoromethyl)biphenyl-4-yl)(1-(pyrimidin-2-yl)pyrrolidin-3-yl)methanone;
(4-(4-methylthiophen-2-yl)phenyl)(1-(pyrimidin-2-yl)piperidin-4-yl)methanone;
(4′-fluorobiphenyl-4-yl)(4-(pyrimidin-2-yl)piperazin-1-yl)methanone;
(2-fluoro-4′-methylbiphenyl-4-yl)(4-(pyrimidin-2-yl)piperazin-1-yl)methanone;
biphenyl-4-yl(3-methyl-4-(pyrimidin-2-yl)piperazin-1-yl)methanone;
(2′-fluoro-5′-(trifluoromethyl)biphenyl-4-yl)(4-methyl-1-(pyrimidin-2-yl)piperidin-4-yl)methanone;
biphenyl-4-yl(4-(5-methylpyridin-2-yl)piperazin-1-yl)methanone;
biphenyl-4-yl(2-methyl-4-(pyrimidin-2-yl)piperazin-1-yl)methanone;
(1-(pyridin-2-yl)piperidin-4-yl)(3′-(trifluoromethyl)biphenyl-4-yl)methanone;
(6-(3-chlorophenyl)pyridin-3-yl)(4-(pyrimidin-2-yl)piperazin-1-yl)methanone;
(4-(pyrimidin-2-yl)piperazin-1-yl)(6-(3-(trifluoromethyl)phenyl)pyridin-3-yl)methanone;
(4-(pyrimidin-2-yl)piperazin-1-yl)(6-p-tolylpyridin-3-yl)methanone;
(4′-chloro-3′-(trifluoromethyl)biphenyl-4-yl)(1-(pyrimidin-2-yl)piperidin-4-yl)methanone;
(4-(2-chloropyridin-4-yl)phenyl)(4-(pyrimidin-2-yl)piperazin-1-yl)methanone;
(2′,4′-difluorobiphenyl-4-yl)(4-(pyrimidin-2-yl)piperazin-1-yl)methanone;
(6-(2,4-difluorophenyl)pyridin-3-yl)(4-(pyrimidin-2-yl)piperazin-1-yl)methanone;
(3′,5′-dichlorobiphenyl-4-yl)(4-(pyrimidin-2-yl)piperazin-1-yl)methanone;
2-(4-(biphenyl-4-ylmethyl)piperazin-1-yl)pyrimidine;
(4′-chlorobiphenyl-4-yl)(1-(pyrimidin-2-yl)piperidin-4-yl)methanone;
(6-(3-chlorophenyl)pyridin-3-yl)(5-(pyrimidin-2-yl)hexahydropyrrolo[3,4-c]pyrrol-2(1H)-yl)methanone;
(1-(pyridin-2-yl)piperidin-4-yl)(4′-(trifluoromethyl)biphenyl-4-yl)methanone;
(2′-fluoro-5′-(trifluoromethyl)biphenyl-4-yl)(4-(pyrimidin-2-yl)piperazin-1-yl)methanone;
(4′-methylbiphenyl-4-yl)(4-(pyrimidin-2-yl)piperazin-1-yl)methanone;
biphenyl-4-yl(4-(5-methylpyridin-2-yl)piperazin-1-yl)methanone;
1-(biphenylcarbonyl)-4-(pyrimidin-2-yl)piperazin-2-one;
biphenyl-4-yl(1-(pyrimidin-2-yl)-1,2,3,6-tetrahydropyridin-4-yl)methanone;
(3′-chlorobiphenyl-4-yl)(1-(pyrimidin-2-yl)piperidin-4-yl)methanone;
biphenyl-4-yl(1-(pyrimidin-2-yl)-1,2,3,6-tetrahydropyridin-4-yl)methanol;
(3′-chlorobiphenyl-4-yl)(4-(pyrimidin-2-yl)piperazin-1-yl)methanone;
(4-(pyrimidin-2-yl)piperazin-1-yl)(3′-(trifluoromethyl)biphenyl-4-yl)methanone;
(3′-chlorobiphenyl-4-yl)(1-(5-hydroxypyrimidin-2-yl)piperidin-4-yl)methanone;
(4′-ethylbiphenyl-4-yl)(4-(pyrimidin-2-yl)piperazin-1-yl)methanone;
biphenyl-4-yl(4-(4-methylpyrimidin-2-yl)piperazin-1-yl)methanone;
(6-(2,4-difluorophenyl)pyridin-3-yl)(5-(pyrimidin-2-yl)hexahydropyrrolo[3,4-c]pyrrol-2(1H)-yl)methanone;
(4′-chlorobiphenyl-4-yl)(4-(pyridin-2-yl)piperazin-1-yl)methanone;
(5-methyl-1-(pyrimidin-2-yl)-1,2,3,6-tetrahydropyridin-4-yl)(4′-(trifluoromethyl)biphenyl-4-yl)methanone;
biphenyl-4-yl(4-(5-ethylpyrimidin-2-yl)piperazin-1-yl)methanone;
(4-(pyridin-2-yl)piperazin-1-yl)(4′-(trifluoromethyl)biphenyl-4-yl)methanone;
(4-(pyridin-2-yl)phenyl)(4-(pyrimidin-2-yl)piperazin-1-yl)methanone;
biphenyl-4-yl(4-(pyrazin-2-yl)piperazin-1-yl)methanone;
(4′-methoxybiphenyl-4-yl)(4-(pyrimidin-2-yl)piperazin-1-yl)methanone;
biphenyl-4-yl(4-(6-methylpyridazin-3-yl)piperazin-1-yl)methanone;
4′-(4-(pyrimidin-2-yl)piperazine-1-carbonyl)biphenyl-4-carbonitrile;
(2,6-dimethyl-4-(pyridin-2-yl)piperazin-1-yl)(4′-(trifluoromethyl)biphenyl-4-yl)methanone;
(5-phenylthiophen-2-yl)(4-(pyrimidin-2-yl)piperazin-1-yl)methanone;
(6-(5-methylthiophen-2-yl)pyridin-3-yl)(4-(pyrimidin-2-yl)piperazin-1-yl)methanone;
biphenyl-4-yl(4-(pyridin-4-yl)piperazin-1-yl)methanone;
(R)-(2-methyl-4-(pyrimidin-2-yl)piperazin-1-yl)(4′-(trifluoromethyl)biphenyl-4-yl)methanone;
biphenyl-4-yl((2S,5S)-2,5-dimethyl-4-(pyrimidin-2-yl)piperazin-1-yl)methanone;
(3′-chlorobiphenyl-4-yl)(4-(pyridin-2-yl)piperazin-1-yl)methanone;
(4-(pyrimidin-2-yl)piperazin-1-yl)(2′-(trifluoromethyl)biphenyl-4-yl)methanone;
(S)-(4′-chlorobiphenyl-4-yl)(2-methyl-4-(pyrimidin-2-yl)piperazin-1-yl)methanone;
(5′-chloro-2′-fluorobiphenyl-4-yl)(4-(pyrimidin-2-yl)piperazin-1-yl)methanone;
(4-(5-methylthiophen-2-yl)phenyl)(1-(pyrimidin-2-yl)piperidin-4-yl)methanone;
biphenyl-4-yl(4-(4,6-dimethylpyrimidin-2-yl)piperazin-1-yl)methanone;
(S)-(2-methyl-4-(pyrimidin-2-yl)piperazin-1-yl)(4′-(trifluoromethyl)biphenyl-4-yl)methanone;
(S)-(2-benzyl-4-(pyrimidin-2-yl)piperazin-1-yl)(4′-chlorobiphenyl-4-yl)methanone;
biphenyl-4-yl(4-(pyridazin-3-yl)piperazin-1-yl)methanone;
(6-(4-methylthiophen-2-yl)pyridin-3-yl)(4-(pyrimidin-2-yl)piperazin-1-yl)methanone;
1-(2′,4′-difluorobiphenylcarbonyl)-4-(pyrimidin-2-yl)piperazine-2-carbonitrile;
(4′-chlorobiphenyl-4-yl)((2S,5S)-2,5-dimethyl-4-(pyrimidin-2-yl)piperazin-1-yl)methanone;
biphenyl-4-yl(2-tert-butyl-4-(pyrimidin-2-yl)piperazin-1-yl)methanone;
(S)-biphenyl-4-yl(2-isopropyl-4-(pyrimidin-2-yl)piperazin-1-yl)methanone;
biphenyl-4-yl(2,6-dimethyl-4-(pyrimidin-2-yl)piperazin-1-yl)methanone;
(3′-chloro-2′-fluorobiphenyl-4-yl)(4-(pyrimidin-2-yl)piperazin-1-yl)methanone;
(4-(pyrimidin-2-yl)piperazin-1-yl)(6-(4-(trifluoromethyl)phenyl)pyridin-3-yl)methanone;
(4′-chloro-3′-methylbiphenyl-4-yl)(1-(pyrimidin-2-yl)piperidin-4-yl)methanone;
(3′-chloro-2-fluorobiphenyl-4-yl)(4-(pyrimidin-2-yl)piperazin-1-yl)methanone;
(2,6-dimethyl-4-(pyridin-2-yl)piperazin-1-yl)(4′-methylbiphenyl-4-yl)methanone;
3′-chloro-4-(1-(pyrimidin-2-yl)piperidine-4-carbonyl)biphenyl-3-yl acetate;
biphenyl-4-yl(2-methyl-4-(pyridin-2-yl)piperazin-1-yl)methanone;
1-(biphenyl-4-ylmethyl)-4-(pyrimidin-2-yl)piperazin-2-one;
(3′,4′-dichlorobiphenyl-4-yl)(4-(pyrimidin-2-yl)piperazin-1-yl)methanone;
(3′-chlorobiphenyl-4-yl)(8-(pyrimidin-2-yl)-8-azabicyclo[3.2.1]octan-3-yl)methanol;
(S)-biphenyl-4-yl(2-isobutyl-4-(pyrimidin-2-yl)piperazin-1-yl)methanone;
(3′-chlorobiphenyl-4-yl)(8-(pyrimidin-2-yl)-8-azabicyclo[3.2.1]octan-3-yl)methanone;
(S)-(4′-chlorobiphenyl-4-yl)(2-isopropyl-4-(pyrimidin-2-yl)piperazin-1-yl)methanone;
(4′-methylbiphenyl-4-yl)(4-(pyridin-2-yl)piperazin-1-yl)methanone;
(S)-(2-isopropyl-4-(pyrimidin-2-yl)piperazin-1-yl)(4′-(trifluoromethyl)biphenyl-4-yl)methanone;
(3′-chlorobiphenyl-4-yl)(1-(pyrimidin-2-yl)piperidin-4-yl)methanol;
(S)-(2-isobutyl-4-(pyrimidin-2-yl)piperazin-1-yl)(4′-(trifluoromethyl)biphenyl-4-yl)methanone;
(S)-(4′-chlorobiphenyl-4-yl)(2-isobutyl-4-(pyrimidin-2-yl)piperazin-1-yl)methanone;
(3′-chlorobiphenyl-4-yl)(1-(pyrimidin-2-yl)pyrrolidin-3-yl)methanone;
(2′,4′-difluorobiphenyl-4-yl)(8-(pyrimidin-2-yl)-8-azabicyclo[3.2.1]octan-3-yl)methanone;
4′-(4-(pyridin-2-yl)piperazine-1-carbonyl)biphenyl-4-carbonitrile;
(4-(pyrimidin-2-yl)-1,4-diazepan-1-yl)(3′-(trifluoromethyl)biphenyl-4-yl)methanone;
methyl 1-(5′-chloro-2′-fluorobiphenylcarbonyl)-4-(pyrimidin-2-yl)piperazine-2-carboxylate;
(4-(benzo[d]oxazol-2-yl)piperazin-1-yl)(4′-chlorobiphenyl-4-yl)methanone;
(3′-chlorobiphenyl-4-yl)(4-(thiazol-2-yl)piperazin-1-yl)methanone;
(4-(5-chlorothiophen-2-yl)phenyl)(1-(pyrimidin-2-yl)piperidin-4-yl)methanone;
1-(5′-chloro-2′-fluorobiphenylcarbonyl)-4-(pyrimidin-2-yl)piperazine-2-carbonitrile;
(4-phenylthiophen-2-yl)(4-(pyrimidin-2-yl)piperazin-1-yl)methanone;
biphenyl-4-yl(4-(pyrimidin-2-yl)-3,4-dihydroquinoxalin-1(2H)-yl)methanone;
(5′-chloro-2′-fluorobiphenyl-4-yl)(8-(pyrimidin-2-yl)-8-azabicyclo[3.2.1]octan-3-yl)methanol;
(5-phenylfuran-2-yl)(4-(pyrimidin-2-yl)piperazin-1-yl)methanone;
(4′-chlorobiphenyl-4-yl)(5-(pyrimidin-2-yl)hexahydropyrrolo[3,4-c]pyrrol-2(1H)-yl)methanone;
4-(4′-chlorobiphenyl-4-yl)-1-(pyrimidin-2-yl)piperidin-4-ol;
(5-(pyrimidin-2-yl)hexahydropyrrolo[3,4-c]pyrrol-2(1H)-yl)(4′-(trifluoromethyl)biphenyl-4-yl)methanone;
biphenyl-4-yl(5-(pyridin-2-yl)hexahydropyrrolo[3,4-c]pyrrol-2(1H)-yl)methanone;
(3′-chlorobiphenyl-4-yl)(5-(pyrimidin-2-yl)hexahydropyrrolo[3,4-c]pyrrol-2(1H)-yl)methanone;
biphenyl-4-yl((2S,5S)-2,5-dimethyl-4-(pyrimidin-2-yl)piperazin-1-yl)methanone;
1-((3′-chlorobiphenyl-4-yl)methyl)-N,N-dimethyl-4-(pyrimidin-2-yl)piperazine-2-carboxamide;
(2′,4′-difluorobiphenyl-4-yl)(3-methyl-1-(pyrimidin-2-yl)piperidin-4-yl)methanone;
(4-(benzo[d]thiazol-2-yl)piperazin-1-yl)(biphenyl-4-yl)methanone;
biphenyl-4-yl(4-(quinolin-2-yl)piperazin-1-yl)methanone;
4-(biphenyl-4-yl)-1-(pyrimidin-2-yl)piperidin-4-ol;
4′-chloro-N-methyl-N-(2-(methyl(pyrimidin-2-yl)amino)ethyl)biphenyl-4-carboxamide;
2-(biphenyl-4-yl)-1-(4-(pyrimidin-2-yl)piperazin-1-yl)ethanone;
(S)—N-(7-tert-butyl-5-(2-(pyrrolidin-1-ylmethyl)pyrrolidine-1-carbonyl)-7H-pyrrolo[2,3-d]pyrimidin-2-yl)-4-methylbenzamide;
7-tert-butyl-2-(3,4-dimethylbenzamido)-N-(pyridin-3-ylmethyl)-7H-pyrrolo[2,3-d]pyrimidine-5-carboxamide;
7-tert-butyl-2-(4-methylbenzamido)-N-(pyridin-4-ylmethyl)-7H-pyrrolo[2,3-d]pyrimidine-5-carboxamide;
N-(7-tert-butyl-5-(pyrrolidine-1-carbonyl)-7H-pyrrolo[2,3-d]pyrimidin-2-yl)-4-methylbenzamide;
7-tert-butyl-N-ethyl-2-(4-methylbenzamido)-7H-pyrrolo[2,3-d]pyrimidine-5-carboxamide;
7-tert-butyl-2-(4-methylbenzamido)-N-((6-(trifluoromethyl)pyridin-3-yl)methyl)-7H-pyrrolo[2,3-d]pyrimidine-5-carboxamide;
7-tert-butyl-N-methyl-2-(4-methylbenzamido)-7H-pyrrolo[2,3-d]pyrimidine-5-carboxamide;
7-tert-butyl-N-isobutyl-2-(4-methylbenzamido)-7H-pyrrolo[2,3-d]pyrimidine-5-carboxamide;
7-tert-butyl-N-(2-(dimethylamino)ethyl)-2-(4-methylbenzamido)-7H-pyrrolo[2,3-d]pyrimidine-5-carboxamide;
7-tert-butyl-2-(4-methylbenzamido)-N-(pyridin-3-ylmethyl)-7H-pyrrolo[2,3-d]pyrimidine-5-carboxamide;
N-isopropyl-2-(4-methylbenzamido)-7-propyl-7H-pyrrolo[2,3-d]pyrimidine-5-carboxamide;
7-tert-butyl-2-(3-fluoro-4-methylbenzamido)-N-((6-(trifluoromethyl)pyridin-3-yl)methyl)-7H-pyrrolo[2,3-d]pyrimidine-5-carboxamide;
7-tert-butyl-2-(4-ethylbenzamido)-N-((6-(trifluoromethyl)pyridin-3-yl)methyl)-7H-pyrrolo[2,3-d]pyrimidine-5-carboxamide;
7-tert-butyl-2-(4-ethylbenzamido)-N-isopropyl-7H-pyrrolo[2,3-d]pyrimidine-5-carboxamide;
7-tert-butyl-N-isopropyl-2-(4-isopropylbenzamido)-7H-pyrrolo[2,3-d]pyrimidine-5-carboxamide;
7-tert-butyl-N-(2-ethoxyethyl)-2-(4-isopropylbenzamido)-7H-pyrrolo[2,3-d]pyrimidine-5-carboxamide;
7-tert-butyl-2-(4-isopropylbenzamido)-N-(pyridin-3-ylmethyl)-7H-pyrrolo[2,3-d]pyrimidine-5-carboxamide;
7-isobutyl-N-isopropyl-2-(4-methylbenzamido)-7H-pyrrolo[2,3-d]pyrimidine-5-carboxamide;
7-tert-butyl-2-(4-ethylbenzamido)-N-(2-methoxyethyl)-7H-pyrrolo[2,3-d]pyrimidine-5-carboxamide;
7-tert-butyl-2-(3-fluoro-4-methylbenzamido)-N-(2-methoxyethyl)-7H-pyrrolo[2,3-d]pyrimidine-5-carboxamide;
7-tert-butyl-2-(3-fluoro-4-methylbenzamido)-N-(pyridin-3-ylmethyl)-7H-pyrrolo[2,3-d]pyrimidine-5-carboxamide;
7-tert-butyl-N-(2-ethoxyethyl)-2-(3-fluoro-4-methylbenzamido)-7H-pyrrolo[2,3-d]pyrimidine-5-carboxamide;
7-tert-butyl-N-ethyl-2-(4-ethylbenzamido)-7H-pyrrolo[2,3-d]pyrimidine-5-carboxamide;
7-tert-butyl-2-(4-ethylbenzamido)-N-isobutyl-7H-pyrrolo[2,3-d]pyrimidine-5-carboxamide;
7-tert-butyl-N-cyclopropyl-2-(4-ethylbenzamido)-7H-pyrrolo[2,3-d]pyrimidine-5-carboxamide;
7-tert-butyl-2-(4-ethylbenzamido)-N-(pyridin-3-ylmethyl)-7H-pyrrolo[2,3-d]pyrimidine-5-carboxamide;
7-tert-butyl-2-(3-fluoro-4-methylbenzamido)-N-isobutyl-7H-pyrrolo[2,3-d]pyrimidine-5-carboxamide;
7-tert-butyl-2-(3-fluoro-4-methylbenzamido)-N-propyl-7H-pyrrolo[2,3-d]pyrimidine-5-carboxamide;
7-tert-butyl-2-(4-ethylbenzamido)-N-propyl-7H-pyrrolo[2,3-d]pyrimidine-5-carboxamide;
7-tert-butyl-N-isopropyl-4-methyl-2-(4-methylbenzamido)-7H-pyrrolo[2,3-d]pyrimidine-5-carboxamide;
7-tert-butyl-N-isopropyl-2-(4-methylbenzamido)-7H-pyrrolo[2,3-d]pyrimidine-5-carboxamide;
7-tert-butyl-N-cyclopropyl-2-(3-fluoro-4-methylbenzamido)-7H-pyrrolo[2,3-d]pyrimidine-5-carboxamide;
7-tert-butyl-N-(2-methoxyethyl)-2-(4-methylbenzamido)-7H-pyrrolo[2,3-d]pyrimidine-5-carboxamide;
7-tert-butyl-N-cyclopropyl-2-(4-methylbenzamido)-7H-pyrrolo[2,3-d]pyrimidine-5-carboxamide;
7-tert-butyl-N-(2-ethoxyethyl)-2-(4-ethylbenzamido)-7H-pyrrolo[2,3-d]pyrimidine-5-carboxamide;
7-tert-butyl-N-ethyl-2-(3-fluoro-4-methylbenzamido)-7H-pyrrolo[2,3-d]pyrimidine-5-carboxamide;
7-tert-butyl-N-(2-ethoxyethyl)-2-(4-methylbenzamido)-7H-pyrrolo[2,3-d]pyrimidine-5-carboxamide;
7-tert-butyl-N-(1-methoxypropan-2-yl)-2-(4-methylbenzamido)-7H-pyrrolo[2,3-d]pyrimidine-5-carboxamide;
7-tert-butyl-2-(4-propylbenzamido)-N-(pyridin-3-ylmethyl)-7H-pyrrolo[2,3-d]pyrimidine-5-carboxamide;
7-isobutyl-2-(4-methylbenzamido)-N-((6-(trifluoromethyl)pyridin-3-yl)methyl)-7H-pyrrolo[2,3-d]pyrimidine-5-carboxamide;
N-isopropyl-2-(4-methylbenzamido)-7-tert-pentyl-7H-pyrrolo[2,3-d]pyrimidine-5-carboxamide;
2-(4-methylbenzamido)-7-tert-pentyl-N-((6-(trifluoromethyl)pyridin-3-yl)methyl)-7H-pyrrolo[2,3-d]pyrimidine-5-carboxamide;
2-(4-methylbenzamido)-7-propyl-N-((6-(trifluoromethyl)pyridin-3-yl)methyl)-7H-pyrrolo[2,3-d]pyrimidine-5-carboxamide;
7-tert-butyl-2-(3-fluoro-4-methylbenzamido)-N-isopropyl-7H-pyrrolo[2,3-d]pyrimidine-5-carboxamide;
N-(2-ethoxyethyl)-2-(4-methylbenzamido)-7-tert-pentyl-7H-pyrrolo[2,3-d]pyrimidine-5-carboxamide;
7-tert-butyl-2-(3,4-dimethylbenzamido)-N-isopropyl-7H-pyrrolo[2,3-d]pyrimidine-5-carboxamide;
7-tert-butyl-2-(3,4-dimethylbenzamido)-N-(2-ethoxyethyl)-7H-pyrrolo[2,3-d]pyrimidine-5-carboxamide;
4-methyl-N-(7-tert-pentyl-5-(pyrrolidine-1-carbonyl)-7H-pyrrolo[2,3-d]pyrimidin-2-yl)benzamide;
2-(4-methylbenzamido)-7-tert-pentyl-N-(pyridin-3-ylmethyl)-7H-pyrrolo[2,3-d]pyrimidine-5-carboxamide;
7-tert-butyl-2-(4-methylbenzamido)-N-(pyridin-2-ylmethyl)-7H-pyrrolo[2,3-d]pyrimidine-5-carboxamide;
7-tert-butyl-N,N-dimethyl-2-(4-methylbenzamido)-7H-pyrrolo[2,3-d]pyrimidine-5-carboxamide;
7-tert-butyl-2-(4-methylbenzamido)-N-propyl-7H-pyrrolo[2,3-d]pyrimidine-5-carboxamide;
N-cyclopropyl-2-(4-methylbenzamido)-7-tert-pentyl-7H-pyrrolo[2,3-d]pyrimidine-5-carboxamide;
7-tert-butyl-N-isopropyl-2-(4-propylbenzamido)-7H-pyrrolo[2,3-d]pyrimidine-5-carboxamide;
7-tert-butyl-N-(2-ethoxyethyl)-2-(4-propylbenzamido)-7H-pyrrolo[2,3-d]pyrimidine-5-carboxamide;
1-(cyclobutylmethyl)-N-cyclopropyl-6-(4-methylbenzamido)-1H-pyrazolo[3,4-b]pyridine-3-carboxamide;
(S)—N-(1-tert-butyl-3-(2-isobutylpyrrolidine-1-carbonyl)-1H-pyrazolo[3,4-b]pyridin-6-yl)-4-methylbenzamide;
1-tert-butyl-6-(4-methylbenzamido)-N-(pyridin-3-ylmethyl)-1H-pyrazolo[3,4-b]pyridine-3-carboxamide;
N-(1-tert-butyl-3-(2-(pyridin-2-yl)piperidine-1-carbonyl)-1H-pyrazolo[3,4-b]pyridin-6-yl)-4-methylbenzamide;
1-tert-butyl-N-isobutyl-6-(4-methylbenzamido)-1H-pyrazolo[3,4-b]pyridine-3-carboxamide;
N-(2-(1H-indol-3-yl)ethyl)-1-tert-butyl-6-(4-methylbenzamido)-1H-pyrazolo[3,4-b]pyridine-3-carboxamide;
6-(4-methylbenzamido)-1-propyl-N-(1-(pyridin-3-yl)ethyl)-1H-pyrazolo[3,4-b]pyridine-3-carboxamide;
1-benzyl-N-isopropyl-6-(4-methylbenzamido)-1H-pyrazolo[3,4-b]pyridine-3-carboxamide;
1-tert-butyl-6-(4-methylbenzamido)-N-(1-(pyridin-3-yl)ethyl)-1H-pyrazolo[3,4-b]pyridine-3-carboxamide;
N-(1-isobutyl-3-(pyrrolidine-1-carbonyl)-1H-pyrazolo[3,4-b]pyridin-6-yl)-4-methylbenzamide;
1-tert-butyl-6-(4-methylbenzamido)-N-(2-(pyridin-3-yl)ethyl)-1H-pyrazolo[3,4-b]pyridine-3-carboxamide;
1-tert-butyl-6-(4-methylbenzamido)-N-(pyridin-2-ylmethyl)-1H-pyrazolo[3,4-b]pyridine-3-carboxamide;
N-cyclopropyl-1-isobutyl-6-(4-methylbenzamido)-1H-pyrazolo[3,4-b]pyridine-3-carboxamide;
1-isobutyl-6-(4-methylbenzamido)-N-(1-(pyridin-3-yl)ethyl)-1H-pyrazolo[3,4-b]pyridine-3-carboxamide;
1-isopropyl-6-(4-methylbenzamido)-N-(1-(pyridin-3-yl)ethyl)-1H-pyrazolo[3,4-b]pyridine-3-carboxamide;
1-isopropyl-N,N-dimethyl-6-(4-methylbenzamido)-1H-pyrazolo[3,4-b]pyridine-3-carboxamide;
1-benzyl-N,N-dimethyl-6-(4-methylbenzamido)-1H-pyrazolo[3,4-b]pyridine-3-carboxamide;
1-tert-butyl-N-isopropyl-6-(6-methylnicotinamido)-1H-pyrazolo[3,4-b]pyridine-3-carboxamide;
1-benzyl-6-(4-methylbenzamido)-N-(2-(pyridin-2-yl)ethyl)-1H-pyrazolo[3,4-b]pyridine-3-carboxamide;
1-benzyl-6-(4-methylbenzamido)-N-(1-(pyridin-3-yl)ethyl)-1H-pyrazolo[3,4-b]pyridine-3-carboxamide;
N,N-dimethyl-6-(4-methylbenzamido)-1-propyl-1H-pyrazolo[3,4-b]pyridine-3-carboxamide;
6-(4-methylbenzamido)-1-propyl-N-(2-(pyridin-2-yl)ethyl)-1H-pyrazolo[3,4-b]pyridine-3-carboxamide;
N-(1-isopentyl-3-(pyrrolidine-1-carbonyl)-1H-pyrazolo[3,4-b]pyridin-6-yl)-4-methylbenzamide;
1-(cyclobutylmethyl)-6-(4-methylbenzamido)-N-pentyl-1H-pyrazolo[3,4-b]pyridine-3-carboxamide;
N-isopropyl-6-(4-methylbenzamido)-1-propyl-1H-pyrazolo[3,4-b]pyridine-3-carboxamide;
1-tert-butyl-N-isopropyl-6-(3-methylbenzamido)-1H-pyrazolo[3,4-b]pyridine-3-carboxamide;
N-isopropyl-6-(4-methylbenzamido)-1-(2,2,2-trifluoroethyl)-1H-pyrazolo[3,4-b]pyridine-3-carboxamide;
6-(4-methylbenzamido)-N-(2-(pyridin-2-yl)ethyl)-1-(2,2,2-trifluoroethyl)-1H-pyrazolo[3,4-b]pyridine-3-carboxamide;
6-(4-methylbenzamido)-N-(1-(pyridin-3-yl)ethyl)-1-(2,2,2-trifluoroethyl)-1H-pyrazolo[3,4-b]pyridine-3-carboxamide;
N-cyclopropyl-6-(4-methylbenzamido)-1-(2,2,2-trifluoroethyl)-1H-pyrazolo[3,4-b]pyridine-3-carboxamide;
6-(4-methylbenzamido)-1-phenethyl-N-(1-(pyridin-3-yl)ethyl)-1H-pyrazolo[3,4-b]pyridine-3-carboxamide;
N-cyclopropyl-6-(4-methylbenzamido)-1-phenethyl-1H-pyrazolo[3,4-b]pyridine-3-carboxamide;
1-tert-butyl-N-isopropyl-6-(4-methylbenzamido)-1H-pyrazolo[3,4-b]pyridine-3-carboxamide;
(S)—N-(1-tert-butyl-3-(2-(pyrrolidin-1-ylmethyl)pyrrolidine-1-carbonyl)-1H-pyrazolo[3,4-b]pyridin-6-yl)-4-methylbenzamide;
1-tert-butyl-6-(4-methylbenzamido)-N-(2-(pyridin-3-yl)ethyl)-1H-pyrazolo[3,4-b]pyridine-3-carboxamide;
1-tert-butyl-N-isobutyl-6-(4-methylbenzamido)-1H-pyrazolo[3,4-b]pyridine-3-carboxamide;
1-tert-butyl-6-(4-methylbenzamido)-N-(pentan-3-yl)-1H-pyrazolo[3,4-b]pyridine-3-carboxamide;
N-((1H-indol-3-yl)methyl)-1-tert-butyl-6-(4-methylbenzamido)-1H-pyrazolo[3,4-b]pyridine-3-carboxamide;
4-methyl-N-(1-phenethyl-3-(pyrrolidine-1-carbonyl)-1H-pyrazolo[3,4-b]pyridin-6-yl)benzamide;
N-(1-(cyclobutylmethyl)-3-(pyrrolidine-1-carbonyl)-1H-pyrazolo[3,4-b]pyridin-6-yl)-4-methylbenzamide;
N-cyclopropyl-1-isopentyl-6-(4-methylbenzamido)-1H-pyrazolo[3,4-b]pyridine-3-carboxamide;
1-tert-butyl-N-cyclopropyl-6-(4-methylbenzamido)-1H-pyrazolo[3,4-b]pyridine-3-carboxamide;
(R)—N-(1-tert-butyl-3-(2-((dimethylamino)methyl)pyrrolidine-1-carbonyl)-1H-pyrrolo[2,3-b]pyridin-6-yl)-4-methylbenzamide;
1-tert-butyl-N-isopropyl-6-(4-methylbenzamido)-1H-pyrrolo[2,3-b]pyridine-3-carboxamide;
1-tert-butyl-6-(4-methylbenzamido)-N-(pentan-3-yl)-1H-pyrrolo[2,3-b]pyridine-3-carboxamide;
1-tert-butyl-6-(4-methylbenzamido)-N-(pyridin-4-ylmethyl)-1H-pyrrolo[2,3-b]pyridine-3-carboxamide;
1-tert-butyl-N-ethyl-6-(4-methylbenzamido)-1H-pyrrolo[2,3-b]pyridine-3-carboxamide;
(S)-1-tert-butyl-N-(2-hydroxy-1-phenylethyl)-6-(4-methylbenzamido)-1H-pyrrolo[2,3-b]pyridine-3-carboxamide;
1-tert-butyl-N-(2-ethoxyethyl)-6-(4-methylbenzamido)-1H-pyrrolo[2,3-b]pyridine-3-carboxamide;
N-benzyl-1-tert-butyl-6-(4-methylbenzamido)-1H-pyrrolo[2,3-b]pyridine-3-carboxamide;
1-tert-butyl-6-(4-methylbenzamido)-N-((3-methylpyridin-2-yl)methyl)-1H-pyrrolo[2,3-b]pyridine-3-carboxamide;
(S)—N-(1-methoxypropan-2-yl)-6-(4-methylbenzamido)-1-neopentyl-1H-pyrrolo[2,3-b]pyridine-3-carboxamide;
1-tert-butyl-6-(4-methylbenzamido)-N-(1-(pyridin-3-yl)ethyl)-1H-pyrrolo[2,3-b]pyridine-3-carboxamide;
1-tert-butyl-N,N-dimethyl-6-(4-methylbenzamido)-1H-pyrrolo[2,3-b]pyridine-3-carboxamide;
(S)—N-sec-butyl-1-tert-butyl-6-(4-methylbenzamido)-1H-pyrrolo[2,3-b]pyridine-3-carboxamide;
1-tert-butyl-6-(4-methylbenzamido)-N-(1-(pyridin-4-yl)ethyl)-1H-pyrrolo[2,3-b]pyridine-3-carboxamide;
(S)-1-tert-butyl-N-(1-methoxypropan-2-yl)-6-(4-methylbenzamido)-1H-pyrrolo[2,3-b]pyridine-3-carboxamide;
1-tert-butyl-N,N-diethyl-6-(4-methylbenzamido)-1H-pyrrolo[2,3-b]pyridine-3-carboxamide;
N-(1-tert-butyl-3-(pyrrolidine-1-carbonyl)-1H-pyrrolo[2,3-b]pyridin-6-yl)-4-methylbenzamide;
1-tert-butyl-N-isobutyl-6-(4-methylbenzamido)-1H-pyrrolo[2,3-b]pyridine-3-carboxamide;
1-tert-butyl-N-cyclobutyl-6-(4-methylbenzamido)-1H-pyrrolo[2,3-b]pyridine-3-carboxamide;
(R)-1-tert-butyl-6-(4-methylbenzamido)-N-(3-methylbutan-2-yl)-1H-pyrrolo[2,3-b]pyridine-3-carboxamide;
1-tert-butyl-N-(1-methoxypropan-2-yl)-6-(4-methylbenzamido)-1H-pyrrolo[2,3-b]pyridine-3-carboxamide;
(R)-1-tert-butyl-6-(N,4-dimethylbenzamido)-N-(1-methoxypropan-2-yl)-1H-pyrrolo[2,3-b]pyridine-3-carboxamide;
1-tert-butyl-N-(furan-2-ylmethyl)-6-(4-methylbenzamido)-1H-pyrrolo[2,3-b]pyridine-3-carboxamide;
(R)-1-tert-butyl-N-(hexan-2-yl)-6-(4-methylbenzamido)-1H-pyrrolo[2,3-b]pyridine-3-carboxamide;
1-tert-butyl-6-(4-methylbenzamido)-N-(oxazol-2-ylmethyl)-1H-pyrrolo[2,3-b]pyridine-3-carboxamide;
1-tert-butyl-N-cyclopropyl-6-(4-methylbenzamido)-1H-pyrrolo[2,3-b]pyridine-3-carboxamide;
1-tert-butyl-6-(4-methylbenzamido)-N-(pyridin-2-ylmethyl)-1H-pyrrolo[2,3-b]pyridine-3-carboxamide;
(R)—N-(1-tert-butyl-3-(2-(methoxymethyl)pyrrolidine-1-carbonyl)-1H-pyrrolo[2,3-b]pyridin-6-yl)-4-methylbenzamide;
1-tert-butyl-6-(4-methylbenzamido)-N-propyl-1H-pyrrolo[2,3-b]pyridine-3-carboxamide;
N-(2-tert-butoxyethyl)-1-tert-butyl-6-(4-methylbenzamido)-1H-pyrrolo[2,3-b]pyridine-3-carboxamide;
1-tert-butyl-6-(4-methylbenzamido)-N-(thiazol-2-ylmethyl)-1H-pyrrolo[2,3-b]pyridine-3-carboxamide;
1-tert-butyl-N-(cyclopropylmethyl)-6-(4-methylbenzamido)-1H-pyrrolo[2,3-b]pyridine-3-carboxamide;
N-(1-tert-butyl-3-(morpholine-4-carbonyl)-1H-pyrrolo[2,3-b]pyridin-6-yl)-4-methylbenzamide;
1-tert-butyl-6-(4-methylbenzamido)-N-(tetrahydro-2H-pyran-4-yl)-1H-pyrrolo[2,3-b]pyridine-3-carboxamide;
1-tert-butyl-6-(4-methylbenzamido)-N-(1-methylpiperidin-4-yl)-1H-pyrrolo[2,3-b]pyridine-3-carboxamide;
(R)-1-tert-butyl-6-(4-ethylbenzamido)-N-(1-methoxypropan-2-yl)-1H-pyrrolo[2,3-b]pyridine-3-carboxamide; and
(R)-6-(4-ethylbenzamido)-1-isobutyl-N-(1-methoxypropan-2-yl)-1H-pyrrolo[2,3-b]pyridine-3-carboxamide.

Preferred compounds are potent proline transporter inhibitors. Particular potent proline transporter inhibitors have a PTIC₅₀ of less than about 150, 125, 100, 75, 50 or 25 nM.

Some compounds inhibit the murine Na⁺-dependent proline transporter, as determined by the method described in the Examples below, with an IC₅₀ of less than about 150, 125, 100, 75, 50 or 25 nM.

Some compounds do not significantly inhibit the dopamine transporter. For example, some potent proline transporter inhibitors inhibit the dopamine transporter with an IC₅₀ of greater than about 0.5, 1, 2.5, 5, or 10 μM as determined using the assay described in the Examples below.

Some compounds do not significantly inhibit the glycine transporter. For example, some potent proline transporter inhibitors inhibit the glycine transporter with an IC₅₀ of greater than about 0.5, 1, 2.5, 5, or 10 μM as determined using the assay described in the Examples below.

### 5.3. Preparation of Compounds

Compounds of the invention may be obtained or prepared using synthetic methods known in the art, as well as those described herein. For example, various piperazine-based compounds encompassed by formula I can be prepared according to the general approach shown in Scheme I:

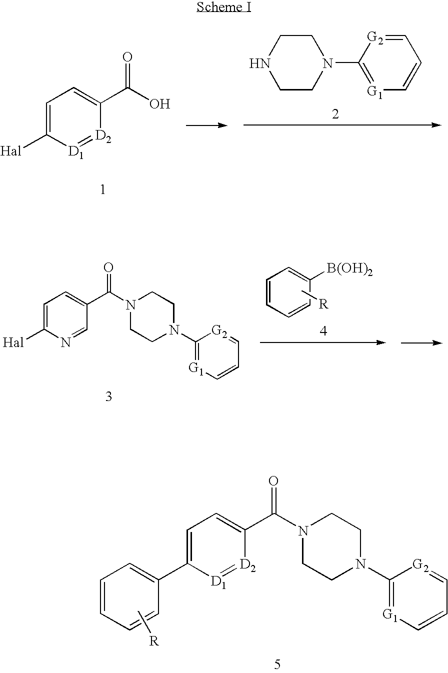

In this approach, a compound of formula 1 (D₁ and D₂ are defined herein) is contacted with a compound of formula 2 (G₁ and G₂ are defined herein) under suitable conditions to provide a compound of formula 3. Suitable conditions include, for example, EDCl, HOBt, and Hunig's base in DMF. Compound 3 is then contacted with compound 4 under suitable conditions to provide a compound of formula 5. Suitable conditions include, for example, Pd(Ph₃P)₄, K₃PO₄, DME, water and heat.

Various piperidine-based compounds encompassed by formula I can be prepared according to the general approach shown below in Scheme II:

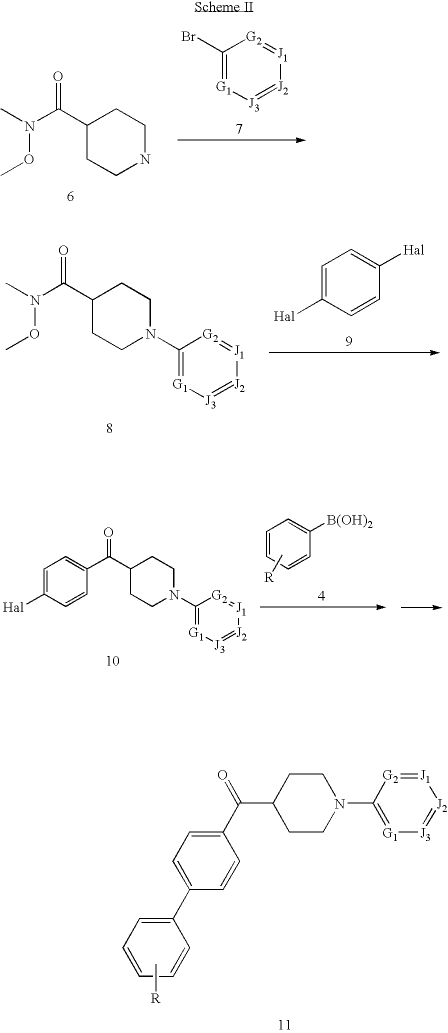

In this approach, a compound of formula 6 (e.g., as a TFA salt) is contacted with a compound of formula 7 (G₁, G₂, J₁, J₂ and J₃ are defined herein) under suitable conditions to provide compound 8. Suitable conditions include, for example, TEA and heat. Compound 8 is then contacted with compound 9 under suitable conditions to provide compound 10. Here, suitable conditions include, for example, n-BuLi in THF. Compound 10 is then contacted with a compound of formula 4 to provide the final compound, 11. Here, suitable conditions include, for example, Pd(Ph₃P)₄, K₃PO₄, DME, water and heat.

If desired, compounds of formula 11 can be reduced under suitable conditions (e.g., sodium borohydride) to provide compounds of formula 12, as shown below in Scheme III:

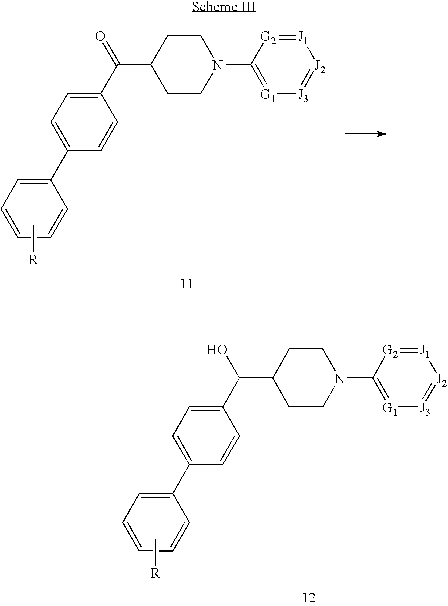

Compounds encompassed by formula I containing an ether link can be prepared by routes such as that shown in Scheme IV:

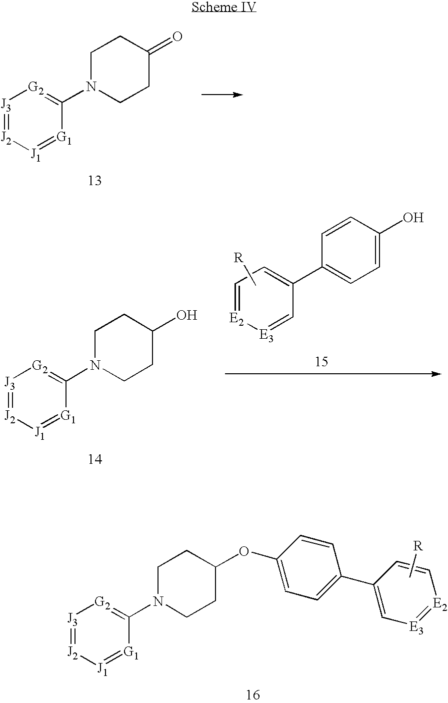

In this approach, a compound of formula 13 is reduced (e.g., with sodium borohydride) to provide compound 14, which is then coupled under suitable reaction conditions with a compound of formula 15 to provide compound 16. Suitable reaction conditions include, for example, PPh₃ and DEAD in THF.

Compounds encompassed by formula I containing a methylene link can be prepared by routes such as that shown in Scheme V:

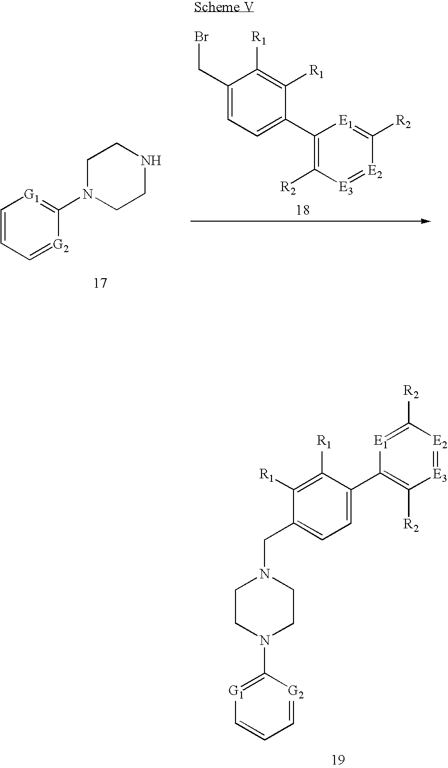

In this approach, a compound of formula 17 is contacted with compound 18 under suitable reaction conditions to provide compound 19. Suitable reaction conditions include, for example, potassium carbonate in DMF.

Pyrrolopyrimidine compounds encompassed by formula III can generally be prepared as shown below in Scheme VI:

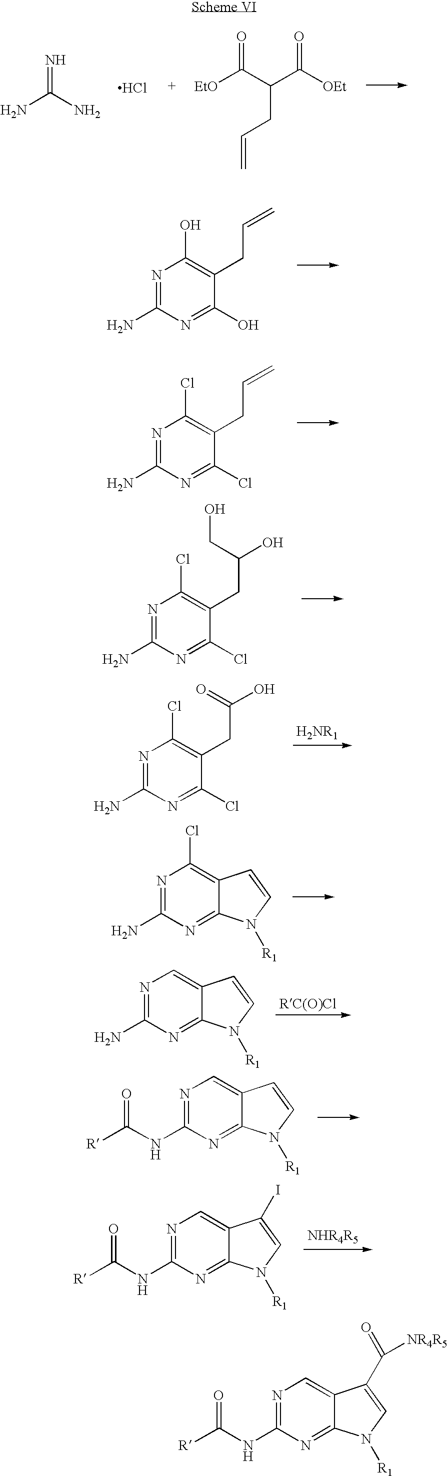

In this approach, 5-allyl-2-amino-pyrimidine-4,6-diol is prepared by the reaction of guanidine with 2-allyl-malonic acid diethyl ester (e.g., in base). The diol is converted to the corresponding di-chloride (e.g., with POCl₃), which is then oxidized (e.g., with OsO₄) to afford 3-(2-amino-4,6-dichloro-pyrimidin-5-yl)-propane-1,2-diol, which is subsequently converted to (2-amino-4,6-dichloro-pyrimidin-5-yl)-acetaldehyde (e.g., with Pb(OAc)₄). The aldehylde is cyclized to obtain a substituted 4-chloro-pyrrolopyrimidine. The chlorine is removed (e.g., with H₂, Pd/C), and the resulting compound is reacted with the desired acid chloride, then iodinated, and finally reacted with the desired amine to obtain the final product.

Pyrrolopyridine compounds encompassed by formula IV can generally be prepared as shown below in Scheme VII:

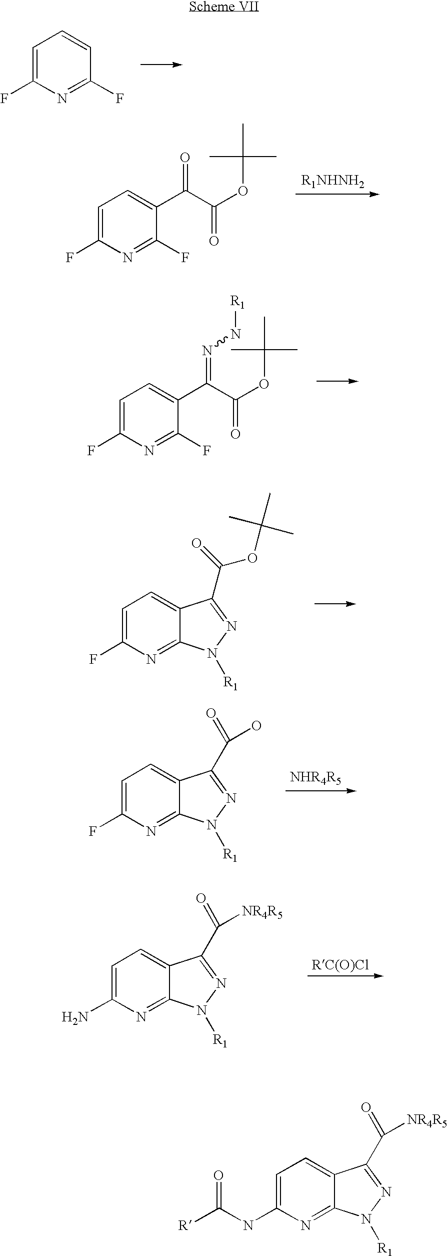

In this approach, 2,6-difluoro-pyridine is reacted with oxalic acid di-tert-butyl ester to afford (2,6-difluoro-pyridin-3-yl)-oxo-acetic acid tert-butyl ester. This is converted to the desired (2,6-difluoro-pyridin-3-yl)-hydrazono-acetic acid tert-butyl ester, which is subsequently cyclized to afford the corresponding 6-fluoro-1H-pyrazolo[3,4-b]pyridine-3-carboxylic acid tert-butyl ester. The tert-butyl ester is removed to yield the corresponding acid, which is reacted with the appropriate amine to afford the desired amide. The amide is reacted with the desired acid chloride to obtain the final product.

Pyrazolopyrimidine compounds encompassed by formula V can generally be prepared as shown in Scheme VIII:

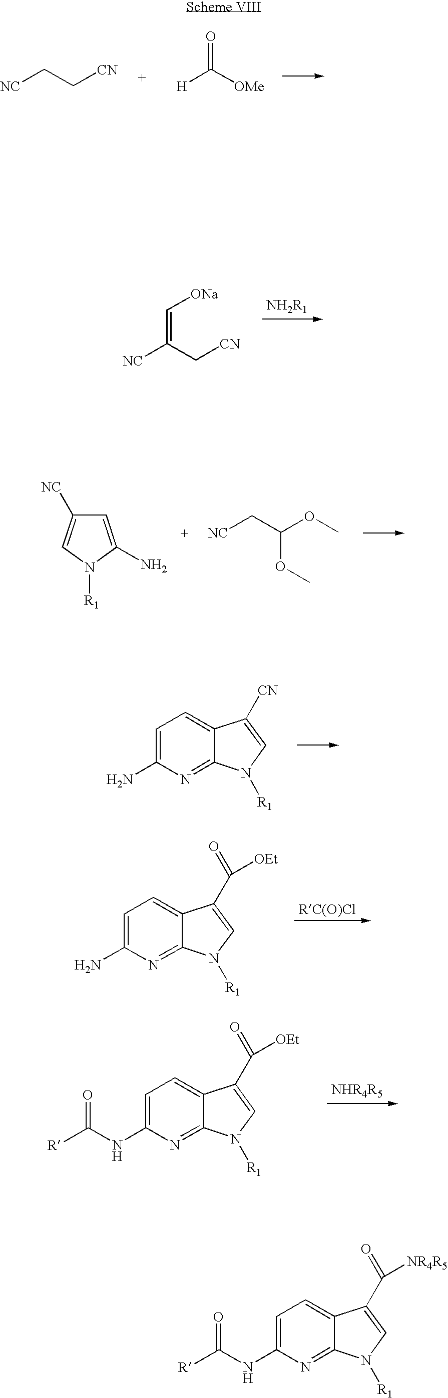

In this approach, succinonitrile is reacted with formic acid methyl ester to afford 2,3-dicyano-propen-1-ol sodium, with is reacted with an amine to yield the desired N-substituted 5-amino-1H-pyrrole-3-carbonitrile. The pyrrole is reacted with 3,3-dimethoxy-propionitrile in acidic conditions to afford a 6-amino-1H-pyrrolo[2,3-b]pyridine-3-carbonitrile, which is converted into the corresponding ethyl ester (e.g., with H₂SO₄ in EtOH). The ethyl ester is next reacted with the desired acid chloride, and finally reacted with the desired amine to yield the final product.

Some specific reaction conditions that can be used in the various synthetic schemes shown above are provided in the Examples, below.

### 5.4. Nucleic Acid Modulators

Nucleic acid based modulators of SLC6A7 expression or activity may also be used in methods of the invention. Nucleic acid modulators of SLC6A7 can be aptamers, polynucleotides or oligonucleotides that encode a portion of SLC6A7 or, when corresponding to the non-coding strand, act as SLC6A7 antisense molecules that modulate SLC6A7 gene expression. With respect to SLC6A7 gene regulation, polynucleotides and oligonucleotides that target SLC6A7 expression may be used to regulate one or more of the biological functions associated with SLC6A7. Further, such SLC6A7-targeted polynucleotides and oligonucleotides can be used as part of ribozyme and/or triple helix sequences that may also useful for modulating SLC6A7 gene expression or activity.

Nucleic acid modulators of SLC6A7 expression can comprise an RNA molecule that reduces expression of a target nucleic acid by a RNA interference (RNAi)-based mechanism. Examples of RNA molecules suitable for RNAi include short interfering RNAs (siRNAs), microRNAs, tiny non-coding RNAs (tncRNAs), and small modulatory RNA (smRNA). See, e.g., Novina et al., Nature 430:161-164 (2004).

Inhibitory oligonucleotides may comprise at least one modified base moiety, such as 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethyl-aminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5N-methoxycarboxy-methyluracil, 5-methoxyuracil, 2-methylthio-N6-isopentenyladenine, wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid, 3-(3-amino-3-N-2-carboxypropyl)uracil and 2,6-diaminopurine.

Inhibitory oligonucleotides may also comprise at least one modified sugar moiety, such as arabinose, 2-fluoroarabinose, xylulose, and hexose.

Inhibitory oligonucleotides may also comprise at least one modified phosphate backbone, such as a phosphorothioate, a phosphorodithioate, a phosphoramidothioate, a phosphoramidate, a phosphordiamidate, a methylphosphonate, an alkyl phosphotriester, or a formacetal or analog thereof.

In one embodiment, the inhibitory oligonucleotide is an α-anomeric oligonucleotide. An α-anomeric oligonucleotide forms specific double-stranded hybrids with complementary RNA in which, contrary to the usual β-units, the strands run parallel to each other. Gautier et al., Nucl. Acids Res. 15:6625-6641 (1987). The oligonucleotide can also be a 2N—O-methylribonucleotide (Inoue et al., Nucl. Acids Res. 15:6131-6148 (1987)) or a chimeric RNA-DNA analogue (Inoue et al, FEBS Lett. 215:327-330 (1987)). Alternatively, double-stranded RNA may be used to disrupt the expression and function of SLC6A7.

The activity of an inhibitory oligonucleotide or nucleic acid, such as an antisense DNA molecule or a siRNA, is often affected by the secondary structure of the target mRNA. See, e.g., Vickers et al., J. Biol. Chem. 278:7108-7118 (2003). Thus, inhibitory nucleic acids can be selected that are complementary to a region of a target mRNA that is available for interacting with an inhibitory oligonucleotide. A suitable region of a target mRNA can be identified by performing a “gene walk,” e.g., by empirically testing a number of oligonucleotides for their ability to interact with regions along a target mRNA and/or to reduce target mRNA expression. See, e.g., Vickers et al., supra; Hill et al., Am. J. Respir. Cell Mol. Biol. 21:728-737 (1999). Alternatively, a suitable region of a target mRNA can be identified using a mRNA secondary structure prediction program or related algorithm to identify regions of the target mRNA that do not hybridize to any other regions of the target mRNA. See, e.g., Hill et al., supra. A combination of both of the above methods can also be used to identify a suitable region of a target mRNA.

### 5.5. Methods of Treatment

This invention encompasses methods of treating, preventing and managing cognitive impairment associated with, or caused by, various diseases and disorders, including Attention-Deficit/Hyperactivity Disorder (ADD/ADHD), Down syndrome, Fragile X syndrome, Huntington's disease, Parkinson's disease, and schizophrenia.

The invention also encompasses methods of treating, preventing and managing age-associated memory impairment.

The invention also encompasses methods of treating, preventing and managing dementia associated with metabolic-toxic, structural and/or infectious causes.

Metabolic-toxic causes of dementia include: anoxia; B₁₂ deficiency; chronic drug, alcohol or nutritional abuse; folic acid deficiency; hypercalcemia associated with hyperparathyroidism; hypoglycemia; hypothyroidism; organ system failure (e.g., hepatic, respiratory, or uremic encephalopathy); and pellagra.

Structural causes of dementia include: amyotrophic lateral sclerosis; brain trauma (e.g., chronic subdural hematoma, dementia pugilistica); brain tumors; cerebellar degeneration; communicating hydrocephalus; irradiation to frontal lobes; multiple sclerosis; normal-pressure hydrocephalus; Pick's disease; progressive multifocal leukoencephalopathy; progressive supranuclear palsy; surgery; vascular disease (e.g., multi-infarct dementia); and Wilson's disease.

Infectious causes of dementia include: bacterial endocarditis; Creutzfeldt-Jakob disease; Gerstmann-Sträussler-Scheinker disease; HIV-related disorders; neurosyphilis; tuberculous and fungal meningitis; and viral encephalitis.

One embodiment encompasses methods wherein proline transporter activity in the patient is decreased. In particular methods, the activity is decreased by administering to the patient an effective amount of a compound that inhibits the proline transporter (e.g., a potent proline transporter inhibitor). In others, the activity is decreased by administering to the patient an effective amount of a compound that interferes with the expression of the gene that encodes the proline transporter (e.g., SLC6A7).

Another embodiment encompasses methods which comprise administering to the patient an effective amount of a compound that inhibits the proline transporter. In a particular method, the compound is a potent proline transporter inhibitor.

Another embodiment encompasses a method of inhibiting a proline transporter, which comprises contacting a proline transporter (in vitro or in vivo) with a sufficient amount of a compound of the invention.

In each of the various methods of the invention, preferred proline transporters are encoded by the human gene SLC6A7, the murine ortholog thereof, or a nucleic acid molecule that encodes a proline transporter and that hybridizes under standard conditions to the full length of either. The most preferred proline transporter is encoded by the human gene SLC6A7.

### 5.6. Pharmaceutical Compositions

This invention encompasses pharmaceutical compositions and dosage forms comprising compounds of the invention as their active ingredients. Pharmaceutical compositions and dosage forms of this invention may optionally contain one or more pharmaceutically acceptable carriers or excipients. Certain pharmaceutical compositions are single unit dosage forms suitable for oral, topical, mucosal (e.g., nasal, pulmonary, sublingual, vaginal, buccal, or rectal), parenteral (e.g., subcutaneous, intravenous, bolus injection, intramuscular, or intraarterial), or transdermal administration to a patient. Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The formulation should suit the mode of administration. For example, oral administration may require enteric coatings to protect the active ingredient from degradation within the gastrointestinal tract. In another example, the active ingredient may be administered in a liposomal formulation to shield it from degradative enzymes, facilitate transport in circulatory system, and/or effect delivery across cell membranes to intracellular sites.

The composition, shape, and type of dosage forms of the invention will typically vary depending on their use and active ingredients. For example, a dosage form used in the acute treatment of a disease may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease. These and other ways in which specific dosage forms encompassed by this invention will vary from one another will be readily apparent to those skilled in the art. See, e.g., Remington's Pharmaceutical Sciences, 18th ed., Mack Publishing, Easton Pa. (1990).

Nucleic acid modulators of SLC6A7 can be suitably formulated and administered by any number of methods known to those skilled in the art including, but not limited to, gene delivery, electroporation, inhalation, intranasal introduction, subcutaneous, intravenous, intraperitoneal, intramuscular, intrathecal injection, or intracranial injection.

### 6. EXAMPLES

### 6.1. SLC6A7-Deficient Mice

To determine the effect of inhibiting the Na⁺-dependent proline transporter, mice homozygous for a genetically engineered mutation in the murine ortholog of the human SLC6A7 gene (“knockout” or “KO” mice) were generated using correspondingly mutated ES cell clones from the OMNIBANK collection of mutated murine ES cell clones (see generally U.S. Pat. No. 6,080,576).

Mice that were heterozygous, homozygous, or wildtype for the mutated allele were produced by breeding heterozygous animals capable of germline transmission of the mutant allele. The mutated allele assorted according to standard Mendelian genetics. The mice were subjected to a battery of medical and behavioral tests, including those described below.

### 6.1.1. Trace Conditioning

Trace aversive conditioning measures a form of classical conditioning with temporal separation between the end of a conditioned stimulus (CS) (in this case an 80 db tone) and the onset of an unconditioned stimulus (US) (in this case a 0.7 mA electric current) that are separated by a temporal “trace” (approximately 30 seconds). This assay measures higher-order learning (usually associated with hippocampal function or the cortex) by determining how rapidly the test subjects learn to associate the US with CS. The test animals are scored by calculating the percent freezing time as determined by comparing the difference between percent freezing post-CS and the percent freezing pre-CS.

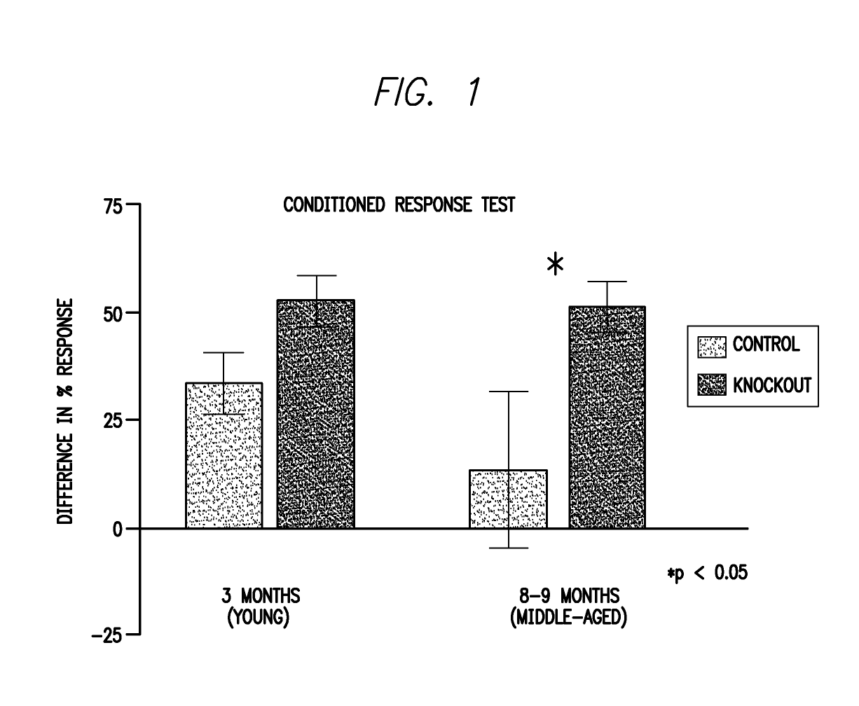

As shown in FIG. 1, both male and female animals that were homozygous for the mutation in the murine ortholog of the SLC6A7 gene displayed significantly higher freezing percentages (approximately 50 percent for an average of 16 test animals) as compared to their wildtype control counterparts (approximately 30 percent for an average of 16 control animals). These results indicate that homozygous mutant animals perform significantly better in this well established test for cognitive performance.

### 6.1.2. Water Maze

The Morris water maze used a circular pool 2 meters in diameter and 40 cm in depth. See, e.g., Morris, 1984, J. Neurosci. Methods 11:47-60, Guillou et al., 1999, J. Neurocsci. 19:6183-90. The pool was filled to a depth of 30 cm with water at a temperature of 24-26° C., made opaque by the addition of non-toxic water-based paint. The “escape” platform was about 30 cm high with a plastic disc 18 cm in diameter on top. The platform was placed about 0.5 cm below the water surface. The mouse was released into the pool facing the wall from one of 4 start positions labeled as N (North), S (South), W (West) or E (East). A videotracking system comprising the camera and the WaterMaze image software (Actimetrics, Inc.) divided the pool into 4 equal quadrants designated as SE, SW, NE, and NW. The software calculates the latency to reach platform, distance to the platform, time spent in each quadrant, swimming speed, and other parameters.

Each trial lasted until the mouse climbed onto the platform or 90 seconds had elapsed. If the mouse did not reach the platform in 90 seconds, the experimenter took it out of the water and gently placed it on the platform. At the end of each trial the mouse remained on the platform for further 20 seconds. There were 4 trials with platform per day with 8-12 min inter-trial intervals. During the inter-trial interval the mouse was kept in a clean cage under a heat lamp.

Typically one of two basic protocols were used: the first includes visible and hidden platform phases, and the second only uses a hidden platform phase; both protocols end with a 2 day reversal phase.

The visible phase generally precedes the hidden platform phase. In the visible phase, the pool was surrounded with white curtains in order to hide all external-maze cues/references. During this phase, the platform was made visible with a metal cylinder 8 cm h×3 cm, which was put on the platform. The start position was the same on each trial, while platform location was randomly changed during the trials. This phase lasted for approximately 3 days.

In the hidden platform phase, the platform was no longer marked and the curtains were removed. A variety of extra-maze cues were optionally placed around the pool. Here the start position was changed every trial, while the platform remained in the same location. This phase typically lasted about 7 days.

Probe trials were run before the training trials on day 1 and 5 of the hidden phase, and on day 1 of the visible phase, and also after the last trial on day 3 of the visible phase. During the probe trial, the platform was removed from the pool and the mouse was placed in the pool facing the wall in the quadrant opposite from the platform quadrant. The mouse swam for 60 sec and the percentage of time spent in each quadrant was recorded.

In the reversal phase, on each of 2 days, 5 trials were run. During the first trial the platform location was the same as it was in the hidden phase. In the next four trials, the platform was moved to the opposite quadrant. On the following day the platform was there on first trial and then again moved to the left or right adjoining quadrant for the last 4 trials. The start position was always kept opposite to the platform location.

When the above methods were used with SLC6A7 KO mice (n=12) and WT (n=7) controls, mice were first subjected to the visible platform task. Repeated measures (RM) and analysis of variance (ANOVA) were used to analyze genotype effect on the latency to reach platform over 11 trials.

The trial effect was F(10, 170)=8.57, p<0.001; the Genotype effect: F(1, 17)=0.65, p<0.43, interaction Genotype×Trial: F(10, 170)=0.42, p<0.93. Initially, there was no difference between WT and KO subjects, but a significant decrease in the latency over trails was observed.

When the trials progressed to the hidden platform task, RM ANOVA revealed a significant effect of the trials on the latency to reach platform: F(19, 323)=7.2, p<0.001. There was also a significant effect of genotype on same parameter: F(1, 17)=8.0, p<0.012; interaction Genotype×Trials was F(19, 323)=1.16, p<0.29. Overall, KO subjects had significantly shorter latencies to platform. No significant difference in swimming speed was detected so faster swimming did not account for the faster performance by the KO animals.

During the reversal phase, RM ANOVA was run on 4 trials with the platform switched to another quadrant on each of two days. On both days of reversal phase effect of trials was significant: Fs(3, 51)>6.4, p<0.001 indicating that both genotypes relearn well. However, there was no significant difference between them on each day of reversal: Fs(1, 17)<0.75, ps>0.39, although KO mice did tend to reach the platform faster.

During probe trials, the percent of time spent in each quadrant was compared with 25% chance for WT and KO mice by non-parametric Mann-Whitney test. The first two probe trials run before hidden phase the percent time was not different from chance in each quadrant for both genotypes. In the third probe trial run on the fifth day of hidden phase, the platform quadrant time was significantly different from chance for WT [p<0.05] and KO mice [p<0.001]; and the opposite quadrant time was significantly different for KO mice [p<0.001].

The above data indicate that KO mice learned the hidden platform task more quickly than WT animals. The data further establish that the observed difference cannot be explained by differences in visual abilities or swimming speed between genotypes.

### 6.2. Preparation of (4-Pyrimidin-2-yl-piperazin-1-yl)-[4-(4-chloromethylphenylphenyl]-methanone

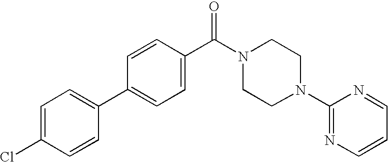

To a solution of 4′-chloro-biphenyl-4-carboxylic acid (0.1 g, 0.43 mmol) and 1-(2-pyrimidyl)-piperazine (0.07 g, 0.43 mmol) in methylene chloride (3 ml), was added EDCl (0.098 g, 0.43 mmol) and HOAt (0.07 g, 0.43 mmol) triethylamine (0.07 ml, 0.52 mmol). The mixture was stirred for 16 hours and then washed with brine. The layers were separated, and the organic phase was dried over magnesium sulfate and concentrated. The resulting oil was purified by flash chromatography, and a white solid (0.11 g) was collected. Spectral data was consistent with structure. MS (M+1)=379. HPLC (>95%). ¹H NMR (CDCl3) 8.35 (d, 2H), 7.55 (m, 8H), 6.58 (t, 1H), 3.80 (bm, 8H).

### 6.3. Preparation of (4-Pyrimidin-2-yl-piperazin-1-yl)-[6-(3-trifluoromethyl-phenyl)-pyridin-3-yl]methanone

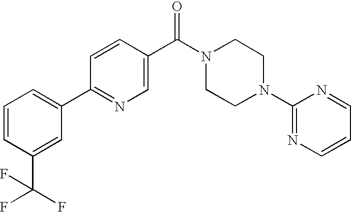

The title compound was prepared from (6-chloro-pyridin-3-yl)-(4-pyrimidin-2-yl-piperazin-1-yl)-methanone as described below.

(6-Chloro-pyridin-3-yl)-(4-pyrimidin-2-yl-piperazin-1-yl)-methanone: To a solution of chloronicotinic acid (2.51 g, 15.9 mmol) in DMF (64 ml), EDCl (4.57 g, 23.9 mmol) and HOBt (3.23 g, 23.9 mmol) were added. Hunig's base (19.3 ml, 111 mmol) was added and the reaction was allowed to stir for 5 minutes. After this induction period, piperazine (4.52 g, 19.1 mmol) was added and the reaction stirred at room temperature. After stirring for 72 hours, the reaction was diluted with ethyl acetate and water. The layers were separated, and the aqueous portion was extracted twice more with ethyl acetate. The combined organic layers were washed with water three times and once with brine, dried over MgSO₄, filtered, and concentrated. The crude product was purified by silica gel chromatography using 20-25% acetone/hexanes, yielding the product (2.05 g, 42%) as a tan solid: ¹H NMR (400 MHz, CDCl₃) δ 8.49 (d, J=1.8 Hz, 1H), 8.34 (d, J=4.7 Hz, 2H), 7.77 (dd, J=8.2, 2.4 Hz, 1H), 7.43 (d, J=8.1 Hz, 1H), 6.57 (t, J=4.8 Hz, 1H), 3.89 (bs, 6H), 3.52 (bs, 2H); m/z calcd. for C₁₄H₁₄ClN₅O: 303.08 found: (M+H)⁺ 304.1; HPLC retention time=1.822 min (gradient of solvent B-0 to 100%; wavelength 254 nm), purity=100%.

(4-Pyrimidin-2-yl-piperazin-1-yl)-[6-(3-trifluoromethyl-phenyl)-pyridin-3-yl]-ethanone: In a microwave reaction vessel, (6-chloro-pyridin-3-yl)-(4-pyrimidin-2-yl-piperazin-1-yl)-methanone (1.12 g, 3.69 mmol) was taken up in DME (15 ml). To this solution, boronic acid (1.36 g, 7.38 mmol), potassium phosphate (2.35 g, 11.1 mmol) and water (5 ml) were added. This mixture was then degassed using nitrogen, and the tetrakis triphenylphosphine palladium (0.426 g, 0.369 mmol) was added and the vessel sealed. The reaction was heated in the microwave at 160° C. for 5 minutes. After the reaction was complete, 1 N NaOH solution was added, and extraction twice with CH₂Cl₂ followed. The combined organic portions were washed with brine, dried, filtered, and concentrated. The crude product was purified by silica gel chromatography using 10-25% acetone in hexanes, yielding the final product (1.29 g, 85%) as a white solid: ¹H NMR (400 MHz, CDCl₃) δ 8.80 (d, J=1.3 Hz, 1H), 8.34 (d, J=4.8 Hz, 2H), 8.32 (s, 1H), 8.22 (d, J=7.8 Hz, 1H), 7.93 (dd, J=8.1, 2.2 Hz, 1H), 7.87 (d, J=8.1 Hz, 1H), 7.72 (d, J=7.7 Hz, 1H), 7.63 (t, J=7.8 Hz, 1H), 6.57 (t, J=4.7 Hz, 1H), 3.91 (bs, 6H), 3.60 (bs, 2H); ¹³C NMR (100 MHz, CDCl₃) δ 167.81, 161.42, 157.80, 156.93, 148.18, 139.06, 136.46, 131.52, 131.20, 130.26, 130.17, 129.39, 126.20, 126.16, 126.13, 125.36, 123.99, 123.95, 123.92, 123.88, 122.65, 120.27, 110.69; m/z calcd. for C₂₁H₁₈F₃N₅O: 413.15 found: (M+H)⁺ 414.05; HPLC retention time=3.233 min (gradient of solvent B-0 to 100%; wavelength 254 nm); purity=100%; mp=124-126° C.

### 6.4. Preparation of (4-Pyrimidin-2-yl-piperazin-1-yl)-(5-p-tolyl-pyridin-2-yl)-methanone

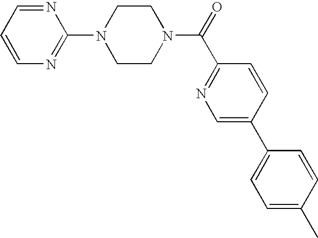

To a solution of 5-bromo-2-iodopyridine (100 mg, 0.35 mmol, Song et al., Org. Lett., 6: 4905-4907 (2004)) in THF (1 ml) was added isopropyl magnesium chloride (2 M in THF, 0.185 ml) at 0° C. After being stirred for 45 minutes, a solution of 1-pyrimidin-2-yl-1,2,3,6-tetrahydro-pyridin-4-carboxylic acid methoxy-methyl amide (61 mg, 0.245 mmol) was added. The mixture was stirred at room temperature for another 1.5 hours and quenched with addition of water (15 ml) and EtOAc (50 ml). The aqueous phase was further extracted with EtOAc (20 ml). The combined organic layers were washed with brine (10 ml), dried (MgSO₄), filtered, and concentrated under reduced pressure to furnish the crude product. This material was purified by column chromatography (3% MeOH/CH₂Cl₂) to give (5-bromo-pyridin-2-yl)-(4-pyrimidin-2-yl-piperazin-1-yl)-methanone (25 mg, 25% for two steps) as a white solid: ¹H NMR (CDCl₃, 400 MHz) δ 8.75 (m, 1H), 8.31 (d, J=6.4 Hz, 2H), 7.98 (m, 2H), 6.47 (t, J=6.4 Hz, 1H), 4.84 (m, 2H), 4.09 (m, 1H), 3.11 (m, 2H), 1.74 (m, 2H), 1.66 (m, 2H); MS calc'd. for C₁₄H₁₅BrN₅O [M+H]⁺: 349; Found: 349.

Following the general procedures for the Suzuki reactions, the title compound was obtained in 69% yield as an off-white solid: ¹H NMR (CDCl₃, 400 MHz) δ 8.92 (m, 1H), 8.33 (d, J=6.4 Hz, 2H), 8.05 (m, 1H), 7.54 (m, 1H), 6.48 (t, J=6.4 Hz, 1H), 4.85 (m, 2H), 4.22 (m, 1H), 3.12 (m, 2H), 2.44 (s, 3H), 2.02 (m, 2H), 1.75 (m, 2H); MS calc'd. for C₂₁H₂₂N₅O [M+H]⁺: 359; Found: 359.

### 6.5. Preparation of (3,4,5,6-Tetrahydro-2H-[1,2′]bipyridinyl-4-yl)-(3′-trifluoromethyl-biphenyl-4-yl)-methanone

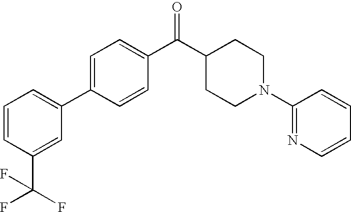

The title compound was prepared from (4-bromo-phenyl)-(3,4,5,6-tetrahydro-2H-[1,2′]bipyridinyl-4-yl)-methanone as described below.

3,4,5,6-Tetrahydro-2H-[1,2′]bipyridinyl-4-carboxylic acid methoxy-methyl-amide: In a sealed tube, Weinreb amide (0.5515 g, 1.927 mmol) was taken up in absolute ethanol (10 ml) and 2-bromopyridine (0.19 ml, 1.927 mmol) and triethylamine (0.81 ml, 5.781 mmol) were added. The tube was sealed and heated at 150° C. for at least 48 hours. The reaction was then diluted with CH₂Cl₂, washed with water and brine, dried over MgSO₄, filtered, and concentrated. The crude product was purified by silica gel chromatography using 10-20% acetone in hexanes, yielding the product (0.1375 g, 29%) as a brown oil: ¹H NMR (400 MHz, CDCl₃) δ 8.17 (dd, J=4.9, 1.2 Hz, 1H), 7.46 (m, 1H), 6.66 (d, J=8.6 Hz, 1H), 6.58 (m, 1H), 4.35 (dt, J=13.0, 2.9 Hz, 2H), 3.74 (s, 3H), 3.20 (s, 3H), 2.91 (m, 3H), 1.83 (m, 4H); m/z calcd. for C₁₃H₁₉N₃O₂: 249.15 found: (M+H)⁺ 250.05; HPLC retention time=1.533 min (wavelength 220 nm), purity=98.4%.

(4-Bromo-phenyl)-(3,4,5,6-tetrahydro-2H-[1,2′]bipyridinyl-4-yl)-methanone: A solution of 1,4-dibromobenzene (0.223 g, 0.944 mmol) in anhydrous THF (3.0 ml) was cooled to −78° C. To the cooled solution, n-butyllithium (1.6 M in hexanes, 0.47 ml, 0.746 mmol) was added dropwise, and the reaction stirred at −78° C. for 45 minutes. A solution of the 3,4,5,6-tetrahydro-2H-[1,2′]bipyridinyl-4-carboxylic acid methoxy-methyl-amide (0.124 g, 0.497 mmol) in anhydrous THF (3.0 ml) was then added dropwise to the reaction. The reaction stirred at −78° C. for 3 hours and at 0° C. until complete. The reaction was quenched at 0° C. by the addition of 1 N HCl (5 ml) and saturated NaHCO₃ (7.5 ml). The mixture was extracted with ethyl acetate, washed with brine, dried over MgSO₄, filtered, and concentrated. The crude product was purified by silica gel chromatography using 3-10% acetone in hexanes, yielding the product (0.1220 g, 71%) as a colorless oil: ¹H NMR (400 MHz, CDCl₃) δ 8.16 (dd, J=4.9, 1.2 Hz, 1H), 7.81 (m, 2H), 7.61 (m, 2H), 7.46 (m, 1H), 6.67 (d, J=8.7 Hz, 1H), 6.59 (dd, J=6.7, 5.1 Hz, 1H). 4.33 (dt, J=13.1, 3.1 Hz, 2H), 3.42 (m, 1H), 3.14 (m, 2H), 1.93 (d, J=13.2, 2.2 Hz, 2H), 1.82 (m, 2H); ¹³C NMR (100 MHz, CDCl₃) 201.20, 159.20, 147.87, 137.53, 134.57, 132.28, 129.80, 128.20, 113.09, 107.34, 45.01, 43.76, 28.01; m/z calcd. for C₁₇H₁₇BrN₂O: 344.05 found: (M+H)⁺ 347.1; HPLC retention time=3.205 min (wavelength 254 nm), purity=100%.

(3,4,5,6-Tetrahydro-2H-[1,2′]bipyridinyl-4-yl)-(3′-trifluoromethyl-biphenyl-4-yl)-methanone: In a vial, (4-bromo-phenyl)-(3,4,5,6-tetrahydro-2H-[1,2′]bipyridinyl-4-yl)-methanone (0.0634 g, 0.184 mmol) was taken up in DME (1.5 ml). To this solution, boronic acid (0.0846 g, 0.460 mmol), potassium phosphate (0.117 g, 0.551 mmol) and water (0.4 ml) were added. This mixture was then degassed using nitrogen. The tetrakis triphenylphosphine palladium (0.0213 g, 0.0184 mmol) was added, and the vial sealed. The reaction was then heated at 80° C. for 18 hours. After completion, 1 N NaOH solution was added and extraction twice with CH₂Cl₂ followed. The combined organic portions were washed with brine, dried, filtered, and concentrated. The crude product was purified by silica gel chromatography using 5-10% acetone in hexanes yielding the final product (0.042 g, 56%) as a white solid: ¹H NMR (300 MHz, CDCl₃) δ 8.19 (dd, J=4.9, 1.2 Hz, 1H), 8.08 (d, J=8.5 Hz, 2H), 7.87 (s, 1H), 7.81 (d, J=7.5 Hz, 1H), 7.72 (d, J=8.5 Hz, 2H), 7.63 (m, 2H), 7.48 (m, 1H), 6.71 (d, J=8.6 Hz, 1H), 6.62 (dd, J=6.8, 5.2 Hz, 1H), 4.34 (dt, J=13.1, 3.0 Hz, 2H), 3.54 (m, 1H), 3.06 (m, 2H), 2.01 (dd, J=13.1, 2.5 Hz, 2H), 1.92 (dd, J=11.3, 4.0 Hz, 1H), 1.84 (m, 1H); m/z calcd. for C₂₄H₂₁F₃N₂O: 410.16 found: (M+H)⁺ 411.05; HPLC retention time=3.313 min (wavelength 254 nm), purity=96.9%.

### 6.6. Preparation of (1-(Pyrimidin-2-yl)piperidin-4-yl)(4-4-trifluoromethylphenyl)-phenyl)methanone

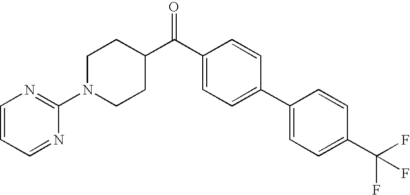

The title compound was prepared from (4-bromophenyl)(1-(pyrimidin-2-yl)piperidin-4-yl)methanone as described below.

N-methoxy-N-methylpiperidine-4-carboxamide: A mixture of N-tert-butoxycarbonyl isonipecotic acid (1.50 g, 6.54 mmol, 1 eq), 1-(3-dimethylaminopropyl)3-ethylcarbodiimide hydrochloride (1.88 g, 9.81 mmol, 1.5 eq), 1-hydroxybenzotriazole (1.33 g, 9.81 mmol, 1.5 eq), and N,N-dimethylformamide (26 ml) was treated with N,N-diisopropylethylamine (4.60 ml, 26.2 mmol, 4 eq). The resultant yellow solution was stirred at room temperature for 5 minutes, and then N,O-dimethylhydroxylamine hydrochloride (766 mg, 7.85 mmol, 1.2 eq) was added, and stirring continued for 92 hours. The reaction mixture was diluted with 100 ml of ethyl acetate and washed sequentially with 1 N aq. NaOH, 1 N aq. HCl and brine. The organic phase was dried over Na₂SO₄ and concentrated to give an oil which was used with no further purification.

This oil was dissolved in 1:2 trifluoroacetic acid/dichloromethane (9 ml), and the reaction mixture was stirred at ambient temperature for 17 hours and then concentrated. Ether (30 ml) was added and the white solid which formed was collected by filtration, washed with ether and dried to afford 1.50 g (80% yield, 2 steps) of analytically pure product: 400 MHz ¹H NMR (d₆-DMSO): 8.55 (br s, 1H), 8.25 (br s, 1H), 3.69 (s, 3H), 3.31 (m, 2H), 3.10 (s, 3H), 2.98 (m, 3H), 1.65-1.84 (m, 4H).

N-methoxy-N-methyl-1-(pyrimidin-2-yl)piperadine-4-carboxamide: A mixture of N-methoxy-N-methylpiperidine-4-carboxamide (1.50 g, 5.25 mmol, 1 eq), 2-chloropyrimidine (634 mg, 5.25 mmol, 1 eq), triethylamine (2.20 ml, 15.8 mmol, 3 eq), and ethanol (21 ml) was heated at 100° C. in a sealed tube for 19 hours. The reaction mixture was allowed to cool to room temperature and then concentrated. The residue was dissolved in dichloromethane, washed with water and brine, dried over Na₂SO₄, and concentrated. Column chromatography (silica gel, 50%→60% ethyl acetate/hexanes) gave 1.28 g (97% yield) of the product as a colorless oil: HPLC: 100% pure at 1.905 min (YMC-Pack ODS-A 4.6×33 mm column, 0%→100% solvent B over 4 min, 3 ml/min, 220 nm); LCMS (M+H)⁺=251.05; 400 MHz ¹H NMR (CDCl₃) 8.29 (d, J=4.7 Hz, 2H), 6.45 (t, J=4.7 Hz, 1H), 4.80 (m, 2H), 3.73 (s, 3H), 3.19 (s, 3H), 2.95 (m, 3H), 1.70-1.84 (m, 4H).

(4-Bromophenyl)(1-(pyrimidin-2-yl)piperidin-4-yl)methanone: A solution of 1,4-dibromobenzene (2.29 g, 9.72 mmol, 1.9 eq) in THF (20 ml) under N₂ was cooled to −78° C., and n-butyllithium (1.6 M in hexanes, 4.8 ml, 7.67 mmol, 1.5 eq) was added dropwise. The reaction mixture was stirred at −78° C. for 40 minutes, and a solution of N-methoxy-N-methyl-1-(pyrimidin-2-yl)piperadine-4-carboxamide (1.28 g, 5.11 mmol, 1 eq) in THF (5 ml) was added dropwise via a cannula. After 3 hours at −78° C., the reaction mixture was warmed to 0° C., stirred for 1 hour, and then quenched with 1 N aq. HCl (10 ml). The mixture was diluted with 150 ml of ethyl acetate, washed sequentially with saturated aq. NaHCO₃ and brine (75 ml each), and the organic phase was dried over Na₂SO₄ and concentrated. Column chromatography (silica gel, CH₂Cl₂→3.5% ethyl acetate/CH₂Cl₂) afforded 1.47 g (83% yield) of the product as a pale yellow solid: HPLC: 99% pure at 3.748 min (YMC-Pack ODS-A 4.6×33 mm column, 0%→100% solvent B over 4 min, 3 ml/min, 220 nm); LCMS (M+H)⁺=345.90; 400 MHz ¹H NMR (CDCl₃) 8.31 (d, J=4.7 Hz, 2H), 7.83 (d, J=8.5 Hz, 2H), 7.63 (d, J=8.5 Hz, 2H), 6.48 (t, J=4.7 Hz, 1H), 4.81 (m, 2H), 3.49 (m, 1H), 3.08 (m, 2H), 1.72-1.95 (m, 4H).

(1-(Pyrimidin-2-yl)piperidin-4-yl)(4-4-trifluoromethylphenyl)-phenyl methanone: A mixture of (4-bromophenyl)(1-(pyrimidin-2-yl)piperidin-4-yl)methanone (66 mg, 0.19 mmol, 1 eq), 4-trifluoromethylphenylboronic acid (91 mg, 0.47 mmol, 2.5 eq), potassium phosphate (122 mg, 0.57 mmol, 3 eq), and Pd(PPh₃)₄ (22 mg, 0.019 mmol, 0.1 eq) in 3:1 DME/water (2 ml) was heated at 80° C. under N₂ for 16 hours. The reaction mixture was cooled to room temperature, poured into 1 N NaOH, and extracted twice with dichloromethane. The combined organic layers were dried over Na₂SO₄ and concentrated. Column chromatography (silica gel, 25% ethyl acetate/hexanes) afforded 58 mg (73% yield) of the final product as a white solid: HPLC: 97% pure at 4.523 min (YMC-Pack ODS-A 4.6×33 mm column, 0%→100% solvent B over 4 min, 3 ml/min, 220 nm); LCMS (M+H)⁺=412.20; 300 MHz ¹H NMR (CDCl₃) 8.32 (d, J=4.7 Hz, 2H), 8.08 (d, J=8.4 Hz, 2H), 7.70-7.74 (m, 6H), 6.48 (t, J=4.7 Hz, 1H), 4.83 (m, 2H), 3.58 (m, 1H), 3.12 (m, 2H), 1.75-2.01 (m, 4H).

### 6.7. Preparation of (1-(Pyrimidin-2-yl)piperidin-4-yl)(4-4trifluoromethylphenyl)-phenyl)methanol

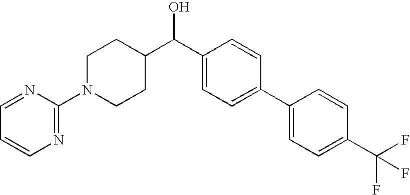

Sodium borohydride (3.0 mg, 0.080 mmol, 1.5 eq) was added to a solution of (1-(pyrimidin-2-yl)piperidin-4-yl)(4-4-trifluoromethylphenyl)phenyl)methanone (22 mg, 0.053 mmol, 1 eq) in 1:1 methanol/dichloromethane. The reaction mixture was stirred at room temperature for 1 hour and then slowly quenched with saturated aq. NaHCO₃. The biphasic mixture was extracted twice with dichloromethane, and the combined organic layers were dried over Na₂SO₄ and concentrated. Preparative TLC (500 μm silica gel, 33% ethyl acetate/hexanes) gave 17 mg (77% yield) of the product as a white solid: HPLC: 100% pure at 4.285 min (YMC-Pack ODS-A 4.6×33 mm column, 0%→100% solvent B over 4 min, 3 ml/min, 220 nm); LCMS (M+H)⁺=414.10; 300 MHz ¹H NMR (CDCl₃) 8.27 (d, J=4.7 Hz, 2H), 7.69 (s, 4H), 7.59 (d, J=8.3 Hz, 2H), 7.42 (d, J=8.2 Hz, 2H), 6.43 (t, J=4.7 Hz, 1H), 4.71-4.87 (m, 2H), 4.48 (m, 1H), 2.72-2.89 (m, 2H), 1.88-2.11 (m, 3H), 1.19-1.49 (m, 3H).

### 6.8. Preparation of Biphenyl-4-yl-(1-pyrimidin-2-yl-1,2,3,6-tetrahydro-pyridin-4-yl)-methanone

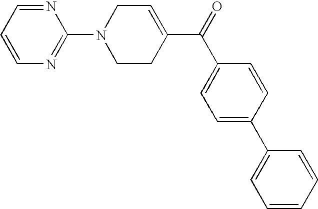

To a solution of 2-chloropyrimidine (300 mg, 2.619 mmol) in dioxane (5 ml), was added piperidin-4-one hydrochloride monohydrate (402.3 mg, 2.619 mmol) at room temperature. The mixture was heated at 80° C. overnight and concentrated under reduced pressure. The residue was treated with EtOAc (30 ml) and saturated NaHCO₃ (10 ml). After separation of the layers, the aqueous phase was extracted with EtOAc (2×10 ml). The combined organic layers were washed with brine (10 ml), dried (MgSO₄), filtered, and concentrated under reduced pressure to furnish a crude product. This material was purified by column chromatography (40% EtOAc/hexanes) to give 1-pyrimidin-2-yl-piperidin-4-one (320 mg, 53%) as an off-white solid: ¹H NMR (CDCl₃, 400 MHz) δ 8.38 (d, J=6.4 Hz, 2H), 6.61 (t, J=6.4 Hz, 9H), 4.16 (t, J=5.6 Hz, 2H), 2.53 (t, J=5.6 Hz, 2H).

To a solution of LDA (prepared from diisopropylamine (167.4 mg, 1.658 mmol) and n-BuLi (2.5 M in hexanes, 0.663 ml, 1.658 mmol) at −78° C., was added a solution of the above 1-pyrimidin-2-yl-piperidin-4-one (320 mg, 1.382 mmol). The mixture was stirred at the same temperature for 1 hour, followed by the addition of PhNTf₂ (543.1 mg, 1.52 mmol). The reaction mixture was warmed up to room temperature and stirred for 3 hours before it was quenched with the addition of saturated ammonium chloride (15 ml) and EtOAc (40 ml). After separation of the layers, the aqueous phase was extracted with EtOAc (2×10 ml). The combined organic layers were washed with brine (10 ml), dried (MgSO₄), filtered, and concentrated under reduced pressure to furnish the crude product. This material was purified by column chromatography (20% EtOAc/hexanes) to give the corresponding triflate (210.7 mg, 49%) as a white solid as long with recovered starting material (142.9 mg): ¹H NMR (CDCl₃, 400 MHz) δ 8.37 (d, J=6.4 Hz, 2H), 6.59 (t, J=6.4 Hz, 1H), 5.91 (m, 1H), 4.41 (m, 2H), 4.11 (t, J=5.6 Hz, 2H), 2.55 (m, 2H); MS calc'd for C₁₀H₁₁F₃N₃O₃S [M+H]⁺: 310; Found: 310.

To a solution of the above triflate (210.7 mg, 0.682 mmol) in methanol (10 ml), was added Pd(OAc)₂ (10.7 mg, 0.047 mmol), PPh₃ (31.3 mg, 0.119 mmol) and diisopropyl ethylamine (352.6 mg, 2.728 mmol) at room temperature. Carbon monoxide was bubbled through the solution for 4 hours before the mixture was concentrated under reduced pressure. The residue was treated with EtOAc (30 ml) and water (10 ml). The aqueous phase was further extracted with EtOAc (2×10 ml). The combined organic layers were washed with brine (10 ml), dried (MgSO₄), filtered, and concentrated under reduced pressure to furnish the crude product. This material was purified by column chromatography (30% EtOAc/hexanes) to give 1-pyrimidin-2-yl-1,2,3,6-tetrahydro-pyridin-4-carboxylic acid methyl ester (73.8 mg, 50%) as white crystals: ¹H NMR (CDCl₃, 400 MHz) δ 8.37 (d, J=6.4 Hz, 2H), 7.04 (m, 1H), 6.54 (t, J=6.4 Hz, 1H), 4.41 (m, 2H), 3.98 (t, J=5.6 Hz, 2H), 3.79 (s, 3H), 2.52 (m, 2H).

To a suspension of 1-pyrimidin-2-yl-1,2,3,6-tetrahydro-pyridin-4-carboxylic acid methyl ester (73.8 mg, 0.337 mmol) and N-methyl-O-methyl hydroxylamine hydrochloride (51.0 mg, 0.552 mmol) in THF (3 ml), was added isopropyl magnesium chloride (2.0 M in THF, 0.505 ml) at −20° C. over 15 minute-period. The mixture was stirred at −10° C. for another 30 minutes before it was quenched with the addition of saturated ammonium chloride (10 ml). The mixture was extracted with EtOAc (2×15 ml). The combined organic layers were washed with brine (15 ml), dried (MgSO₄), filtered, and concentrated under reduced pressure to furnish the crude product. This material was purified by column chromatography (4% MeOH/CH₂Cl₂) to give 1-pyrimidin-2-yl-1,2,3,6-tetrahydro-pyridin-4-carboxylic acid methoxy-methyl amide (48 mg, 58%) as white crystals: ¹H NMR (CDCl₃, 400 MHz) δ 8.35 (d, J=6.4 Hz, 2H), 6.53 (t, J=6.4 Hz, 1H), 6.43 (m, 1H), 4.35 (m, 2H), 3.99 (t, J=5.6 Hz, 2H), 3.66 (s, 3H), 3.27 (s, 3H), 2.55 (m, 2H).

To a solution of 1-pyrimidin-2-yl-1,2,3,6-tetrahydro-pyridin-4-carboxylic acid methoxy-methyl amide (48 mg, 0.196 mmol) in THF (1 ml), was added 1-biphenyl-4-yl magnesium bromide (0.5 M in THF) at 0° C. The mixture was stirred at this temperature for 1 hour and quenched with addition of water (5 ml) and EtOAc (20 ml). The aqueous phase was further extracted with EtOAc (2×8 ml). The combined organic layers were washed with brine (5 ml), dried (MgSO₄), filtered, and concentrated under reduced pressure to furnish the crude product. This material was purified by column chromatography (4% MeOH/CH₂Cl₂) to give the title compound (20 mg, 30%) as an off-white solid: ¹H NMR (CDCl₃, 400 MHz) δ 8.38 (d, J=6.4 Hz, 2H), 7.82-7.42 (m, 9H), 6.70 (m, 1H), 6.58 (t, J=6.4 Hz, 1H), 4.51 (m, 2H), 4.13 (t, J=5.6 Hz, 2H), 2.72 (m, 2H); MS calc'd for C₂₂H₂₀N₃O [M+H]⁺: 342; Found: 342.

### 6.9. Preparation of Biphenyl-4-yl-(1-pyrimidin-2-yl-1,2,3,6-tetrahydro-pyridin-4-yl)-methanol

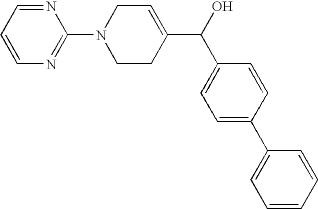

To a solution of biphenyl-4-yl-(1-pyrimidin-2-yl-1,2,3,6-tetrahydro-pyridin-4-yl)-methanone (12.2 mg, 0.0355 mmol) in methanol (0.5 ml), was added CeCl₃ heptahydate (13.2 mg, 0.0355 mmol) and sodium borohydride (1.5 mg, 0.0355 mmol) at room temperature. The mixture was stirred for 1 hour and diluted with EtOAc (10 ml). The mixture was washed with water (5 ml), brine (5 ml), dried (MgSO₄), filtered, and concentrated under reduced pressure to furnish the crude product. This material was purified by column chromatography (6% MeOH/CH₂Cl₂) to give the title compound (12 mg, 98%) as a white gel: ¹H NMR (CDCl₃, 400 MHz) δ 8.36 (d, J=6.4 Hz, 2H), 7.62-7.37 (m, 9H), 6.46 (t, J=6.4 Hz, 1H), 6.02 (m, 1H), 5.24 (m, 1H), 4.31 (m, 2H), 3.96 (m, 1H), 3.83 (m, 1H), 2.14 (m, 2H); MS calc'd for C₂₂H₂₂N₃O [M+H]⁺: 344; Found: 344.

### 6.10. Preparation of 2-[4-(Biphenyl-4-yloxy)-piperidin-1-yl]-pyrimidine

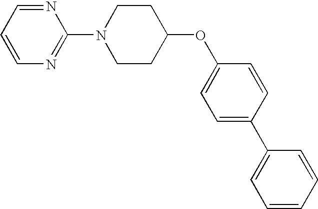

To a solution of 1-pyrimidin-2-yl-piperidin-4-one (50 mg, 0.282 mmol) in methanol (0.8 ml), was added sodium borohydride (12.0 mg, 0.282 mmol) at room temperature. After being stirred for 10 minutes, the mixture was treated with EtOAc (10 ml) and water (3 ml). The organic layer was washed with brine (2 ml), dried (MgSO₄), filtered, and concentrated under reduced pressure to furnish the crude product. This material was purified by column chromatography (20% EtOAc/hexanes) to give the corresponding alcohol (51 mg, 100%) as a white solid.

To a mixture of the above alcohol (50 mg, 0.279 mmol), PPh₃ (109.6 mg, 0.418 mmol) and biphenyl-4-ol (57.0 mg, 0.335 mmol) in THF (3 ml), was added DEAD (40% in toluene, 0.152 ml, 0.335 mmol) at 0° C. After being stirred overnight, the mixture was treated with EtOAc (15 ml) and water (5 ml). The aqueous phase was extracted with EtOAc (2×5 ml). The combined organic layers were washed with brine (5 ml), dried (MgSO₄), filtered, and concentrated under reduced pressure to furnish the crude product. This material was purified by column chromatography (15% EtOAc/hexanes) to give the title compound (81 mg, 88%) as white crystals: ¹H NMR (CDCl₃, 400 MHz) δ 8.38 (d, J=6.4 Hz, 2H), 7.59-7.04 (m, 9H), 6.61 (t, J=6.4 Hz, 1H), 4.62 (m, 1H), 4.21 (m, 2H), 3.68 (m, 2H), 2.14 (m, 2H), 1.83 (m, 2H); MS calc'd for C₂₁H₂₂N₃O [M+H]⁺: 332; Found: 332.

### 6.11. Preparation of (3′-Chloro-biphenyl-4-yl)-(4-thiazol-2-yl-piperazin-1-yl)-methanone

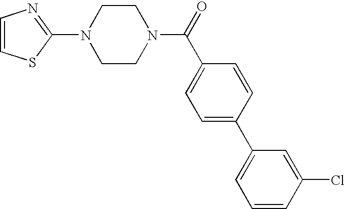

To a solution of 1-(thiazol-2-yl)piperazine (ca. 0.915 mmol, prepared from 150 mg 2-bromothiazole according to the methods described in Astles et al., J. Med. Chem., 39: 1423-1432 (1996)), 3′-chloro-biphenyl-4-carboxylic acid (212.9 mg, 0.915 mmol) in CH₂Cl₂ (4 ml), was added EDC (209.7 mg, 1.098 mmol) and HOBt (148.2 mg, 1.098 mmol). After being stirred overnight, the mixture was treated with EtOAc (50 ml) and water (15 ml). The organic phase was washed with brine (5 ml), dried (MgSO₄), filtered, and concentrated under reduced pressure to furnish the crude product. This material was purified by column chromatography (20% acetone/hexanes) to give the title compound (225 mg, 64% for two steps) as a white solid: ¹H NMR (CDCl₃, 400 MHz) δ 7.64-7.23 (m, 9H), 6.65 (t, J=3.6 Hz, 1H), 4.92 (m, br, 2H), 3.57 (m, br, 6H), 3.68 (m, 2H), 2.14 (m, 2H), 1.83 (m, 2H); MS calc'd for C₂₀H₁₉ClN₃OS [M+H]⁺: 384; Found: 384.

### 6.12. Preparation of 4-(4′-Chloro-biphenyl-4-yl)-1-pyrimidin-2-yl-piperidin-4-ol

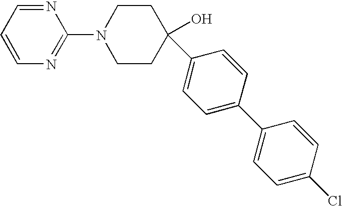

To a solution of 1,4-dibromobenzene (213.3 mg, 0.904 mmol) in THF (4 ml), was added n-BuLi (2.5 M in hexanes, 0.362 ml, 0.904 mmol) at −78° C. After being stirred for 30 minutes at the same temperature, a solution of 1-pyrimidin-2-yl-piperidin-4-one (80 mg, 0.452 mmol) in THF (3 ml) was added. The mixture was allowed to warm to room temperature and stirred for 1 hour. The reaction was quenched with addition of water (10 ml) and EtOAc (50 ml). The organic layer was washed with brine (5 ml), dried (MgSO₄), filtered, and concentrated under reduced pressure to furnish the crude product. This material was purified by column chromatography (40% EtOAc/hexanes) to give 4-(4-Bromo-phenyl)-1-pyrimidin-2-yl-piperidin-4-ol as a colorless oil (140 mg, 93%): ¹H NMR (CDCl₃, 400 MHz) δ 8.33 (d, J=6.4 Hz, 2H), 7.47 (d, J=12.0 Hz, 2H), 7.41 (d, J=12.0 Hz, 2H), 6.49 (t, J=6.4 Hz, 1H), 4.72 (m, 2H), 3.40 (m, 2H), 2.05 (m, 2H), 1.78 (m, 2H); MS calc'd for C₁₅H₁₇BrN₃O [M+H]⁺: 335; Found: 335.

Following the general procedures for the Suzuki reactions, the title compound was prepared in 61% yield as a colorless glass: ¹H NMR (CDCl₃, 400 MHz) δ 8.35 (d, J=6.4 Hz, 2H), 7.59-7.37 (m, 8H), 6.50 (t, J=6.4 Hz, 1H), 4.73 (m, 2H), 3.46 (t, J=12.4 Hz, 2H), 2.15 (m, 2H), 1.88 (m, 2H); MS calc'd for C₂₁H₂₁ClN₃O [M+H]⁺: 366; Found: 366.

### 6.13. Preparation of Biphenyl-4-yl-(1-pyrimidin-2-yl-azetidin-3-yl)-methanone

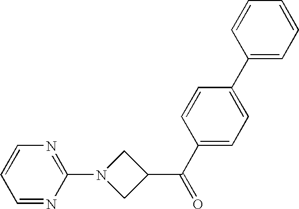

To a stirred solution of 3-azetidine carboxylic acid methyl ester hydrochloride (150 mg, 0.99 mmol) and 2-chloropyrimidine (113.4 mg, 0.99 mmol) in methanol, was added TEA (200 mg, 1.98 mmol) at room temperature. The mixture was stirred at 50° C. for 5 hours and concentrated under reduced pressure. The residue was suspended in EtOAc (50 ml) and washed with water (15 ml), brine (5 ml), dried (MgSO₄), filtered, and concentrated under reduced pressure to furnish the crude product. This material was purified by column chromatography (40% EtOAc/hexanes) to give 1-pyrimidin-2-yl-azetidine-3-carboxylic acid methyl ester as a light yellow solid (137.3 mg, 72%): ¹H NMR (CDCl₃, 400 MHz) δ 8.37 (d, J=6.4 Hz, 2H), 6.58 (t, J=6.4 Hz, 1H), 4.30 (m, 4H), 3.77 (s, 3H), 3.56 (m, 1H).

To a suspension of the above ester (137.3 mg, 0.711 mmol) and N-methyl-O-methyl hydroxylamine hydrochloride (127.6 mg, 1.103 mmol) in THF (5 ml), was added iso-propyl magnesium chloride (2.0 M in THF, 1.067 ml, 2.133 mmol) at −20° C. during 15 minutes. The mixture was stirred at −10° C. for another 30 minutes before it was quenched with the addition of saturated ammonium chloride (10 ml). The mixture was extracted with EtOAc (2×15 ml). The combined organic layers were washed with brine (10 ml), dried (MgSO₄), filtered, and concentrated under reduced pressure to furnish the crude product. This material was purified by column chromatography (4% MeOH/CH₂Cl₂) to give 1-pyrimidin-2-yl-azetidine-3-carboxylic acid methoxy-methyl-amide (385.9 mg, 98%) as a white solid: ¹H NMR (CDCl₃, 400 MHz) δ 8.32 (d, J=6.4 Hz, 2H), 6.55 (t, J=6.4 Hz, 1H), 4.34 (m, 4H), 3.88 (m, 1H), 3.70 (s, 3H), 3.23 (s, 3H).

To a solution of the above amide (50 mg, 0.225 mmol) in THF (1 ml), was added 4-biphenyl magnesium chloride (0.5 M in THF, 0.9 ml, 0.45 mmol) at −78° C. The mixture was slowly warmed up to room temperature and stirred for 2 hours before quenched with addition of water (10 ml) and EtOAc (30 ml). The organic layer was separated and washed with brine (5 ml), dried (MgSO₄), filtered, and concentrated under reduced pressure to furnish the crude product. This material was purified by column chromatography (3% MeOH/CH₂Cl₂) to furnish the title compound (21 mg, 30%) as white crystals: ¹H NMR (CDCl₃, 400 MHz) δ 8.33 (d, J=6.4 Hz, 2H), 7.98-7.43 (m, 9H), 6.58 (t, J=6.4 Hz, 1H), 4.45 (m, 4H), 4.38 (m, 1H); MS Calc'd for C₂₀H₁₈N₃O [M+H]⁺: 316; Found: 316.

### 6.14. Preparation of (3′-Chloro-biphenyl-4-yl)-(1-pyrimidin-2-yl-pyrrolidin-3-yl)-methanone

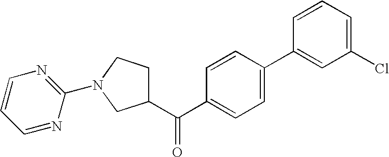

To a solution of N-Boc-β-proline (400 mg, 1.858 mmol), EDC (425.9 mg, 2.23 mmol) and HOBt (326.1 mg, 2.415 mmol) in methylene chloride (8 ml), was added N-methyl-O-methyl hydroxylamine hydrochloride (217.5 mg, 2.23 mmol) and TEA (281.5 mg, 2.787 mmol) at 0° C. After stirring overnight, the mixture was treated with EtOAc (80 ml) and water (15 ml). The organic phase was washed with brine (15 ml), dried (MgSO₄), filtered, and concentrated under reduced pressure to furnish the crude product.

To a solution of the above crude ester in methylene chloride (4 ml), was added dropwise TFA (4 ml) at room temperature. The mixture was stirred for 40 minutes and concentrated under reduced pressure to generate the crude product as the TFA salt.

To a mixture of the above product and 2-chloropyrimidine (212.8 mg, 1.858 mmol) in dioxane (7 ml) was added TEA (563 mg, 5.574 mmol). The mixture was heated at 80° C. for 4 hours, and was concentrated under reduced pressure. The residue was treated with water (20 ml) and EtOAc (60 ml). After separation of the layers, the aqueous phase was further extracted with EtOAc (20 ml). The combined organic layers were washed with brine (10 ml), dried (MgSO₄), filtered, and concentrated under reduced pressure to furnish the crude product. This material was purified by column chromatography (40% acetone/hexanes) to furnish 1-pyrimidin-2-yl-pyrrolidine-3-carboxylic acid methoxy-methyl-amide (203.8 mg, 47% for three steps) as an off-white solid: ¹H NMR (CDCl₃, 400 MHz) δ 8.34 (d, J=6.4 Hz, 2H), 6.50 (t, J=6.4 Hz, 1H), 3.94 (m, 1H), 3.82 (m, 1H), 3.75 (s, 3H), 3.70 (m, 1H), 3.65 (m, 1H), 3.63 (m, 1H), 3.23 (s, 3H), 2.33 (m, 3H), 2.23 (m, 1H).

To a solution of 1,4-dibromobenzene (407.5 mg, 1.727 mmol) in THF (6 ml) was added n-BuLi (2.5 M in hexanes, 0.691 ml, 1.727 mmol) at −78° C. The mixture was stirred at the temperature for 30 minutes before the addition of a solution of the above amide (203.8 mg, 0.8636 mmol) in THF (4 ml). After stirring at −78° C. for 30 minutes, the mixture was warmed to room temperature for 1 hour. EtOAc (40 ml) and water (15 ml) was added to the reaction, followed by separation of the layers. The aqueous phase was extracted with EtOAc (15 ml). The combined organic layers were washed with brine (10 ml), dried (MgSO₄), filtered, and concentrated under reduced pressure to furnish the crude product. This material was purified by column chromatography (40% acetone/hexanes) to furnish (4-bromo-phenyl)-(1-pyrimidin-2-yl-pyrrolidin-3-yl)-methanone (182.2 mg, 64%) as an off-white solid: ¹H NMR (CDCl₃, 400 MHz) δ 8.32 (d, J=6.4 Hz, 2H), 7.87 (d, J=12.0 Hz, 2H), 7.63 (d, J=12.0 Hz, 2H), 6.51 (t, J=6.4 Hz, 1H), 4.07 (m, 1H), 3.98 (m, 1H), 3.86 (m, 1H), 3.74 (m, 2H), 2.38 (m, 2H).

Following the general procedures for the Suzuki reactions, the title compound was prepared in 63% as a pale yellow solid: ¹H NMR (CDCl₃, 400 MHz) δ 8.38 (d, J=6.4 Hz, 2H), 8.11-7.42 (m, 8H), 6.53 (t, J=6.4 Hz, 1H), 4.19 (m, 1H), 4.04 (m, 1H), 3.84 (m, 1H), 3.77 (m, 2H), 2.42 (m, 1H), 2.38 (m, 1H); MS Calc'd for C₂₁H₁₉ClN₃O [M+H]⁺: 364; Found: 364.

### 6.15. Preparation of (4-Pyrimidin-2-yl-homopiperazin-1-yl)-[4-(3-trifluoromethylphenyl-phenyl]-methanone

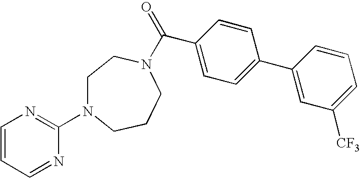

The title compound was prepared from 1-(2-pyrimidyl)-homopiperazine as described below.

1-(2-Pyrimidyl)-homopiperazine: To a solution of homopiperazine (3.5 g, 35 mmol) in ethanol (100 ml) at 40° C., was added portionwise 2-chloropyrimidine (2.0 g, 17.5 mmol). The mixture was stirred for 1 hour then concentrated in vacuo. The residue was dissolved in methylene chloride (75 ml) and washed with a saturated solution of sodium bicarbonate and brine. Layers were separated, and the organic layer was dried over magnesium sulfate and concentrated. The resulting residue was purified by flash chromatography and a semi-solid (1.0 g) was collected and used as is.

(4-Pyrimidin-2-yl-homopiperazin-1-yl)-[4-(3-trifluoromethylphenylphenyl]-methanone: To a solution of 3′-trifluoromethyl-biphenyl-4-carboxylic acid (0.38 g, 1.41 mmol) and 1-(2-pyrimidyl)-homopiperazine (0.25 g, 1.41 mmol) in methylene chloride (20 ml), was added EDCl (0.27 g, 1.41 mmol) and HOAt (0.19 g, 1.41 mmol) triethylamine (0.20 ml, 1.41 mmol). The mixture was stirred for 16 hours and then washed with brine. The layers were separated, and the organic phase was dried over magnesium sulfate and concentrated. The resulting oil was purified by flash chromatography and a clear oil was collected. The oil was dissolved in a minimal amount of t-butylmethylether, and crystals were formed collected (0.20 g). Spectral data was consistent with structure. MS (M+1)=427. HPLC (>95%). ¹H NMR (CDCl3) 8.35 (m, 2H), 7.55 (m, 8H), 6.58 (t, 1H), 3.87 (bm, 8H), 1.92 (m, 2H).

### 6.16. Preparation of (3′-Chloro-biphenyl-4-yl)-(5-pyrimidin-2-yl-hexahydro-pyrrolo[3,4-c]pyrrol-2-yl)-methanone

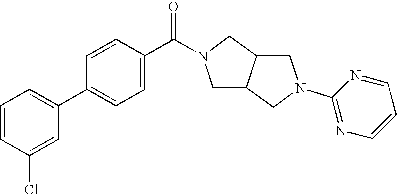

The title compound was prepared from 5-pyrimidin-2-yl-hexahydro-pyrrolo[3,4-c]pyrrole-2-carboxylic acid tert-butyl ester as described below.

5-Pyrimidin-2-yl-hexahydro-pyrrolo[3,4-c]pyrrole-2-carboxylic acid tert-butyl ester: A solution of hexahydro-pyrrolo[3,4-c]pyrrole-2-carboxylic acid tert-butyl ester (1.0 g, 4.7 mmol), 2-chloropyrimidine (0.54 g, 4.7 mmol), triethylamine (2 ml, 14 mmol) and ethyl alcohol (25 ml) was maintained at reflux for 4 hours. The solution was then cooled to room temperature and concentrated to afford a solid residue that was dissolved in dichloromethane (CH₂Cl₂), which was washed sequentially with sat. aq. sodium bicarbonate and brine, dried (Na₂SO₄), filtered, and concentrated to afford 0.82 g (60%) of the product as an orange solid: ¹H NMR (400 MHz, CDCl₃): δ 8.34 (d, J=4.8 Hz, 2H), 6.53 (t, J=4.8 Hz, 1H), 3.86-3.79 (m, 2H), 3.72-3.62 (m, 2H), 3.57-3.50 (m, 2H), 3.41-3.33 (m, 1H), 3.33-3.26 (m, 1H), 3.05-2.96 (m, 2H), 1.47 (s, 9H); LRMS m/z 291 (M+H)⁺.

(3′-Chloro-biphenyl-4-yl)-(5-pyrimidin-2-yl-hexahydro-pyrrolo[3,4-c]pyrrol-2-yl)-methanone: A solution of 5-pyrimidin-2-yl-hexahydro-pyrrolo[3,4-c]pyrrole-2-carboxylic acid tert-butyl ester (0.70 g, 2.4 mmol) and CH₂Cl₂ (20 ml) was treated with trifluoroacetic acid (TFA, 10 ml) and maintained at room temperature for 3 hours. The resulting solution was concentrated, and the residue was dissolved in CH₂Cl₂ (5 ml) and added to a solution of 3′-chloro-biphenyl-4-yl-carboxylic acid (0.62 g, 2.6 mmol), O-(7-Azabenzotriazol-1-yl)-N,N,N′,N′-tetramethyluroniumhexafluorophosphate (HATU, 1.0 g, 2.6 mmol), diisopropylethylamine (1.5 ml, 8 mmol), and CH₂Cl₂ (20 ml). The resulting solution was maintained at room temperature for 2 hours, diluted with EtOAc, washed with sat. aq. NaHCO₃ and brine, dried (MgSO₄), filtered, and concentrated. The solid residue was recrystallized from methyl alcohol to afford the final product as white needles: ¹H NMR (CD₃OD): δ 8.32 (d, J=4.8 Hz, 2H), 7.71 (d, J=8.5 Hz, 2H), 7.67 (s, 1H), 7.63 (d, J=8.5 Hz, 2H), 7.60-7.50 (m, 1H), 7.45 (t, J=7.9 Hz, 1H), 7.40-7.37 (m, 1H), 6.63 (t, J=4.8 Hz, 1H), 3.96 (dd, J=7.8, 12.8 Hz, 1H), 3.86 (ddd, J=3.0, 7.2, 10.6 Hz, 2H), 3.76 (dd, J=7.5, 11.6 Hz, 1H), 3.65-3.58 (m, 2H), 3.51 (dd, J=5.1, 11.3 Hz, 1H), 3.43 (dd, J=4.7, 11.7 Hz, 1H), 3.21-3.07 (m, 2H). ¹³C NMR (100 MHz, CD₃OD): δ 171.8, 161.4, 159.1, 143.5, 142.9, 137.0, 136.0, 131.6, 129.0, 128.2, 128.1, 126.6, 110.9, 54.5, 51.9, 51.7, 51.1, 43.9, 42.0; LRMS m/z 405 (M+H)⁺; Anal. calcd for C₂₃H₂₁ClN₄O: C, 68.23; H, 5.23; N, 13.84. Found: C, 68.01; H, 5.23; N, 13.60.

### 6.17. Preparation of (2′,4′-Difluoro-biphenyl-4-yl)-(8-pyrimidin-2-yl-8-aza-bicyclo[3.2.1]oct-3-yl)-methanone

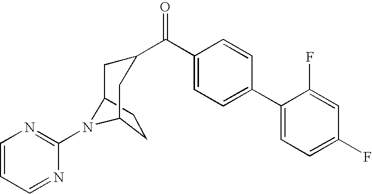

The title compound was prepared as follows.

8-Pyrimidin-2-yl-8-aza-bicyclo[3.2.1]octan-3-one: A solution of 8-aza-bicyclo [3.2.1]octan-3-one hydrochloric acid (5.0 g, 30.9 mmol), 2-chloro-pyrimidine (4.95 g, 43.2 mmol), NaHCO₃ (7.78 g, 92.7 mmol) and isopropanol (200 ml) was maintained at reflux over weekend. The resulting reaction mixture was concentrated and purified by ISCO to afford 8-pyrimidin-2-yl-8-aza-bicyclo[3.2.1]octan-3-one (4.0 g, 52.9%) as a white solid: MS (M+1)=204. ¹H NMR (MeOH) 8.36 (d, J=12 Hz 2H), 6.75 (m, 1H), 4.97 (m, 2H), 2.75 (d, J=12 Hz, 1H), 2.71 (d, J=12 Hz, 1H), 2.32 (d, J=50 Hz, 2H), 2.22 (m, 2H), 1.87 (m, 2H).

3-[(4-Bromo-phenyl)-methoxy-methylene]-8-pyrimidin-2-yl-8-aza-bicyclo[3.2.1]octane: To a solution of [(4-bromo-phenyl)-methoxy-methyl]-phosphonic acid diethyl ester (4.58 g, 13.5 mmol) in 1,2-dimethoxy-ethane (60 ml), was added NaH (540 mg, 13.5 mmol, 60% in mineral oil) in one portion. The mixture was stirred at 50° C. for 1.5 hrs before it was added by 8-pyrimidin-2-yl-8-aza-bicyclo[3.2.1]octan-3-one (2.0 g, 9.85 mmol) in 1,2-dimethoxy-ethane (5 ml). The mixture was stirred at 50° C. over the weekend. The resulting mixture was concentrated down and purified by ISCO to afford 3-[(4-bromo-phenyl)-methoxy-methylene]-8-pyrimidin-2-yl-8-aza-bicyclo[3.2.1]octane (600 mg, 30%). The white solid product was used as it was. MS (M+1)=386.

(4-Bromo-phenyl)-(8-pyrimidin-2-yl-8-aza-bicyclo[3.2.1]oct-3-yl)-methanone: A solution of 3-[(4-bromo-phenyl)-methoxy-methylene]-8-pyrimidin-2-yl-8-aza-bicyclo[3.2.1]octane (2.44 g, 6.32 mmol), aqueous HCl (10.5 ml, 6N) and THF (50 ml) was stirred at room temperature overnight. The mixture was added by saturated aq. NaHCO₃ until bubbling was gone. The mixture was diluted with ethyl acetate and the organic phase was dried over MgSO₄ and concentrated. ISCO was used to do purification and (4-bromo-phenyl)-(8-pyrimidin-2-yl-8-aza-bicyclo[3.2.1]oct-3-yl)-methanone was obtained as white solid (2.16 g, 92%). MS (M+1)=374. 1H NMR (CDCl₃) 8.33 (d, J=12 Hz 2H), 7.84 (d, J=13 Hz 2H), 7.62 (d, J=13 Hz 2H), 6.51 (t, J=12 Hz 1H), 4.87 (m, 2H), 3.90 (m, 1H), 2.23 (m, 2H), 2.08 (m, 2H), 1.97 (m, 2H), 1.72 (m, 2H).

(2′,4′-Difluoro-biphenyl-4-yl)-(8-pyrimidin-2-yl-8-aza-bicyclo[3.2.1]oct-3-yl)-methanone: A solution of (4-bromo-phenyl)-(8-pyrimidin-2-yl-8-aza-bicyclo[3.2.1]oct-3-yl)-methanone (250 mg, 0.92 mmol), 2,4-di-fluoro-phenylboronic acid (290 mg, 1.84 mmol), [1,1′-bis(diphenylphosphino)ferrocene]dichloropalladium(II), complex with dichloromethane (1:1) (67 mg, 0.092 mmol), K₃PO₄ (390 mg, 1.84 mmol), 2-dimethoxy-ethane (5 ml) and water (1.6 ml) was stirred at 80° C. for one hour. The reaction mixture was diluted with ethyl acetate and 1N NaOH solution. The organic layer was dried by MgSO₄ and concentrated. ISCO was used for purification, and the product was obtained as a white solid (2′,4′-difluoro-biphenyl-4-yl)-(8-pyrimidin-2-yl-8-aza-bicyclo[3.2.1]oct-3-yl)-methanone (69.6 mg, 19%). MS (M+1)=406. 1H NMR (CDCl₃) 8.34 (d, J=12 Hz 2H), 8.00 (d, J=21 Hz 2H), 7.62 (dd, J=19 Hz, 4 Hz, 2H), 7.45 (m, 1H), 6.99 (m, 2H), 6.52 (t, J=12 Hz, 1H), 4.90 (m, 2H), 4.00 (m, 1H), 2.25 (m, 2H), 2.12 (m, 2H), 2.02 (m, 2H), 1.78 (m, 2H).

### 6.18. Preparation of (3-(Pyrimidin-2-yl)-3,8-diazabicyclo[3.2.1]octan-8-yl)(3′-(trifluoromethyl)biphenyl-4-yl)methanone

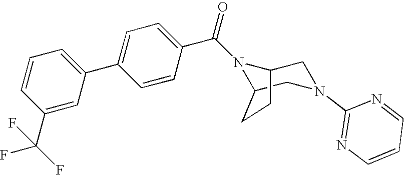

The title compound was prepared as follows.

3-Pyrimidin-2-yl-3,8-diaza-bicyclo[3.2.1]octane-8-carboxylic acid tert-butyl ester: A solution of 3,8-diaza-bicyclo[3,2,1]octane-8-carboxyli acid tert-butyl ester (50 mg, 0.24 mmol), 2-chloropyrimidine (27 mg, 0.24 mmol), triethylamine (0.1 ml, 0.72 mmol) and THF (2.5 ml) was heated at 180° C. for 10 minutes. The solution was concentrated to afford a solid residue that was dissolved in dichloromethane, which was washed sequentially with sat. aq. sodium bicarbonate and brine, dried (Na₂SO₄), filtered, and concentrated to afford 50 mg (71%) of the product as a brown solid: ¹H NMR (400 MHz, CDCl₃): δ 8.30 (d, J=12.0 Hz, 2H), 6.52 (t, J=12.0 Hz, 1H), 4.38-4.29 (m, 4H), 3.13 (sb, 2H), 2.42 (m, 2H), 1.69 (q, J=18.0 Hz, 2H), 1.49 (s, 9H); MS (M+1)=291.

(3′-tert-Butyl-biphenyl-4-yl)-(3-pyrimidin-2-yl-3,8-diaza-bicyclo[3.2.1]oct-8-yl)-methanone: A solution of 3-pyrimidin-2-yl-3,8-diaza-bicyclo[3.2.1]octane-8-carboxylic acid tert-butyl ester (64 mg, 0.22 mmol) in HCl/dioxane was stirred for 5 hours at room temperature. The resulting solution was concentrated, and the residue was dissolved in CH₂Cl₂ (5 ml) and added to a solution of 3′-trifluoromethyl-biphenyl-4-carboxylic acid (117 mg, 0.44 mmol), EDC (85 mg, 0.44 mmol), HOBt (60 mg, 0.44 mol) and TEA (0.1 ml, 0.71 mmol). After stirring overnight, the mixture was treated with EtOAc (50 ml) and water (15 ml). The organic phase was washed with brine (5 ml), dried (MgSO₄), filtered, and concentrated under reduced pressure to furnish the crude product. This material was purified by column chromatography (30% EtOAc/hexanes) to give the title compound 14.2 mg, (32%) as a white solid: ¹H NMR (DMSO): δ 8.35 (d, J=12 Hz, 2H), 7.79-7.76 (m, 3H), 7.68 (dt, J=20, 3 Hz, 1H), 7.63 (d, J=21 Hz, 2H), 7.52-7.43 (m, 2H), 6.62 (t, J=12 Hz, 1H), 4.77 (bs, 1H), 4.41 (d, J=64 Hz, 2H), 4.17 (bs, 1H), 3.14 (bs, 2H), 2.48 (qt, J=5 Hz, 1H), 1.86 (t, J=9 Hz, 2H), 1.60 (d, J=24 Hz, 1H); MS (M+1)=439.

### 6.19. Preparation of (2-Amino-4,6-dichloro-pyrimidin-5-yl)-acetaldehyde

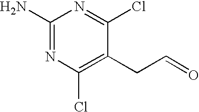

5-Allyl-2-amino-pyrimidine-4,6-diol (3): Under a nitrogen atmosphere, NaOEt was prepared by dissolving sodium metal (4.30 g, 187 mmol) into 100 ml of EtOH. At 0° C. guanidine (1) (4.80 g, 50.2 mmol) was added and the solution was stirred for 10 minutes. Diethyl allyl malonate (2) (10 ml, 50.4 mmol) was added dropwise after which the mixture was allowed to warm to room temperature. After stirring for 65 hours the reaction was quenched with 20 ml of concentrated HCl. The precipitate was filtered and washed with water and ethanol yielding pyrimidine 3 (4.29 g, 51%) as a white solid: ¹H NMR (300 MHz, (CD₃)₂SO) δ 10.32 (s, 2H) 6.37 (s, 2H), 5.81-5.68 (m, 1H), 4.91-4.78 (m, 2H), and 2.85 (d, J=6.0 Hz, 2H); m/z calcd. for C7H9N3O2: 167.17 found: (M+H)⁺ 168.10; HPLC retention time=0.677 min (gradient of solvent B-0 to 100%; wavelength 220 nM).

5-Allyl-4,6-dichloro-pyrimidin-2-ylamine (4): Under a nitrogen atmosphere, pyrimidine 3 (1.027 g, 6.15 mmol) was added to 10 ml of POCl₃. The mixture was refluxed at 110° C. After stirring for 30 min the POCl₃ was removed with the rotary evaporator. The crude mixture was very slowly quenched with 15 ml of hot distilled water. The aqueous mixture was extracted twice with CH₂Cl₂. The organic layers were combined, washed with a 1:1 mixture of saturated NaHCO_{3(aq)}/brine, dried over MgSO₄ and concentrated to yield pyrimidine 4 (320 mg, 26%) as a beige solid: ¹H NMR (300 MHz, (CDCl₃) δ 5.93-5.80 (m, 1H), 5.20-5.06 (m, 2H), and 3.52-3.49 (m, 2H); m/z calcd. for C7H7Cl2N3: 204.06 found: 204.00; HPLC retention time=3.631 min (gradient of solvent B-0 to 100%; wavelength 220 nM).

3-(2-Amino-4,6-dichloro-pyrimidin-5-yl)-propane-1,2-diol (5): To a stirring solution of pyrimidine 4 (320 mg, 1.58 mmol) in 15 ml of THF and 3 ml of water was added NMO (370 mg, 3.15 mmol) and then a few crystals of osmium tetroxide. The reaction flask was covered to block exposure to light and the mixture was stirred at room temperature. After 12 h of stirring 10 ml of an aqueous solution of NaHSO₃ (500 mg) was added to the mixture and allowed to stir for a few minutes. The mixture was filtered and the precipitate was washed with water and then triturated with Et₂O to yield some diol 5 as a white solid. The filtrate was extracted three times with EtOAc. The organic phases were combined, washed with brine, dried over MgSO₄ and concentrated in vacuo to yield more diol 5 as a white solid that was combined with the precipitated solid (329 mg, 88%): ¹H NMR (300 MHz, (CD₃)₂SO) δ 7.29 (s, 2H), 4.70 (d, J=5.1 Hz, 1H), 4.62 (t, J=5.9 Hz, 1H), 3.75-3.65 (m, 1H), 2.77-2.60 (m, 2H); m/z calcd. for C7H9Cl2N3O2: 238.07 found: 238.10; HPLC retention time=1.703 min (gradient of solvent B-0 to 100%; wavelength 220 nM).

(2-Amino-4,6-dichloro-pyrimidin-5-yl)-acetaldehyde (6): Under a nitrogen atmosphere, to a stirring suspension of diol 5 (329 mg, 1.39 mmol) in 10 ml of THF and 5 ml of methanol at 0° C. was added lead acetate (700 mg, 1.58 mmol). The mixture was stirred at 0° C. for 1 h and then diluted with EtOAc. The mixture was filtered through Celite. The filtrate was washed three times with a mixture of 1:1 saturated NaHCO_{3(aq)}/brine, dried over MgSO₄ and then concentrated to give aldehyde 6 (253 mg, 88%) as a white solid: m/z calcd. for C7H9Cl2N3O2: 206.03 found: 206.00; HPLC retention time=2.048 min (gradient of solvent B-0 to 100%; wavelength 220 nM).

### 6.20. Preparation of N-(7-tert-Butyl-5-iodo-7H-pyrrolo[2,3-d]pyrimidin-2-yl)-4-methyl-benzamide

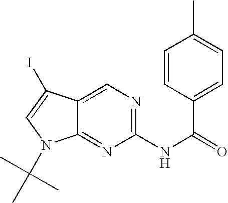

7-tert-Butyl-4-chloro-7H-pyrrolo[2,3-d]pyrimidin-2-ylamine (7): In a sealed pressure vessel aldehyde 6 (253 mg, 1.23 mmol) was suspended in 15 ml of n-butanol. To this mixture was added tert-butyl amine (0.30 ml, 2.78 mmol). After stirring for 5 min at room temperature, triethylamine (0.80 ml, 5.56 mmol) was added and the mixture was stirred in the sealed tube at 115° C. After 14 h the n-butanol was removed with the rotary evaporator. The crude product was purified by silica gel column chromatography (100% DCM) to give chloropyrrolopyrimidine 7 (170 mg, 62%): ¹H NMR (300 MHz, (CDCl₃) δ 7.05 (d, J=3.6 Hz, 1H), 6.35 (d, J=3.9 Hz, 1H), 4.90 (bs, 2H); m/z calcd. for C10H13ClN4: 224.69 found: 225.10; HPLC retention time=3.848 min (gradient of solvent B-0 to 100%; wavelength 220 nM).

7-tert-Butyl-7H-pyrrolo[2,3-d]pyrimidin-2-ylamine (8): Chloropyrrolopyrimidine 7 (308 mg, 1.38 mmol) was dissolved in 25 ml of methanol. To this was added 3 ml concentrated ammonia and a catalytic amount of palladium on carbon. The mixture was stirred under a hydrogen atmosphere at room temperature. After stirring for 2.5 h the mixture was filtered through Celite and the filtrate was concentrated. The crude product was passed through a plug of silica gel to yield pyrrolopyrimidine 8 (240 mg, 92%) as a yellow solid: m/z calcd. for C10H14N4: 190.25 found: 191.00; HPLC retention time=2.477 min (gradient of solvent B-0 to 100%; wavelength 220 nM).

N-(7-tert-Butyl-7H-pyrrolo[2,3-d]pyrimidin-2-yl)-4-methyl-benzamide (10): Under a nitrogen atmosphere, pyrrolopyrimidine 8 (199 mg, 1.05 mmol) was dissolved in 15 ml of THF. To this solution was added triethylamine (0.60 ml, 4.21 mmol) and 4-methylbenzoyl chloride (9) (0.42 ml, 3.16 mmol). The mixture was stirred at room temperature. After 45 min the mixture was diluted with a saturated solution of NaHCO_{3(aq)} and methylene chloride. The layers were separated and the aqueous portion was extracted twice more with methylene chloride. The organic phases were combined, dried over MgSO₄ and then concentrated. To a stirring solution of the residue in 15 ml of methanol was added 3 ml of a 2 N solution of NaOH_{(aq)}. After stirring for 1.5 h the mixture was diluted with a saturated solution of NaHCO_{3(aq)} and EtOAc. The layers were separated and the aqueous portion was extracted once more with EtOAc. The organic layers were combined, dried over MgSO₄ and then concentrated. The crude product was purified by silica gel column chromatography (EtOAc:hexanes, 1:4) to give the product 10 as a beige solid (222 mg, 69%): m/z calcd. for C18H20N4O: 308.39 found: 309.05; HPLC retention time=3.686 min (gradient of solvent B-0 to 100%; wavelength 220 nM).

N-(7-tert-Butyl-5-iodo-7H-pyrrolo[2,3-d]pyrimidin-2-yl)-4-methyl-benzamide (11): To a solution of the amide 10 (222 mg, 0.72 mmol) in THF was added NIS (202 mg, 1.25 mmol). The reaction flask was covered to block exposure to light and the mixture was stirred at room temperature. After 17.5 h the solvent was removed in vacuo and the residue was diluted with a saturated solution of NaHCO_{3(aq)} and methylene chloride. The layers were separated and the aqueous portion was extracted three times more with methylene chloride. The organic phases were combined, dried over MgSO₄ and then concentrated. The crude product was purified by silica gel column chromatography (EtOAc:hexanes, 1:5) to yield the iodinated product 11 (185 mg, 59%) as a brown solid: m/z calcd. for C18H19IN4O: 434.28 found: 435.00; HPLC retention time=4.031 min (gradient of solvent B-0 to 100%; wavelength 220 nM).

### 6.21. Preparation of 7-tert-Butyl-2-(4-methyl-benzoylamino)-7H-pyrrolo[2,3-d]pyrimidine-5-carboxylic acid ethylamide

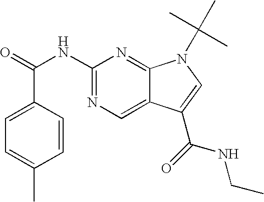

Under a blanket of nitrogen and in a scintillating vial, amide 11 (35 mg, 0.081 mmol) was dissolved in 1 ml of DMF. The solution was degassed using nitrogen and then trans-dichlorobis(triphenylphosphine)palladium (5.6 mg, 0.0081 mmol) was added. After degassing with nitrogen once more the mixture was bubbled through with carbon monoxide for 3 min. A 2 M solution of ethylamine in THF (0.081 ml, 0.162 mmol) was added to the mixture and the vial was sealed. The mixture was stirred at 80° C. After stirring for 12 h the mixture was diluted with EtOAc and filtered through Celite. The filtrate was concentrated and the residue was purified by prep-HPLC to yield the title compound (19 mg, 61%) as a white solid: ¹H NMR (300 MHz, MeOD) δ 9.34 (s, 1H), 8.49 (s, 1H), 7.99 (d, J=9.0 Hz, 2H), 7.41 (d, J=8.0 Hz, 2H), 3.44 (q, J=7.5 Hz, 2H), 2.46 (s, 3H), 1.87 (s, 9H), and 1.26 (t, J=7.2 Hz, 3H); m/z calcd. for C21H25N5O2: 379.47 found: 380.25; HPLC retention time=3.620 min (gradient of solvent B-0 to 100%; wavelength 220 nM).

### 6.22. Preparation of 7-tert-Butyl-2-(4-methyl-benzoylamino)-7H-pyrrolo[2,3-d]pyrimidine-5-carboxylic acid (pyridin-3-ylmethyl)-amide

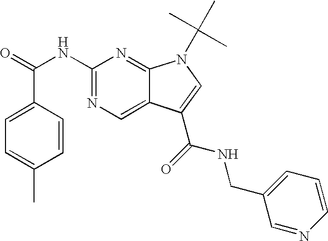

Under a blanket of nitrogen and in a scintillating vial, amide 11 (35 mg, 0.081 mmol) was dissolved in 1 ml of DMF. The solution was degassed using nitrogen and then trans-dichlorobis(triphenylphosphine)palladium (5.6 mg, 0.0081 mmol) was added. After degassing with nitrogen once more the mixture was bubbled through with carbon monoxide for 3 min. To the solution was added 3-(aminomethyl)pyridine (0.017 ml, 0.162 mmol) and the vial was sealed. The mixture was stirred at 80° C. After stirring for 12 h the mixture was diluted with EtOAc and filtered through Celite. The filtrate was concentrated and the residue was purified by prep-HPLC to yield the title compound (25 mg, 70%) as a white solid: ¹H NMR (300 MHz, MeOD) δ 9.34 (s, 1H), 8.79 (s, 1H) 8.67 (d, J=5.1 Hz, 1H), 8.49 (s, 1H), 8.37 (d, J=8.0 Hz, 1H), 7.97 (d, J=8.6 Hz, 2H), 7.84 (dd, J=5.9, 2.4 Hz, 1H), 7.40 (d, J=8.3 Hz, 2H), 4.73 (s, 2H), 2.46 (s, 3H), and 1.87 (s, 9H); m/z calcd. for C25H26N6O2: 442.52 found: 443.40; HPLC retention time=3.196 min (gradient of solvent B-0 to 100%; wavelength 220 nM).

### 6.23. Preparation of 7-tert-Butyl-2-(4-methyl-benzoylamino)-7H-pyrrolo[2,3-d]pyrimidine-5-carboxylic acid (pyridin-2-ylmethyl)-amide

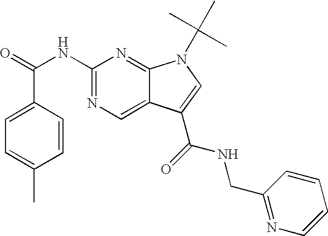

Under a blanket of nitrogen and in a scintillating vial, amide 11 (35 mg, 0.081 mmol) was dissolved in 1 ml of DMF. The solution was degassed using nitrogen and then trans-dichlorobis(triphenylphosphine)palladium (5.6 mg, 0.0081 mmol) was added. After degassing with nitrogen once more the mixture was bubbled through with carbon monoxide for 3 min. To the solution was added 2-(aminomethyl)pyridine (0.017 ml, 0.162 mmol) and the vial was sealed. The mixture was stirred at 80° C. After stirring for 12 h the mixture was diluted with EtOAc and filtered through Celite. The filtrate was concentrated and the residue was purified by prep-HPLC to yield the title compound (19 mg, 52%) as a beige solid: ¹H NMR (300 MHz, MeOD) δ 9.34 (s, 1H), 8.65 (d, J=4.5 Hz, 1H), 8.57 (s, 1H), 8.19 (td, J=7.8, 1.5 Hz, 1H), 7.98 (d, J=8.1 Hz, 2H), 7.78 (d, J=7.8 Hz, 1H), 7.64 (app t, J=6.3 Hz, 1H), 7.41 (d, J=7.8 Hz, 2H), 4.81 (s, 2H), 2.46 (s, 3H), and 1.89 (s, 9H); m/z calcd. for C25H26N6O2: 442.52 found: 443.35; HPLC retention time=3.211 min (gradient of solvent B-0 to 100%; wavelength 220 nM).

### 6.24. Preparation of 7-tert-Butyl-2-(4-methyl-benzoylamino)-7H-pyrrolo[2,3-d]pyrimidine-5-carboxylic acid (2-dimethylamino-ethyl)-amide

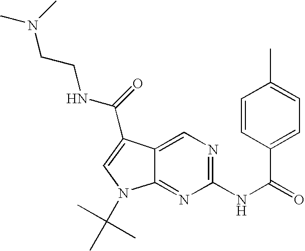

Under a blanket of nitrogen and in a scintillating vial, amide 11 (35 mg, 0.081 mmol) was dissolved in 1 ml of DMF. The solution was degassed using nitrogen and then trans-dichlorobis(triphenylphosphine)palladium (5.6 mg, 0.0081 mmol) was added. After degassing with nitrogen once more the mixture was bubbled through with carbon monoxide for 3 min. N,N-dimethyl ethylene diamine (0.014 ml, 0.162 mmol) was added to the mixture and the vial was sealed. The mixture was stirred at 80° C. After stirring for 12 h the mixture was diluted with EtOAc and filtered through Celite. The filtrate was concentrated and the residue was purified by prep-HPLC to yield the title compound (8.9 mg, 26%) as a beige solid: ¹H NMR (300 MHz, MeOD) δ 9.34 (s, 1H), 8.36 (s, 1H), 7.95 (d, J=8.3 Hz, 2H), 7.39 (d, J=8.0 Hz, 2H), 3.77 (t, J=5.7 Hz, 2H), 3.40 (t, J=5.8 Hz, 2H), 3.02 (s, 6H) 2.46 (s, 3H), and 1.86 (s, 9H); m/z calcd. for C23H30N6O2: 422.53 found: 423.30; HPLC retention time=3.138 min (gradient of solvent B-0 to 100%; wavelength 220 nM).

### 6.25. Preparation of 7-tert-Butyl-2-(4-methyl-benzoylamino)-7H-pyrrolo[2,3-d]pyrimidine-5-carboxylic acid methylamide

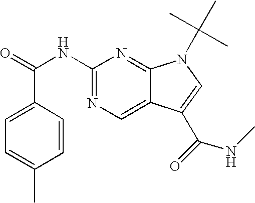

Under a blanket of nitrogen and in a scintillating vial, amide 11 (35 mg, 0.081 mmol) was dissolved in 1 ml of DMF. The solution was degassed using nitrogen and then trans-dichlorobis(triphenylphosphine)palladium (5.6 mg, 0.0081 mmol) was added. After degassing with nitrogen once more the mixture was bubbled through with carbon monoxide for 3 min. A 2 M solution of methylamine in THF (0.08 ml, 0.162 mmol) was added to the mixture and the vial was sealed. The mixture was stirred at 80° C. After stirring for 12 h the mixture was diluted with EtOAc and filtered through Celite. The filtrate was concentrated and the residue was purified by prep-HPLC to yield the title compound (23 mg, 77%) as a white solid: ¹H NMR (300 MHz, MeOD) δ 9.35 (s, 1H), 8.48 (s, 1H), 8.00 (d, J=8.6 Hz, 2H), 7.42 (d, J=7.2 Hz, 2H), 2.94 (s, 3H), 2.47 (s, 3H), and 1.88 (s, 9H); m/z calcd. for C20H23N5O2: 365.44 found: 366.25; HPLC retention time=3.443 min (gradient of solvent B-0 to 100%; wavelength 220 nM).

### 6.26. Preparation of 6-Amino-1-tert-butyl-1H-pyrrolo[2,3-b]pyridine-3-carbonitrile

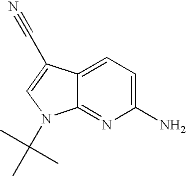

5-Amino-1-tert-butyl-1H-pyrrole-3-carbonitrile (15): To the sodium derivative of formyl-succinonitrile (14) (A. Brodrick and D. G. Wibberley, J.C.S. Perkin I, 1975, 1911) (1.0 g, 7.7 mmol) dissolved in ethanol was added 2 ml of acetic acid and then tert-butyl amine (0.85 ml, 8.1 mmol). The solution was stirred at reflux. After 45 min the mixture was cooled to room temperature. To the stirring solution was added a solution of KOH (2.68 g, 47.7 mmol) in ethanol. The resulting mixture was stirred again at reflux. After 45 min the reaction was cooled to room temperature and the solvent was removed with the rotary evaporator. The residue was diluted with water and EtOAc. The layers were partitioned and the aqueous layer was extracted twice more with EtOAc. The organic phases were combined, dried over MgSO₄ and concentrated to yield the pyrrole 15 (791 mg, 63%): ¹H NMR (300 MHz, (MeOD) δ 7.11 (d, J=2.3 Hz, 1H), 5.67 (d, J=2.2 Hz, 1H), 1.61 (s, 9H); m/z calcd. for C9H13N3: 163.22 found: 163.95; HPLC retention time=1.550 min (Column: Luna C8 4.6×50 mm, Gradient time: 3 min, flow rate: 2 ml/min, gradient of solvent B-0 to 100%; wavelength 220 nM).

6-Amino-1-tert-butyl-1H-pyrrolo[2,3-b]pyridine-3-carbonitrile (17): To a solution of pyrrole 4 (500 mg, 3.05 mmol) in 50 ml of EtOH was added 3,3-dimethoxypropionitrile (16) (350 mg, 3.05 mmol) and then 1 ml of concentrated hydrochloric acid. The solution was stirred at reflux. After 2 h the solvent was removed with the rotary evaporator. The residue was diluted with water and then neutralized with 1 N NaOH_{(aq)}. The aqueous mixture was extracted with EtOAc. The organic layer was separated, dried over MgSO₄ and concentrated. The crude product was purified by silica gel column chromatography to yield the pyrrolopyridine 17 (607 mg, 93%): ¹H NMR (400 MHz, (CDCl₃) δ 7.93 (d, J=8.8 Hz, 1H), 7.57 (s, 1H), 6.60 (d, J=8.8 Hz, 1H), 1.76 (s, 9H); m/z calcd. for C12H14N4: 214.27 found: 214.90; HPLC retention time=3.395 min (Column: ShimPack VP-ODS 50×4.6, Gradient time: 4 min, flow rate: 2.5 ml/min, gradient of solvent B-0 to 100%; wavelength 220 nM).

### 6.27. Preparation of 1-tert-Butyl-6-(4-methyl-benzoylamino)-1H-pyrrolo[2,3-b]pyridine-3-carboxylic acid

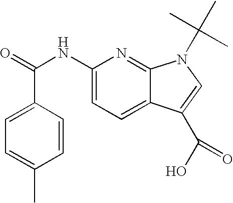

6-Amino-1-tert-butyl-1H-pyrrolo[2,3-b]pyridine-3-carboxylic acid ethyl ester (18): To a solution of the pyrrolopyridine 17 (150 mg, 0.70 mmol) in 20 ml of EtOH was added 5 ml of sulfuric acid. The solution was stirred at reflux overnight. The solvent was then removed in vacuo. The residue was diluted with water and then neutralized with 1 N NaOH_{(aq)}. The aqueous mixture was extracted with EtOAc. The organic layer was separated, dried over MgSO₄ and concentrated. The crude product was purified by prep-HPLC to yield the ester 18: ¹H NMR (400 MHz, (CDCl₃) δ 9.35 (s, 2H), 8.44 (d, J=8.8 Hz, 1H), 7.71 (s, 1H), 6.72 (d, J=8.8 Hz, 1H), 4.36 (q, J=7.2 Hz, 2H), 1.76 (s, 9H), 1.39 (t, J=7.2 Hz, 3H); m/z calcd. for C14H19N3O2: 261.33 found: 261.95; HPLC retention time=3.625 min (Column: ShimPack VP-ODS 50×4.6, Gradient time: 4 min, flow rate: 2.5 ml/min, gradient of solvent B-0 to 100%; wavelength 220 nM).

1-tert-Butyl-6-(4-methyl-benzoylamino)-1H-pyrrolo[2,3-b]pyridine-3-carboxylic acid ethyl ester (20): To a solution of ester 7 (1.2 g, 5.6 mmol) in pyridine was added p-toluoyl chloride (19) (1.02 ml, 11.2 mmol). The reaction was stirred at room temperature. After stirring for 2 h the solvent was removed with the rotary evaporator. The residue was diluted with EtOAc and then washed with brine. The organic layer was dried over MgSO₄ and concentrated. The crude product was purified by prep-HPLC to yield the amide 20 (1.37 g, 65%): ¹H NMR (400 MHz, (CDCl₃) δ 8.45 (d, J=8.8 Hz, 1H), 8.22 (d, J=8.8 Hz, 1H), 8.00 (s, 1H), 7.86 (d, J=8.0 Hz, 2H), 7.28 (d, J=8.0 Hz, 2H), 4.36 (q, J=7.2 Hz, 2H), 2.40 (s, 3H), 1.80 (s, 9H), 1.41 (t, J=7.6 Hz, 3H); m/z calcd. for C22H25N3O3: 379.46 found: 379.95; HPLC retention time=4.590 min (Column: ShimPack VP-ODS 50×4.6, Gradient time: 4 min, flow rate: 3.0 ml/min, gradient of solvent B-0 to 100%; wavelength 220 nM).

1-tert-Butyl-6-(4-methyl-benzoylamino)-1H-pyrrolo[2,3-b]pyridine-3-carboxylic acid (21): To a solution of the ester 20 (83 mg, 0.218 mmol) in ethanol was added 4 ml of 1 N NaOH_{(aq)}. The mixture was stirred at 70° C. overnight. The mixture was then diluted with EtOAc and the layers were separated. The aqueous layer was acidified with 1 N HCl_{(aq)}. The precipitate was filtered to give the desired acid 21: m/z calcd. for C20H21N3O3: 351.41 found: 351.95.

### 6.28. Preparation of 1-tert-Butyl-6-(4-methyl-benzoylamino)-1H-pyrrolo[2,3-b]pyridine-3-carboxylic acid isopropylamide

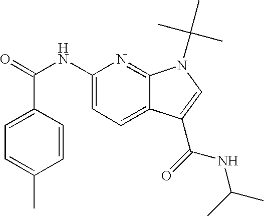

To a solution of the acid 21 (30 mg, 0.08 mmol) in DMF was added isopropylamine (0.015 ml, 0.17 mmol), then N,N,N′,N′-Tetramethyl-O-(7-azabenzotriazol-1-yl)uronium hexafluorophosphate (65 mg, 0.17 mmol) and then triethylamine (0.023 ml, 0.17 mmol). The solution was stirred at room temperature. After 12 h the mixture was concentrated. The residue was purified by prep-HPLC to yield the title compound (3.4 mg, 11%): 1H NMR (400 MHz, (CDCl3) δ 8.53 (bs, !H), 8.29 (d, J=8.8 Hz, 1H), 8.17 (d, J=8.8 Hz, 1H), 7.94 (s, 1H), 7.86 (d, J=8.0 Hz, 2H), 7.33 (d, J=8.0 Hz, 2H), 5.88 (bs, 1H), 4.39-4.32 (m, 1H), 2.45 (s, 3H), 1.79 (s, 9H), 1.32 (d, J=6.8 Hz, 6H); m/z calcd. for C23H28N4O2: 392.51 found: 393.00; HPLC retention time=4.193 min (Column: ShimPack VP-ODS 50×4.6, Gradient time: 4 min, flow rate: 3.0 ml/min, gradient of solvent B-15 to 100%; wavelength 220 nM).

### 6.29. Preparation of 1-tert-Butyl-6-(4-methyl-benzoylamino)-1H-pyrrolo[2,3-b]pyridine-3-carboxylic acid ethylamide

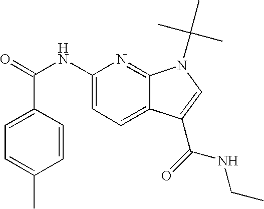

To a solution of the acid 21 (50 mg, 0.14 mmol) in DMF was added ethylamine (0.140 ml, 0.28 mmol), then N,N,N′,N′-Tetramethyl-O-(7-azabenzotriazol-1-yl)uronium hexafluorophosphate (108 mg, 0.28 mmol) and then triethylamine (0.041 ml, 0.28 mmol). The solution was stirred at room temperature. After 12 h the mixture is concentrated. The residue is purified by prep-HPLC to yield the title compound (17 mg, 32%): ¹H NMR (400 MHz, (CDCl₃) δ 8.72 (bs, 1H), 8.21 (dd, J=8.8, 6.8 Hz, 2H), 7.94 (s, 1H), 7.85 (d, J=8.0 Hz, 2H), 7.30 (d, J=8.4 Hz, 2H), 6.36 (bs, 1H), 3.51 (q, J=7.2 Hz, 2H), 2.43 (s, 3H), 1.76 (s, 9H), 1.26 (t, J=7.2 Hz, 3H); m/z calcd. for C22H26N4O2: 378.48 found: 379.00; HPLC retention time=5.168 min (Column: ShimPack VP-ODS 50×4.6, Gradient time: 5 min, flow rate: 3.0 ml/min, gradient of solvent B-10 to 100%; wavelength 220 nM).

### 6.30. Preparation of 1-tert-Butyl-6-(4-methyl-benzoylamino)-1H-pyrrolo[2,3-b]pyridine-3-carboxylic acid isobutyl-amide

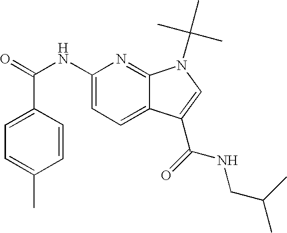

To a solution of the acid 21 (50 mg, 0.14 mmol) in DMF was added isobutylamine (0.028 ml, 0.28 mmol), then N,N,N′,N′-Tetramethyl-O-(7-azabenzotriazol-1-yl)uronium hexafluorophosphate (108 mg, 0.28 mmol) and then triethylamine (0.041 ml, 0.28 mmol). The solution was stirred at room temperature. After 12 h the mixture is concentrated. The residue is purified by prep-HPLC to yield the title compound (22 mg, 24%): ¹H NMR (400 MHz, (CDCl₃) δ 8.60 (bs, 1H), 8.27 (d, J=8.8 Hz, 1H), 8.17 (d, J=8.4 Hz, 1H), 7.97 (s, 1H), 7.86 (d, J=8.4 Hz, 2H), 7.33 (d, J=7.6 Hz, 2H), 6.20 (bs, 1H), 3.34 (d, J=6.8 Hz, 2H), 2.45 (s, 3H), 1.99-1.90 (m, 1H), 1.76 (s, 9H), 1.01 (d, J=6.8 Hz, 3H); m/z calcd. for C24H30N4O2: 406.53 found: 407.05; HPLC retention time=4.796 min (Column: ShimPack VP-ODS 50×4.6, Gradient time: 5 min, flow rate: 3.0 ml/min, gradient of solvent B-30 to 100%; wavelength 220 nM).

### 6.31. Preparation of 1-tert-Butyl-6-(4-methyl-benzoylamino)-1H-pyrrolo[2,3-b]pyridine-3-carboxylic acid dimethylamide

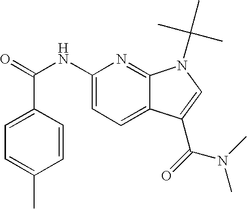

To a solution of the acid 21 (50 mg, 0.14 mmol) in DMF was added dimethylamineamine (0.140 ml, 0.28 mmol), then N,N,N′,N′-Tetramethyl-O-(7-azabenzotriazol-1-yl)uronium hexafluorophosphate (108 mg, 0.28 mmol) and then triethylamine (0.041 ml, 0.28 mmol). The solution was stirred at room temperature. After 12 h the mixture is concentrated. The residue is purified by prep-HPLC to yield the title compound (5.3 mg, 10%): ¹H NMR (400 MHz, (CDCl₃) δ 8.42 (bs, 1H), 8.26 (d, J=8.8 Hz, 1H), 8.06 (d, J=9.6 Hz, 1H), 7.86 (d, J=7.2 Hz, 2H), 7.64 (s, 1H), 7.32 (d, J=7.6 Hz, 2H), 3.17 (s, 6H), 2.44 (s, 3H), 1.79 (s, 9H); m/z calcd. for C22H26N4O2: 378.48 found: 379.00; HPLC retention time=3.455 min (Column: ShimPack VP-ODS 50×4.6, Gradient time: 4 min, flow rate: 3.0 ml/min, gradient of solvent B-40 to 100%; wavelength 220 nM).

### 6.32. Preparation of 1-tert-Butyl-6-(4-methyl-benzoylamino)-1H-pyrrolo[2,3-b]pyridine-3-carboxylic acid diethylamide

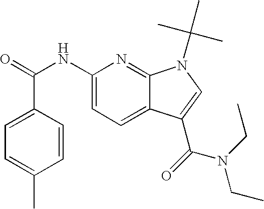

To a solution of the acid 21 (50 mg, 0.14 mmol) in DMF was added diethylamine (0.05 ml, 0.28 mmol), then N,N,N′,N′-tetramethyl-O-(7-azabenzotriazol-1-yl)uronium hexafluorophosphate (108 mg, 0.28 mmol) and then triethylamine (0.041 ml, 0.28 mmol). The solution was stirred at room temperature. After 12 hours the mixture is concentrated. The residue is purified by prep-HPLC to yield the title compound (9.9 mg, 17%): ¹H NMR (400 MHz, (CDCl₃) δ 8.49 (bs, 1H), 8.24 (d, J=8.8 Hz, 1H), 8.00 (d, J=8.8 Hz, 1H), 7.86 (d, J=8.0 Hz, 2H), 7.65 (s, 1H), 7.33 (d, J=8.4 Hz, 2H), 3.61 (q, J=7.2 Hz, 4H), 2.45 (s, 3H), 1.79 (s, 9H), 1.26 (t, J=7.2 Hz, 6H); m/z calcd. for C24H30N4O2: 406.53 found: 407.00; HPLC retention time=4.545 min (Column: ShimPack VP-ODS 50×4.6, Gradient time: 5 min, flow rate: 3.0 ml/min, gradient of solvent B-30 to 100%; wavelength 220 nM).

### 6.33. Preparation of 1-tert-Butyl-6-fluoro-1H-pyrazolo[3,4-b]pyridine-3-carboxylic acid

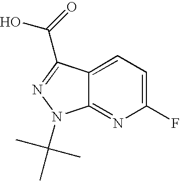

(2,6-Difluoro-pyridin-3-yl)-oxo-acetic acid tert-butyl ester (23): To a solution of 2,6-difluoropyridine (22) (2.7 ml, 30 mmol) in 30 ml of THF at −78° C. was added dropwise a freshly prepared solution of lithium diisopropylamine (32 mmol). The resulting solution was maintained at −78° C. for 30 min. To the stirring solution was added dropwise a preloaded solution of di-tert-butyl oxylate (7.7 g, 38 mmol) in 30 ml of THF at −78° C. The reaction mixture was stirred at −78° C. for 30 min and then at −20° C. for 20 min. The solution was quenched with a saturated solution of NH₄Cl_{(aq)} and then diluted with Et₂O. The layers were separated and the organic layer was dried over Na₂SO₄ and then concentrated in vacuo to yield the product 23 (6.93 g, 95%) as a yellow oil: ¹H NMR (300 MHz, (CDCl₃) δ 8.49 (dd, 1H), 7.04 (dd, J_{HH}=8.2 Hz, J_{HF}=2.9 Hz, 1H), 1.61 (s, 9H).

(tert-Butyl-hydrazono)-(2,6-difluoro-pyridin-3-yl)-acetic acid tert-butyl ester (24): To a solution of the difluoropyridine 23 (8.0 g, 32.9 mmol) in EtOH was added tert-butylhydrazine (4.1 g, 32.9 mmol) and triethylamine (4.58 ml, 32.9 mmol). The reaction was stirred at 60° C. After stirring for 2 h the mixture was concentrated in vacuo. The residue was diluted with brine and methylene chloride. The layers were separated and the organic layer was dried over MgSO₄ and concentrated. The crude product was purified by silica gel column chromatography to yield the product 24 (2.25 g, 22%): ¹H NMR (400 MHz, (CDCl₃) δ 7.82 (dd, 1H), 7.04 (dd, J_{HH}=8.0 Hz, J_{HF}=3.0 Hz, 1H), 1.47 (s, 9H), 1.27 (s, 9H).

1-tert-Butyl-6-fluoro-1H-pyrazolo[3,4-b]pyridine-3-carboxylic acid tert-butyl ester (25): To a solution of 24 (2.3 g, 7.35 mmol) in THF was added sodium hydride (340 mg, 8.81 mmol). The reaction was stirred at 70° C. and followed using TLC. Upon completion the mixture was quenched with a saturated solution of NH₄Cl_{(aq)} and then diluted with brine. The layers were separated and the organic layer was dried over MgSO₄ and concentrated in vacuo. The crude product was purified by silica gel column chromatography to yield the ester 25 (1.2 g, 56%): ¹H NMR (300 MHz, (CDCl₃) δ 8.45 (dd, 1H), 6.90 (dd, J_{HH}=8.6 Hz, J_{HF}=1.4 Hz, 1H), 1.86 (s, 9H), 1.70 (s, 9H); m/z calcd. for C15H20FN3O2: 293.34 found: 293.90; HPLC retention time=3.726 min (Gradient time: 3 min, flow rate: 2.5 ml/min, gradient of solvent B-50 to 100%; wavelength 220 nM).

1-tert-Butyl-6-fluoro-1H-pyrazolo[3,4-b]pyridine-3-carboxylic acid (26): To a solution of the ester 25 (1.2 g, 4.1 mmol) in 40 ml of methylene chloride was added 5 ml of trifluoroacetic acid. After stirring for 4 h the mixture was concentrated to yield the acid 26.

### 6.34. Preparation of 1-tert-Butyl-6-(4-methyl-benzoylamino)-1H-pyrazolo[3,4-b]pyridine-3-carboxylic acid (1-ethyl-propyl)-amide

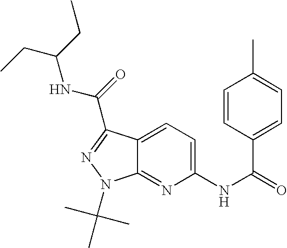

6-Amino-1-tert-butyl-1H-pyrazolo[3,4-b]pyridine-3-carboxylic acid (1-ethyl-propyl)-amide (27): To a solution of acid 26 (158 mg, 0.667 mmol), triethylamine (0.1 ml, 0.733 mmol), EDCl (140 mg, 0.733 mmol) and HOAt (100 mg, 0.733 mmol) in methylene chloride was added 1-ethylpropane (58 mg, 0.667 mmol). The mixture was stirred at room temperature overnight. The reaction was then washed with brine. The organic layer was separated, dried over MgSO₄ and concentrated to give a yellow oil. The crude intermediate was taken up in 10 ml of 7 N ammonia dissolved in methanol. The solution was stirred at 140° C. After 36 h the mixture was concentrated. The crude product was purified by prep-HPLC to yield the amide 27 (60 mg, 30%) as a clear oil: m/z calcd. for C16H25N5O: 303.41 found: 304.20

1-tert-Butyl-6-(4-methyl-benzoylamino)-1H-pyrazolo[3,4-b]pyridine-3-carboxylic acid (1-ethyl-propyl)-amide (29): To a solution of amide 7 (80 mg, 0.264 mmol) in 3 ml of pyridine was added p-toluoyl chloride (0.087 ml, 0.66 mmol). The reaction was stirred at room temperature and followed using TLC. After 4 h of stirring the solvent was removed with the rotary evaporator. The residue was diluted with methylene chloride and then washed with a saturated solution of NaHCO_{3(aq)} and brine, The organic layer was dried over MgSO₄ and concentrated. The crude product was purified by prep-HPLC to yield the title compound (57 mg, 51%) as a white solid: ¹H NMR (300 MHz, (CDCl₃) δ 8.71 (d, J=9.0 Hz, 1H), 8.52 (bs, 1H), 8.39 (d, J=8.7 Hz, 1H), 7.89 (d, J=8.4 Hz, 2H), 7.36 (d, J=7.8 Hz, 2H), 6.75 (d, J=9.5 Hz, 1H), 4.11-3.97 (m, 1H), 2.47 (s, 3H), 1.85 (s, 9H), 1.71-167 (m, 2H), 1.61-1.55 (m, 2H), 1.02 (t, J=7.2 Hz, 6H); m/z calcd. for C24H31N5O2: 421.55 found: 422.30; HPLC retention time=4.731 min (Gradient time: 3 min, flow rate: 3 ml/min, gradient of solvent B-40 to 100%; wavelength 220 nM).

### 6.35. Preparation of 1-tert-Butyl-6-(4-methyl-benzoylamino)-1H-pyrazolo[3,4-b]pyridine-3-carboxylic acid isopropylamide

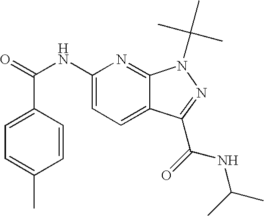

6-Amino-1-tert-butyl-1H-pyrazolo[3,4-b]pyridine-3-carboxylic acid isopropylamide (29): To a solution of acid 26 (200 mg, 0.844 mmol), triethylamine (0.142 ml, 1.02 mmol), EDCl (198 mg, 1.02 mmol) and HOAt (137 mg, 1.02 mmol) in 5 ml of methylene chloride was added isopropylamine (0.072 ml, 0.844 mmol). The mixture was stirred at room temperature overnight. The reaction was then washed with a saturated solution of NaHCO_{3(aq)} and brine. The organic layer was separated, dried over MgSO₄ and concentrated to give a yellow solid. The crude intermediate was taken up in 7 N ammonia dissolved in methanol. The solution was stirred at 140° C. After 24 h the mixture was concentrated. The crude product was purified by prep-HPLC to yield the amide 29 (99 mg, 43%) as a white solid: m/z calcd. for C14H21N5O: 275.36 found: 276.1.

1-tert-Butyl-6-(4-methyl-benzoylamino)-1H-pyrazolo[3,4-b]pyridine-3-carboxylic acid isopropylamide (30): To a solution of amide 29 (60 mg, 0.218 mmol) in 4 ml of pyridine was added p-toluoyl chloride (0.051 ml, 0.436 mmol). The reaction was stirred at room temperature. After 4 h of stirring the solvent was removed with the rotary evaporator. The residue was diluted with 50 ml of methylene chloride and then washed with a saturated solution of NaHCO_{3(aq)} and brine. The organic layer was dried over MgSO₄ and concentrated. The crude product was purified by prep-HPLC to yield the title compound (38 mg, 44%) as a white solid: ¹H NMR (300 MHz, (CDCl₃) δ 8.70 (d, J=8.7 Hz, 1H), 8.51 (bs, 1H), 8.38 (d, J=8.7 Hz, 1H), 7.89 (d, J=8.4 Hz, 2H), 7.36 (d, J=7.8 Hz, 2H), 6.84 (d, J=9.0 Hz, 1H), 4.42-4.29 (m, 1H), 2.48 (s, 3H), 1.85 (s, 9H), 1.34 (d, J=6.6, 6H); m/z calcd. for C22H27N5O2: 393.49 found: 394.30; HPLC retention time=4.371 min (Gradient time: 3 min, flow rate: 3 ml/min, gradient of solvent B-50 to 100%; wavelength 220 nM).

### 6.36. Preparation of 6-Amino-1-tert-butyl-1H-pyrazolo[3,4-b]pyridine-3-carboxylic acid cyclopropylamide

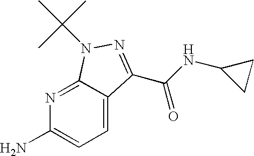

To a solution of acid 26 (150 mg, 0.632 mol), triethylamine (0.07 ml, 0.76 mmol), EDCl (145 mg, 0.76 mmol) and HOAt (103 mg, 0.76 mmol) in methylene chloride was added cyclopropylamine (36 mg, 0.632 mmol). The mixture was stirred at room temperature overnight. The reaction was then washed with a saturated solution of NaHCO_{3(aq)} and brine. The organic layer was separated, dried over MgSO₄ and concentrated. The crude intermediate was taken up in 7 N ammonia dissolved in methanol. The solution was stirred at 140° C. After 24 h the mixture was concentrated. The crude product was purified by prep-HPLC to yield the title amide (50 mg, 27%) as a white solid: m/z calcd. for C14H19N5O: 273.34 found: 274.2

1-tert-Butyl-6-(4-methyl-benzoylamino)-1H-pyrazolo[3,4-b]pyridine-3-carboxylic acid cyclopropylamide: To a solution of 6-Amino-1-tert-butyl-1H-pyrazolo[3,4-b]pyridine-3-carboxylic acid cyclopropylamide (50 mg, 0.169 mmol) in 2 ml of pyridine was added p-toluoyl chloride (0.05 ml, 0.378 mmol). The reaction was stirred at room temperature overnight. The solvent was removed with the rotary evaporator. The residue was diluted with 50 ml of methylene chloride and then washed with a saturated solution of NaHCO_{3(aq)} and brine. The organic layer was dried over MgSO₄ and concentrated. The crude product was purified by prep-HPLC to yield the title compound (25 mg, 38%) as a white solid: ¹H NMR (300 MHz, (CDCl₃) δ 8.70 (d, J=9.0 Hz, 1H), 8.51 (bs, 1H), 8.40 (d, J=9.0 Hz, 1H), 7.89 (d, J=8.1 Hz, 2H), 7.36 (d, J=8.1 Hz, 2H), 7.11 (bs, 1H), 2.97-2.87 (m, 1H), 2.47 (s, 3H), 1.83 (s, 9H), 0.95-0.88 (m, 2H), 0.75-0.70 (m, 2H); m/z calcd. for C22H25N5O2: 391.48 found: 392.45; HPLC retention time=4.201 min (Gradient time: 3 min, flow rate: 3 ml/min, gradient of solvent B-50 to 100%; wavelength 220 nM).

### 6.37. Preparation of 1-tert-Butyl-6-(3-methyl-benzoylamino)-1H-pyrazolo[3,4-b]pyridine-3-carboxylic acid isopropylamide

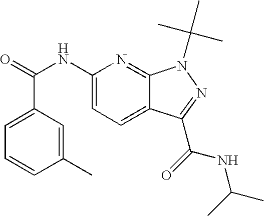

To a solution of m-toluoyl chloride (0.025 ml, 0.18 mmol) in 0.5 ml of pyridine was added a solution of the amide 29 (38 mg, 0.18 mmol) in 1.5 ml of pyridine. The resulting solution was stirred at room temperature for 3 h and then concentrated. The crude product was purified by prep-HPLC to yield the title compound as a white solid: ¹H NMR (300 MHz, (CDCl₃) δ 8.61 (d, J=8.7 Hz, 1H), 8.39 (s, 1H), 8.28 (d, J=9.0 Hz, 1H), 7.72-7.62 (m, 2H), 7.36-7.31 (m, 2H), 6.72 (d, J=7.8 Hz, 1H), 4.33-4.19 (m, 1H), 2.39 (s, 3H), 1.75 (s, 9H), 1.24 (d J=6.6, 6H); m/z calcd. for C22H27N5O2: 393.49 found: 394.35.

### 6.38. Human Proline Transporter Assay

The ability of compounds to inhibit the proline transporter was determined as follows. A human SLC6A7 cDNA was cloned into a pcDNA3.1 vector and transfected into COS-1 cells. A cell clone stably expressing proline transporter was selected for the assay.

Transfected cells were seeded at 15,000 cells per well in a 384 well plate and grown overnight. The cells were then washed with Krebs-Ringer's-HEPES-Tris (KRHT) buffer, pH 7.4, containing 120 mM NaCl, 4.7 mM KCl, 2.2 mM CaCl, 1.2 mM MgSO₄, 1.2 mM KH₂PO₄, 10 mM HEPES and 5 mM Tris. The cells were then incubated with 50 μl of KRHT buffer containing 45 nM ³H-Proline for 20 minutes at room temperature. Radiolabeled proline uptake was terminated by removing the radiolabeled proline and washing the cells rapidly three times with 100 μl of ice-cold KRHT buffer. Scintillation fluid (50 μl) was added per well, and the amount of tritiated proline present was determined using a Packard TopCount Scintillation counter.

Nonspecific uptake was determined by measuring of ³H-proline uptake in the presence of 2 mM cold proline.

The IC₅₀ of a compound was determined by measuring inhibition of four separate samples at ten concentrations, typically beginning with 10 μM followed by nine three-fold dilutions (i.e., 10, 3.3, 1.1, 0.37, 0.12, 0.41, 0.014, 0.0046, 0.0015, and 0 μM). Percent inhibitions were calculated against the control. The IC₅₀ of a compound was determined using the ten data points, each of which was an average of the four corresponding measurements.

### 6.39. Murine Proline Transporter Assay

Forebrain tissue was dissected from a wild type mouse and homogenized in 7 ml ice-cold homogenization buffer: 0.32 M sucrose, 1 mM NaHCO₃, protease inhibitor cocktail (Roche).

The brain homogenates were centrifuged at 1000×g for 10 min to remove nuclei. Supernatant was collected and re-centrifuged at 20000×g for 20 min to pellet crude synaptosomes. The synaptosomes were resuspended in ice-cold assay buffer: 122 mM NaCl, 3.1 mM KCl, 25 mM HEPES, 0.4 mM KH₂PO₄, 1.2 mM MgSO₄, 1.3 mM CaCl₂, 10 mM dextrose at pH 7.4. Resuspended synaptosomes were centrifuged again at 20000×g for 20 minutes, and pelleted synaptosomes were resuspended in assay buffer. Protein concentration was measured by DC protein assay kit (BioRad).

Proline transport assay was performed in 100 μl reaction mix consisting of 10 μg synaptosomes, 1 μCi/0.24 μM [H3]-proline in assay buffer for a time between 0 to 20 minutes at room temperature. The reaction was terminated by rapid filtration through GF/B filter plate (Millipore) followed by three rapid washes in 200 ul ice-cold assay buffer. Fifty microliters of Microscint-20 was added to each reaction and incubated for 2 hours. The [H3]-proline transport was determined by radioactivity counting.

To determine proline transport inhibition by compounds, compounds were incubated with the reaction mixture at concentrations ranging from 0 to 10 μM (11 points, beginning at 10 um; 3-fold dilutions; 4 replicates averaged to provide one point). The baseline activity, or nonspecific activity, was measured in the presence of 0.3 mM GGFL (Enkephalin, Sigma) in the reaction. The nonspecific activity was also measured in synaptosomes of SLC6A7 knockout mice. The nonspecific activities measured by the two methods were found to be identical.

### 6.40. Human Dopamine Transporter Assay

The ability of compounds to inhibit the dopamine transporter was determined as follows. A human DAT cDNA (NM_{—}001044) was cloned into a pcDNA3.1 vector and transfected into COS-1 cells. The resulting cell lines that stably express the dopamine transporter were used for further experimentation.

Transfected cells were seeded at 15,000 cells per well in a 384 well plate and grown overnight. The cells were then washed with Krebs-Ringer's-HEPES-Tris (KRHT) buffer, pH 7.4, containing 125 mM NaCl, 4.8 mM KCl, 1.3 mM CaCl₂, 1.2 mM MgSO₄ 10 mM D-glucose, 25 mM HEPES, 1 mM sodium ascorbate and 1.2 mM KH₂PO₄. The cells were then incubated with 50 μl of KRHT buffer containing 1 μM ³H-Dopamine for 10 minutes at room temperature. Radiolabeled dopamine uptake was terminated by removing the radiolabeled dopamine and washing the cells rapidly three times with 100 μl of ice-cold KRHT buffer. Scintillation fluid (50 μl) was added per well and the amount of tritiated dopamine present was determined using a Packard TopCount Scintillation counter.

Nonspecific uptake was determined by measuring of ³H-dopamine uptake in the presence of 250 μM benztropine. The IC₅₀ of a compound was determined by measuring inhibition of four separate samples at ten concentrations, typically beginning with 10 μM followed by nine three-fold dilutions (i.e., 10, 3.3, 1.1, 0.37, 0.12, 0.41, 0.014, 0.0046, 0.0015, and 0 μM). Percent inhibitions were calculated against the control. The percentage inhibitions were calculated against the control, and the average of the quadruplicates was used for IC₅₀ calculation.

### 6.41. Human Glycine Transporter Assay

The ability of compounds to inhibit the glycine transporter was determined as follows. A human glycine transporter cDNA (NM_{—}006934) was cloned into a pcDNA3.1 vector and transfected into COS-1 cells. The resulting cell lines that stably express the glycine transporter were used for further experimentation.

Transfected cells were seeded at 15,000 cells per well in a 384 well plate and grown overnight. The cells were then washed with Krebs-Ringer's-HEPES-Tris (KRHT) buffer, pH 7.4, containing 120 mM NaCl, 4.7 mM KCl, 2.2 mM CaCl₂, 1.2 mM MgSO₄, 1.2 mM KH₂PO₄, 10 mM HEPES and 5 mM Tris. The cells were then incubated with 50 μl of KRHT buffer containing 166 nM ³H-glycine for 10 minutes at room temperature. Radiolabeled glycine uptake was terminated by removing the radiolabeled glycine and washing the cells rapidly three times with 100 μl of ice-cold KRHT buffer. Scintillation fluid (50 μl) was added per well and the amount of tritiated glycine present was determined using a Packard TopCount Scintillation counter.

Nonspecific uptake was determined by measuring ³H-glycine uptake in the presence of 2 mM cold glycine. The IC₅₀ of a compound was determined by measuring inhibition of four separate samples at ten concentrations, typically beginning with 10 μM followed by nine three-fold dilutions (i.e., 10, 3.3, 1.1, 0.37, 0.12, 0.41, 0.014, 0.0046, 0.0015, and 0 μM). Percent inhibitions were calculated against the control. The percentage inhibitions were calculated against the control, and the average of the quadruplicates was used for IC₅₀ calculation.

### 6.42. Calculating IC₅₀ Values

The IC₅₀ of a compound with regard to a given target is determined by fitting the relevant data, using the Levenburg Marquardt algorithm, to the equation:

y=A+((B−A)/(1+((C/x)̂D)))

wherein A is the minimum y value; B is the maximum y value; C is the IC₅₀; and D is the slope. The calculation of the IC₅₀ is performed using XLFit4 software (ID Business Solutions Inc., Bridgewater, N.J. 08807) for Microsoft Excel (the above equation is model 205 of that software).

### 6.43. Pharmacological Effects

A compound having a PTIC₅₀ of less than 100 nM was administered to male C57B/6 albino mice subjected to a contextual fear conditioning program using a trace conditioning protocol. The compound was administered at doses ranging from 50-200 mg/kg, and was found to recapitulate phenotypes observed in SLC6A7 KO mice in a dose-dependent manner.

In the protocol, compound was administered p.o., two hours prior to training (Day 1) and again two hours prior to testing the next day (Day 2). Generally, 10-14 mice/group were tested in each study. The two hour pretreatment interval was chosen based on PK results to achieve of peak plasma and brain tissue levels.

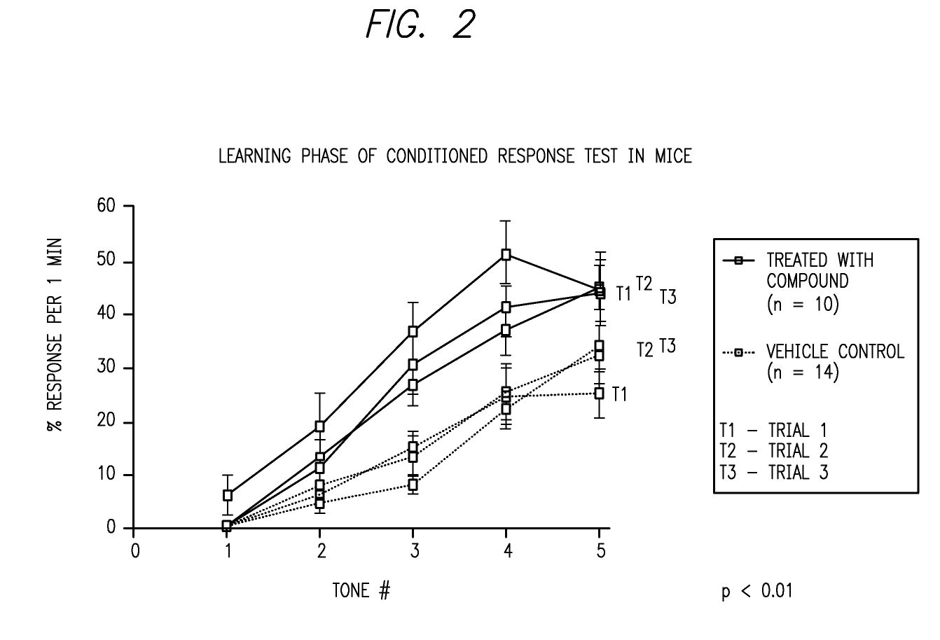

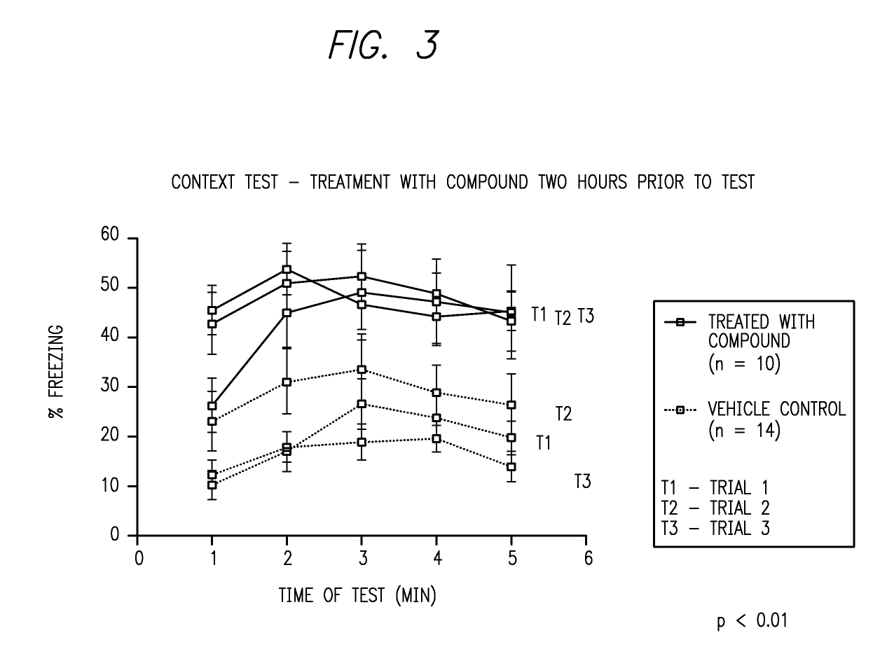

In the trace conditioning experiments, no significant effect was observed in mice dosed at 50 mg/kg, p.o., although a numerical enhancement was seen. But at doses of 100 and 200 mg/kg, p.o., significant increases in performance were observed both during training (Day 1) and testing (Day 2). As shown in FIG. 2, the compound enhanced performance during training as well as during memory testing, indicating that its effects are not changed upon repeated administration. And as shown in FIG. 3, when administered prior to the recall test but not prior to training, the compound enhanced the conditioned response.

In order to gauge whether the compound's effect changed following repeated dosing, it was administered for three days b.i.d. prior to the training day, as well as b.i.d. on the training day and prior to the test. As in the acute studies, the compound was administered two hours prior to the training session and two hours prior to the test session. Based on separate PK studies, this administration regimen was expected to provide blood levels of the compound throughout the study. Results similar to those shown in FIGS. 2 and 3 were observed, suggesting that the compound can enhance both learning and memory/recall.

The compound did not increase freezing by itself in naïve mice, as assessed in an open-field in the conditioning training apparatus, nor in mice given specific conditioning training and then placed in a novel open-field. Therefore, its effects appear to be specific to the learned response, and not due to non-specific enhancement of freezing behavior.

Each of the references (e.g., patents and patent applications) cited herein is incorporated herein in its entirety.

1-16. (canceled)

17. A method of treating schizophrenia, which comprises administering to a patient in need thereof a therapeutically effective amount of a compound of the formula: 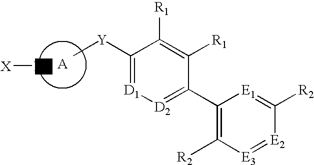
or a pharmaceutically acceptable salt thereof, wherein:
A is an optionally substituted non-aromatic heterocycle;
each of D₁ and D₂ is independently N or CR₁;
each of E₁, E₂ and E₃ is independently N or CR₂;
X is optionally substituted heteroaryl; Y is O, C(O), CH(OH), or CH₂;
each R₁ is independently hydrogen, halogen, cyano, R_{A}, OR_{A}, C(O)R_{A}, C(O)OR_{A}, C(O)N(R_{A}R_{B}), N(R_{A}R_{B}), or SO₂R_{A};
each R₂ is independently hydrogen, halogen, cyano, R_{A}, OR_{A}, C(O)R_{A}, C(O)OR_{A}, C(O)N(R_{A}R_{B}), N(R_{A}R_{B}), or SO₂R_{A};
each R_{A} is independently hydrogen or optionally substituted alkyl, aryl, arylalkyl, alkylaryl, heterocycle, heterocycle-alkyl, or alkyl-heterocycle;
and each R_{B} is independently hydrogen or optionally substituted alkyl, aryl, arylalkyl, alkylaryl, heterocycle, heterocycle-alkyl, or alkyl-heterocycle.

18. The method of claim 17, wherein the compound is of the formula: 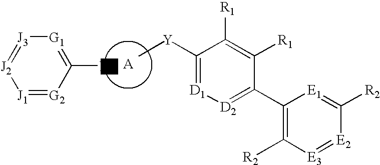
wherein:
A is an optionally substituted non-aromatic heterocycle;
each of D₁ and D₂ is independently N or CR₁;
each of E₁, E₂ and E₃ is independently N or CR₂;
each of G₁ and G₂ are independently N or CR₃;
each of J₁, J₂ and J₃ are independently N or CR₄; Y is O, C(O), CH(OH), or CH₂;
each R₁ is independently hydrogen, halogen, or (C₁₋₁₀)alkyl;
each R₂ is independently halogen, cyano, R₂A, OR₂A, or SO₂R₂A;
each R₂A is independently hydrogen or (C₁₋₁₀)alkyl optionally substituted with one or more halogens;
each R₃ is independently hydrogen, cyano, or (C₁₋₁₀)alkyl optionally substituted with one or more halogens;
and each R₄ is independently hydrogen, cyano, or (C₁₋₁₀)alkyl optionally substituted with one or more halogens.

19-23. (canceled)

24. The method of claim 17, wherein the compound is of the formula: 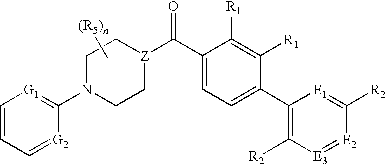
wherein:
Z is CR₅ or N;
each R₅ is independently halogen, cyano, R_{5A}, OR_{5A}, C(O)R_{5A}, C(O)OR_{5A}, C(O)N(R_{5A}R_{5B}), N(R_{5A}R_{5B}), or SO₂R_{5A};
each R_{5A} is independently hydrogen or optionally substituted alkyl, aryl, arylalkyl, alkylaryl, heterocycle, heterocycle-alkyl, or alkyl-heterocycle;
each R_{5B} is independently hydrogen or optionally substituted alkyl, aryl, arylalkyl, alkylaryl, heterocycle, heterocycle-alkyl, or alkyl-heterocycle; and
n is 0-5 if Z is CR₅, or 0-4 if Z is N.

25. The method of claim 24, wherein Z is CH, G₁ is N, and at least one R₂ is not hydrogen.

26. The method of claim 17, wherein the compound is of the formula:

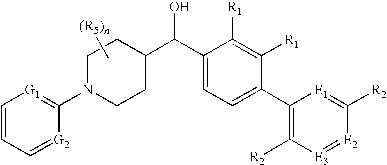

27. The method of claim 17, wherein the compound is of the formula: 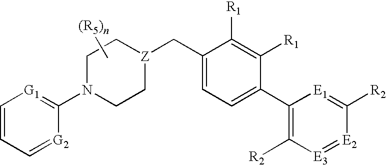
wherein:
Z is CR₅ or N;
each R₅ is independently halogen, cyano, R_{5A}, OR_{5A}, C(O)R_{5A}, C(O)OR_{5A}, C(O)N(R_{5A}R_{5B}), N(R_{5A}R_{5B}), or SO₂R_{5A};
each R_{5A} is independently hydrogen or optionally substituted alkyl, aryl, arylalkyl, alkylaryl, heterocycle, heterocycle-alkyl, or alkyl-heterocycle;
each R_{5B} is independently hydrogen or optionally substituted alkyl, aryl, arylalkyl, alkylaryl, heterocycle, heterocycle-alkyl, or alkyl-heterocycle; and
n is 0-5 if Z is CR₅, or 0-4 if Z is N.
